SEQUENCE LISTING (84 sequences)

SEQ ID NO: 1 - protein, 512 aa, Homo sapiens
MTAPWVALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 2 - protein, 115 aa, Homo sapiens
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 3 - protein, 100 aa, Artificial Sequence
  Description of Artificial Sequence: Synthetic polypeptide
GRGEAETRECIYYNANWELERTNQSGLERCEGEQDKRLHCYASWRNSSGTIELVKKGCWLDDFNCYDRQECVATEENPQVYFCCCEGNFCNERFTHLPEA

SEQ ID NO: 4 - protein, 512 aa, Artificial Sequence
  Description of Artificial Sequence: Synthetic polypeptide
MTAPWVALALLWGSLCAGSGRGEAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 5 - protein, 115 aa, Artificial Sequence
  Description of Artificial Sequence: Synthetic polypeptide
GRGEAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPT

SEQ ID NO: 6 - protein, 100 aa, Artificial Sequence
  Description of Artificial Sequence: Synthetic polypeptide
GRGEAETRECIYYNANWELERTNQSGLERCEGEQDKRLHCYASWANSSGTIELVKKGCWLDDFNCYDRQECVATEENPQVYFCCCEGNFCNERFTHLPEA

SEQ ID NO: 7 - DNA, 1536 bp, Homo sapiens
atgacggcgccctgggtggccctcgccctcctctggggatcgctgtgcgccggctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgaacgcttcactcatttgccagaggctgggggcccggaagtcacgtacgagccacccccgacagcccccaccctgctcacggtgctggcctactcactgctgcccatcgggggcctttccctcatcgtcctgctggccttttggatgtaccggcatcgcaagcccccctacggtcatgtggacatccatgaggaccctgggcctccaccaccatcccctctggtgggcctgaagccactgcagctgctggagatcaaggctcgggggcgctttggctgtgtctggaaggcccagctcatgaatgactttgtagctgtcaagatcttcccactccaggacaagcagtcgtggcagagtgaacgggagatcttcagcacacctggcatgaagcacgagaacctgctacagttcattgctgccgagaagcgaggctccaacctcgaagtagagctgtggctcatcacggccttccatgacaagggctccctcacggattacctcaaggggaacatcatcacatggaacgaactgtgtcatgtagcagagacgatgtcacgaggcctctcatacctgcatgaggatgtgccctggtgccgtggcgagggccacaagccgtctattgcccacagggactttaaaagtaagaatgtattgctgaagagcgacctcacagccgtgctggctgactttggcttggctgttcgatttgagccagggaaacctccaggggacacccacggacaggtaggcacgagacggtacatggctcctgaggtgctcgagggagccatcaacttccagagagatgccttcctgcgcattgacatgtatgccatggggttggtgctgtgggagcttgtgtctcgctgcaaggctgcagacggacccgtggatgagtacatgctgccctttgaggaagagattggccagcacccttcgttggaggagctgcaggaggtggtggtgcacaagaagatgaggcccaccattaaagatcactggttgaaacacccgggcctggcccagctttgtgtgaccatcgaggagtgctgggaccatgatgcagaggctcgcttgtccgcgggctgtgtggaggagcgggtgtccctgattcggaggtcggtcaacggcactacctcggactgtctcgtttccctggtgacctctgtcaccaatgtggacctgccccctaaagagtcaagcatc

SEQ ID NO: 8 - DNA, 345 bp, Homo sapiens
gggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgaacgcttcactcatttgccagaggctgggggcccggaagtcacgtacgagccacccccgacagcccccacc

SEQ ID NO: 9 - protein, 513 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 10 - protein, 115 aa, Homo sapiens
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 11 - protein, 100 aa, Artificial Sequence
  Description of Artificial Sequence: Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEM

SEQ ID NO: 12 - DNA, 1539 bp, Homo sapiens
atgggagctgctgcaaagttggcgtttgccgtctttcttatctcctgttcttcaggtgctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccctattacaacatcctgctctattccttggtgccacttatgttaattgcggggattgtcatttgtgcattttgggtgtacaggcatcacaagatggcctaccctcctgtacttgttccaactcaagacccaggaccacccccaccttctccattactaggtttgaaaccactgcagttattagaagtgaaagcaaggggaagatttggttgtgtctggaaagcccagttgcttaacgaatatgtggctgtcaaaatatttccaatacaggacaaacagtcatggcaaaatgaatacgaagtctacagtttgcctggaatgaagcatgagaacatattacagttcattggtgcagaaaaacgaggcaccagtgttgatgtggatctttggctgatcacagcatttcatgaaaagggttcactatcagactttcttaaggctaatgtggtctcttggaatgaactgtgtcatattgcagaaaccatggctagaggattggcatatttacatgaggatatacctggcctaaaagatggccacaaacctgccatatctcacagggacatcaaaagtaaaaatgtgctgttgaaaaacaacctgacagcttgcattgctgactttgggttggccttaaaatttgaggctggcaagtctgcaggcgatacccatggacaggttggtacccggaggtacatggctccagaggtattagagggtgctataaacttccaaagggatgcatttttgaggatagatatgtatgccatgggattagtcctatgggaactggcttctcgctgtactgctgcagatggacctgtagatgaatacatgttgccatttgaggaggaaattggccagcatccatctcttgaagacatgcaggaagttgttgtgcataaaaaaaagaggcctgttttaagagattattggcagaaacatgctggaatggcaatgctctgtgaaaccattgaagaatgttgggatcacgacgcagaagccaggttatcagctggatgtgtaggtgaaagaattacccagatgcagagactaacaaatattattaccacagaggacattgtaacagtggtcacaatggtgacaaatgttgactttcctcccaaagaatctagtcta

SEQ ID NO: 13 - DNA, 345 bp, Homo sapiens
atacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccc

SEQ ID NO: 14 - protein, 225 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 15 - protein, 225 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 16 - protein, 225 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 225 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 18 - protein, 225 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 225 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 20 - protein, 228 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGK

SEQ ID NO: 21 - protein, 228 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPPSEELALNELVTLTCLVKGFYPSDIAVEWESNGQELPREKYLTWAPVLDSDGSFFLYSILRVAAEDWKKGDTFSCSVMHEALHNHYTQKSLDRSPGK

SEQ ID NO: 22 - protein, 225 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 223 aa, Homo sapiens
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 24 - protein, 232 aa, Homo sapiens
EPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 25 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 26 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 261 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSAQLEKELQALEKENAQLEWELQALEKELAQGAT

SEQ ID NO: 28 - protein, 261 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSAQLKKKLQALKKKNAQLKWKLQALKKKLAQGAT

SEQ ID NO: 29 - protein, 5 aa, Artificial Sequence
  Description of Artificial Sequence: Synthetic peptide
GGGGS

SEQ ID NO: 30 - protein, 10 aa, Artificial Sequence
  Description of Artificial Sequence: Synthetic peptide
  See specification as filed for detailed description of substitutions and preferred embodiments
GGGGSGGGGS

SEQ ID NO: 31 - protein, 3 aa, Artificial Sequence
  Description of Artificial Sequence: Synthetic peptide
GGG

SEQ ID NO: 32 - protein, 4 aa, Artificial Sequence
  Description of Artificial Sequence: Synthetic peptide
GGGG

SEQ ID NO: 33 - protein, 10 aa, Artificial Sequence
  Description of Artificial Sequence: Synthetic peptide
GGGGSGGGGS

SEQ ID NO: 34 - protein, 4 aa, Artificial Sequence
  Description of Artificial Sequence: Synthetic peptide
SGGG

SEQ ID NO: 35 - protein, 5 aa, Artificial Sequence
  Description of Artificial Sequence: Synthetic peptide
SGGGG

SEQ ID NO: 36 - protein, 4 aa, Artificial Sequence
  Description of Artificial Sequence: Synthetic peptide
TGGG

SEQ ID NO: 37 - protein, 5 aa, Artificial Sequence
  Description of Artificial Sequence: Synthetic peptide
TGGGG

SEQ ID NO: 38 - protein, 11 aa, Artificial Sequence
  Description of Artificial Sequence: Synthetic peptide
TGGGGSGGGGS

SEQ ID NO: 39 - protein, 16 aa, Artificial Sequence
  Description of Artificial Sequence: Synthetic peptide
TGGGGSGGGGSGGGGS

SEQ ID NO: 40 - protein, 21 aa, Artificial Sequence
  Description of Artificial Sequence: Synthetic peptide
TGGGGSGGGGSGGGGSGGGGS

SEQ ID NO: 41 - protein, 26 aa, Artificial Sequence
  Description of Artificial Sequence: Synthetic peptide
TGGGGSGGGGSGGGGSGGGGSGGGGS

SEQ ID NO: 42 - protein, 31 aa, Artificial Sequence
  Description of Artificial Sequence: Synthetic polypeptide
TGGGGSGGGGSGGGGSGGGGSGGGGSGGGGS

SEQ ID NO: 43 - protein, 36 aa, Artificial Sequence
  Description of Artificial Sequence: Synthetic polypeptide
TGGGGSGGGGSGGGGSGGGGSGGGGSGGGGSGGGGS

SEQ ID NO: 44 - protein, 10 aa, Artificial Sequence
  Description of Artificial Sequence: Synthetic peptide
TGGGPKSCDK

SEQ ID NO: 45 - protein, 22 aa, Artificial Sequence
  Description of Artificial Sequence: Synthetic peptide
MDAMKRGLCCVLLLCGAVFVSP

SEQ ID NO: 46 - protein, 368 aa, Artificial Sequence
  Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 47 - DNA, 1104 bp, Artificial Sequence
  Description of Artificial Sequence: Synthetic polynucleotide
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccggaaggagatgaccaagaaccaggtcagcctgacctgcctggtcaaaggcttctatcccagcgacatcgccgtggagtgggagagcaatgggcagccggagaacaactacaagaccacgcctcccgtgctgaagtccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 48 - protein, 343 aa, Artificial Sequence
  Description of Artificial Sequence: Synthetic polypeptide
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 49 - protein, 262 aa, Artificial Sequence
  Description of Artificial Sequence: Synthetic polypeptide
MDAMKRGLCCVLLLCGAVFVSPGASNTKVDKRVTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 50 - DNA, 786 bp, Artificial Sequence
  Description of Artificial Sequence: Synthetic polynucleotide
atggatgcaatgaagagagggctctgctgtgtgctgctgctgtgtggagcagtcttcgtttcgcccggcgccagcaacaccaaggtggacaagagagttaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctatagcgacctcaccgtggacaagagcaggtggcagcaggggaacgtcttctcatgctccgtgatgcatgaggctctgcacaaccactacacgcagaagagcctctccctgtctccgggtaaa

SEQ ID NO: 51 - protein, 238 aa, Artificial Sequence
  Description of Artificial Sequence: Synthetic polypeptide
SNTKVDKRVTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 52-54 - skipped (3 entries)

SEQ ID NO: 55 - protein, 369 aa, Artificial Sequence
  Description of Artificial Sequence: Synthetic polypeptide
MDAMKRGLCCVLLLCGAVFVSPGAAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 56 - DNA, 1107 bp, Artificial Sequence
  Description of Artificial Sequence: Synthetic polynucleotide
atggatgcaatgaagagagggctctgctgtgtgctgctgctgtgtggagcagtcttcgtttcgcccggcgccgctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttctgttgctgtgagggcaatatgtgtaatgaaaagttttcttattttccggagatggaagtcacacagcccacttcaaatccagttacacctaagccacccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccggaaggagatgaccaagaaccaggtcagcctgacctgcctggtcaaaggcttctatcccagcgacatcgccgtggagtgggagagcaatgggcagccggagaacaactacaagaccacgcctcccgtgctgaagtccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 57 - protein, 344 aa, Artificial Sequence
  Description of Artificial Sequence: Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 58 - protein, 369 aa, Artificial Sequence
  Description of Artificial Sequence: Synthetic polypeptide
MDAMKRGLCCVLLLCGAVFVSPGAAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 344 aa, Artificial Sequence
  Description of Artificial Sequence: Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 368 aa, Artificial Sequence
  Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 343 aa, Artificial Sequence
  Description of Artificial Sequence: Synthetic polypeptide
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 262 aa, Artificial Sequence
  Description of Artificial Sequence: Synthetic polypeptide
MDAMKRGLCCVLLLCGAVFVSPGASNTKVDKRVTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 63 - protein, 238 aa, Artificial Sequence
  Description of Artificial Sequence: Synthetic polypeptide
SNTKVDKRVTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 64-65 - skipped (2 entries)

SEQ ID NO: 66 - protein, 115 aa, Homo sapiens; identical to SEQ ID NO: 10

SEQ ID NO: 67 - protein, 150 aa, Rattus sp.
MTAPWAALALLWGSLCAGSGRGEAETRECIYYNANWELERTNQSGLERCEGEQDKRLHCYASWPNSSGTIELVKKGCWLDDFNCYDRQECVATEENPQVYFCCCEGNFCNERFTHLPEPGGPEVTYEPPPTAPTLLTVLAYSLLPIGGLS

SEQ ID NO: 68 - protein, 150 aa, Sus sp.
MTAPWAALALLWGSLCVGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLS

SEQ ID NO: 69 - protein, 150 aa, Mus sp.
MTAPWAALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEPGGPEVTYEPPPTAPTLLTVLAYSLLPIGGLS

SEQ ID NO: 70 - protein, 150 aa, Homo sapiens
MTAPWVALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLS

SEQ ID NO: 71 - protein, 150 aa, Bos taurus
MTAPWAALALLWGSLCAGSGRGEAETRECIYYNANWELERTNQSGLERCEGERDKRLHCYASWRNSSGTIELVKKGCWLDDFNCYDRQECVATEENPQVYFCCCEGNFCNERFTHLPEAGGPEVTYEPPPTAPTLLTVLAYSLLPVGGLS

SEQ ID NO: 72 - protein, 150 aa, Xenopus sp.
MGASVALTFLLLLATFRAGSGHDEVETRECIYYNANWELEKTNQSGVERLVEGKKDKRLHCYASWRNNSGFIELVKKGCWLDDFNCYDRQECIAKEENPQVFFCCCEGNYCNKKFTHLPEVETFDPKPQPSASVLNILIYSLLPIVGLSM

SEQ ID NO: 73 - protein, 150 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLI

SEQ ID NO: 74 - protein, 154 aa, Artificial Sequence
  Description of Artificial Sequence: Synthetic polypeptide
  MOD_RES (8)..(8): T, A, or not present
  MOD_RES (121)..(121): P, A, V, or M
MTAPWAAXLALLWGSLCAGSGRGEAETRECIYYNANWELERTNQSGIERLCEGEQDKRLHCYASWRNSSGTLEIVKKGCWLDDFNCYDRQECVATEENPQVYFCCCEGNFCNERFTHLPEXGGPEVTYEPKPPTAPTLLTVLAYSLLPIGGLSM

SEQ ID NO: 75 - protein, 115 aa, Homo sapiens; identical to SEQ ID NO: 10

SEQ ID NO: 76 - protein, 115 aa, Ovis aries
ILGRSETQECIFYNANWERDRTNRTGVESCYGDKDKRRHCFATWKNISGSIDIVKQGCWLDDINCYDRTDCIEKKDSPEVYFCCCEGNMCNERFSYFPEMEVTQPTSNPVTPKPP

SEQ ID NO: 77 - protein, 115 aa, Unknown
  Description of Unknown: Talpidae family peptide sequence
ILGRSETQECLFFNANWERDRTNQTGVEPCYGDKDKRRHCFATWKNISGSIEIVKQGCWLDDINCYDRTDCIEKKDSPEVYFCCCEGNMCNEKFSYFPEMEVTQPTSNPVTPKAP

SEQ ID NO: 78 - protein, 115 aa, Mus sp.
ILGRSETQECLFFNANWERDRTNQTGVEPCYGDKDKRRHCFATWKNISGSIEIVKQGCWLDDINCYDRTDCIEKKDSPEVYFCCCEGNMCNEKFSYFPEMEVTQPTSNPVTPKPP

SEQ ID NO: 79 - protein, 115 aa, Gallus gallus
ILGRSETQECIYYNANWEKDKTNRSGIEPCYGDKDKRRHCFATWKNISGSIEIVKQGCWLDDINCYDRNDCIEKKDSPEVFFCCCEGNMCNERFFYFPEMEVTQPTSNPVTPKPP

SEQ ID NO: 80 - protein, 115 aa, Bos taurus
ILGRSETQECIFYNANWERDRTNRTGVESCYGDKDKRRHCFATWKNISGSIEIVKQGCWLDDINCYDRTDCIEKKDSPEVYFCCCEGNMCNERFSYFPEMEVTQPTSNPVTPKPP

SEQ ID NO: 81 - protein, 115 aa, Tyto alba; identical to SEQ ID NO: 79

SEQ ID NO: 82 - protein, 115 aa, Myotis davidii
ILGRSETQECIFYNANWERDKTNRTGVELCYGDKDKRRHCFATWKNISGSIEIVKQGCWLDDINCYDRTDCIEKKDSPEVYFCCCEGNMCNERFSYFPEMEVTQPTSNPVTPKPP

SEQ ID NO: 83 - protein, 107 aa, Artificial Sequence
  Description of Artificial Sequence: Synthetic polypeptide
ETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPT

SEQ ID NO: 84 - protein, 6 aa, Artificial Sequence
  Description of Artificial Sequence: Synthetic 6xHis tag
HHHHHH

(19) United States
(12) Patent Application Publication
(10) Pub. No.: US 2023/0190875 A1
(43) Pub. Date: Jun. 22, 2023

(54) SINGLE-ARM ACTRIIA AND ACTRIIB HETEROMULTIMERS AND METHODS FOR TREATING PULMONARY HYPERTENSION

(71) Applicant: Acceleron Pharma Inc., Cambridge, MA (US)

(72) Inventors: Ravindra Kumar, Acton, MA (US); Gang Li, Sudbury, MA (US)

(21) Appl. No.: 17/784,029

(22) PCT Filed: Dec. 9, 2020
(86) PCT No.: PCT/US2020/064026

§ 371 Date: Jun. 9, 2022

Related U.S. Application Data
(60) Provisional application No. 62/946,089, filed on Dec. 10, 2019.

(51) Int. Cl. A61K 38/17; A61P 9/12; A61K 45/06

(52) U.S. Cl. CPC A61K 38/179; A61K 38/1796; A61K 45/06; A61P 9/12

(57) ABSTRACT

In some aspects, the disclosure relates to single-arm ActRIIA heteromultimers and single-arm ActRIIB heteromultimers and methods of using such heteromultimers to treat, prevent, or reduce the progression rate and/or severity of pulmonary hypertension (PH), particularly treating, preventing or reducing the progression rate and/or severity of one or more PH-associated complications. The disclosure also provides methods of using a single-arm ActRIIA heteromultimer or single-arm ActRIIB heteromultimer to treat, prevent, or reduce the progression rate and/or severity of a variety of conditions including, but not limited to, pulmonary vascular remodeling, pulmonary fibrosis, and right ventricular hypertrophy. The disclosure further provides methods of using single-arm ActRIIA heteromultimers and single-arm ActRIIB heteromultimers to reduce right ventricular systolic pressure in a subject in need thereof.

### CROSS-REFERENCE TO RELATED APPLICATIONS

This application claims the benefit of and priority to U.S. Provisional Application No. 62/946,089, filed Dec. 10, 2019. The foregoing application is incorporated herein by reference in its entirety.

### BACKGROUND OF THE INVENTION

Pulmonary hypertension (PH) is a disease characterized by high blood pressure in lung vasculature, including pulmonary arteries, pulmonary veins, and pulmonary capillaries. In general, PH is defined as a mean pulmonary arterial (PA) pressure ≧25 mm Hg at rest or ≧30 mm Hg with exercise [Hill et al., Respiratory Care 54(7):958-68 (2009)]. The main PH symptom is difficulty in breathing or shortness of breath, and other symptoms include fatigue, dizziness, fainting, peripheral edema (swelling in foot, legs or ankles), bluish lips and skin, chest pain, angina pectoris, light-headedness during exercise, non-productive cough, racing pulse and palpitations. PH can be a severe disease causing heart failure, which is one of the most common causes of death in people who have pulmonary hypertension. Postoperative pulmonary hypertension may complicate many types of surgeries or procedures, and present a challenge associated with a high mortality.

PH may be grouped based on different manifestations of the disease sharing similarities in pathophysiologic mechanisms, clinical presentation, and therapeutic approaches [Simonneau et al., JACC 54(1):S44-54 (2009)]. Clinical classification of PH was first proposed in 1973, and a recent updated clinical classification was endorsed by the World Health Organization (WHO) in 2008. According to the updated PH clinical classification, there are five main groups of PH: pulmonary arterial hypertension (PAH), characterized by a PA wedge pressure ≧15 mm Hg; PH owing to a left heart disease (also known as pulmonary venous hypertension or congestive heart failure), characterized by a PA wedge pressure >15 mm Hg; PH owing to lung diseases and/or hypoxia; chronic thromboemboli PH; and PH with unclear or multifactorial etiologies [Simonneau et al., JACC 54(1):S44-54 (2009); Hill et al., Respiratory Care 54(7):958-68 (2009)]. PAH is further classified into idiopathic PAH (IPAH), a sporadic disease in which there is neither a family history of PAH nor an identified risk factor; heritable PAH; PAH induced by drugs and toxins; PAH associated with connective tissue diseases, HIV infection, portal hypertension, congenital heart diseases, schistosomiasis, and chronic hemolytic anemia; and persistent PH of newborns [Simonneau et al., JACC 54(1):S44-54 (2009)]. Diagnosis of various types of PH requires a series of tests. In general, PH treatment depends on the cause or classification of the PH. Where PH is caused by a known medicine or medical condition, it is known as a secondary PH, and its treatment is usually directed at the underlying disease. Treatment of pulmonary venous hypertension generally involves optimizing left ventricular function by administering diuretics, beta blockers, and ACE inhibitors, or repairing or replacing a mitral valve or aortic valve. PAH therapies include pulmonary vasodilators, digoxin, diuretics, anticoagulants, and oxygen therapy. Pulmonary vasodilators target different pathways, including prostacyclin pathway (e.g., prostacyclins, including intravenous epoprostenol, subcutaneous or intravenous treprostinil, and inhaled iloprost), nitric oxide pathway (e.g., phosphodiesterase-5 inhibitors, including sildenafil and tadalafil), and endotheline-1 pathway (e.g., endothelin receptor antagonists, including oral bosentan and oral ambrisentan) [Humbert, M. Am. J. Respir. Crit. Care Med. 179:650-6 (2009); Hill et al., Respiratory Care 54(7):958-68 (2009)]. However, current therapies provide no cure for PH, and they do not directly treat the underling vascular remodeling and muscularization of blood vessels observed in many PH patients. Thus, there is a high, unmet need for effective therapies for treating pulmonary hypertension. Accordingly, it is an object of the present disclosure to provide methods for treating, preventing, or reducing the progression rate and/or severity of PH, particular treating, preventing or reducing the progression rate and/or severity of one or more PH-associated complications.

### SUMMARY OF THE INVENTION

In part, the disclosure provides single-arm heteromultimeric complexes comprising a single ActRIIA or a single ActRIIB polypeptide, including fragments and variants thereof. These constructs may be referred to herein as single-arm heteromultimers, a single-arm ActRIIA heteromultimer or heterodimer, and single-arm ActRIIB heteromultimer or heterodimer. Optionally, single-arm polypeptide heteromultimers disclosed herein (e.g., a single-arm ActRIIB heteromultimer, such as a single-arm ActRIIB heterodimer Fc fusion) have different ligand-binding specificities/profiles compared to a corresponding heteromultimer (e.g., an ActRIIB homodimer Fc fusion). Novel properties are exhibited by heteromultimers comprising a single domain of an ActRIIA or a single domain of an ActRIIB polypeptide, as shown by Examples herein.

Heteromultimeric structures include, for example, heterodimers, heterotrimers, and higher order complexes. Preferably, ActRIIA or ActRIIB polypeptides as described herein comprise a ligand-binding domain of the receptor, for example, an extracellular domain of an ActRIIA or ActRIIB receptor. Accordingly, in certain aspects, heteromultimers described herein comprise an extracellular domain of an ActRIIA or ActRIIB polypeptide, as well as truncations and variants thereof. Preferably, ActRIIA or ActRIIB polypeptides as described herein, as well as heteromultimers comprising the same, are soluble. In certain aspects, heteromultimers of the disclosure bind to one or more ActRIIA or ActRIIB ligands (e.g., activin A, activin B, GDF11, GDF8, GDF3, BMP5, BMP6, and BMP10. Optionally, protein complexes of the disclosure bind to one or more of these ligands with a K_{D} of less than or equal to 10⁻⁸, 10⁻⁹, 10⁻¹⁰, 10⁻¹¹, or 10⁻¹². In general, single-arm heteromultimers of the disclosure antagonize (inhibit) one or more activities of at least one ActRIIA or ActRIIB ligand, and such alterations in activity may be measured using various assays known in the art, including, for example, a cell-based assay as described herein. Preferably, single-arm heteromultimers of the disclosure exhibit a serum half-life of at least 4, 6, 12, 24, 36, 48, or 72 hours in a mammal (e.g., a mouse or a human). Optionally, single-arm heteromultimers of the disclosure may exhibit a serum half-life of at least 6, 8, 10, 12, 14, 20, 25, or 30 days in a mammal (e.g., a mouse or a human).

In part, the disclosure provides ActRIIA or ActRIIB single-arm heteromultimers that can be used to treat pulmonary diseases or conditions (e.g., pulmonary hypertension, (PH), pulmonary arterial hypertension (PAH), idiopathic pulmonary fibrosis (IPF), interstitial lung disease (ILD)). Positive effects were observed for a single-arm ActRIIB heteromultimer in the Sugen hypoxia PAH model. The disclosure establishes that antagonists of the ActRII (e.g., ActRIIA and ActRIIB) signaling pathways may be used to reduce the severity of pulmonary hypertension, and that desirable therapeutic agents may be selected on the basis of ActRII signaling antagonist activity. Therefore, in some embodiments, the disclosure provides methods for using various single-arm ActRIIA heteromultimers or single-arm ActRIIB heteromultimers for treating pulmonary diseases or conditions, including but not limited to hypertension, particularly pulmonary hypertension and pulmonary arterial hypertension, idiopathic pulmonary fibrosis (IPF), and interstitial lung disease (ILD), including, for example, single-arm heteromultimers that inhibit one or more ActRIIA or ActRIIB ligands [e.g., activin A, activin B, GDF11, GDF8, GDF3, BMP6, BMP5, and BMP10].

In some embodiments, the present disclosure provides methods of treating pulmonary diseases or conditions, comprising administering a single-arm ActRIIB heteromultimer to a subject in need thereof. In some embodiments, the present disclosure provides methods of treating pulmonary hypertension (PH) comprising administering a single-arm ActRIIB heteromultimer to a subject in need thereof, the heteromultimer comprising a first polypeptide covalently or non-covalently associated with a second polypeptide, wherein the first polypeptide comprises the amino acid sequence of a first member of an interaction pair and the amino acid sequence of ActRIIB; and the second polypeptide comprises the amino acid sequence of a second member of the interaction pair, and wherein the second polypeptide does not comprise ActRIIB.

In some embodiments, the ActRIIB polypeptide comprises, consists, or consists essentially of an amino acid sequence that is: at least 70%, 80%, 85%, 90%, 91%, 92%, 93%, 94% 95%, 96%, 97%, 98%, 99% or 100% identical to the sequence of any of SEQ ID Nos: 1, 2, 3, 4, 5, and 6; or at least 70%, 80%, 85%, 90%, 91%, 92%, 93%, 94% 95%, 96%, 97%, 98%, 99% or 100% identical to a polypeptide that begins at any one of amino acids 20, 21, 22, 23, 24, 25, 26, 27, 28, or 29 of SEQ ID NO: 1, and ends at any one of amino acids 109, 110, 111, 112, 113, 114, 115, 116, 117, 118, 119, 120, 121, 122, 123, 124, 125, 126, 127, 128, 129, 130, 131, 132, 133, or 134 of SEQ ID NO: 1.

In some embodiments, an ActRIIB polypeptide may comprise an amino acid sequence that is at least 70%, 75%, 80%, 85%, 90%, 91%, 92%, 93%, 94%, 95%, 96%, 97%, 98%, 99%, or 100% identical to the amino acid sequence of SEQ ID NO: 79. In some embodiments, an ActRIIB polypeptide may comprise an amino acid sequence that is at least 70%, 75%, 80%, 85%, 90%, 91%, 92%, 93%, 94%, 95%, 96%, 97%, 98%, 99%, or 100% identical to the amino acid sequence of SEQ ID NO: 79, wherein the ActRIIB polypeptide comprises an acidic amino acid at position 79 with respect to SEQ ID NO: 1. In certain embodiments, ActRIIB polypeptides to be used in accordance with the methods and uses described herein do not comprise an acidic amino acid at the position corresponding to L79 of SEQ ID NO: 1. In certain embodiments, ActRIIB polypeptides to be used in accordance with the methods and uses described herein do not comprise an aspartic acid (D) at the position corresponding to L79 of SEQ ID NO: 1.

In some embodiments, the present disclosure provides methods of treating pulmonary diseases or conditions, comprising administering a single-arm ActRIIA heteromultimer to a subject in need thereof. In some embodiments, the present disclosure provides methods of treating pulmonary hypertension (PH) comprising administering a single-arm ActRIIA heteromultimer to a subject in need thereof, the heteromultimer comprising a first polypeptide covalently or non-covalently associated with a second polypeptide, wherein the first polypeptide comprises the amino acid sequence of a first member of an interaction pair and the amino acid sequence of ActRIIA; and the second polypeptide comprises the amino acid sequence of a second member of the interaction pair, and wherein the second polypeptide does not comprise ActRIIA.

In some embodiments, the ActRIIA polypeptide comprises, consists, or consists essentially of an amino acid sequence that is at least 70%, 80%, 85%, 90%, 91%, 92%, 93%, 94% 95%, 96%, 97%, 98%, 99% or 100% identical to the sequence of any of SEQ ID Nos: 9, 10, and 11; or at least 70%, 80%, 85%, 90%, 91%, 92%, 93%, 94% 95%, 96%, 97%, 98%, 99% or 100% identical to a polypeptide that begins at any one of amino acids 21, 22, 23, 24, 25, 26, 27, 28, 29, or 30 of SEQ ID NO: 9, and ends at any one of amino acids 110, 111, 112, 113, 114, 115, 116, 117, 118, 119, 120, 121, 122, 123, 124, 125, 126, 127, 128, 129, 130, 131, 132, 133, 134 or 135 of SEQ ID NO: 9.

In some embodiments of the present disclosure, the heteromultimer is a heterodimer.

In some embodiments of the present disclosure, the first member of an interaction pair comprises a first constant region from an IgG heavy chain. In some embodiments, the first constant region from an IgG heavy chain is a first immunoglobulin Fc domain. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence selected from any one of SEQ ID NOs: 14-28.

In some embodiments of the present disclosure, the second member of an interaction pair comprises a second constant region from an IgG heavy chain. In some embodiments, the second constant region from an IgG heavy chain is a first immunoglobulin Fc domain. In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence selected from any one of SEQ ID NOs: 14-28.

In some embodiments of the present disclosure, the first polypeptide comprises an amino acid sequence that is at least 70%, 80%, 85%, 90%, 91%, 92%, 93%, 94% 95%, 96%, 97%, 98%, 99% or 100% identical to a sequence selected from any one of SEQ ID NOs: 46, 48, 55, 57, 58, 59, 60, and 61. In some embodiments, the second polypeptide comprises an amino acid sequence that is at least 70%, 80%, 85%, 90%, 91%, 92%, 93%, 94% 95%, 96%, 97%, 98%, 99% or 100% identical to a sequence selected from any one of SEQ ID NOs: 49, 51, 62, and 63.

In some embodiments of the present disclosure, a single-arm ActRIIB heteromultimer comprises a linker domain positioned between the ActRIIB polypeptide and the first member of an interaction pair. In some embodiments, the linker domain comprises an amino acid sequence selected from any one of SEQ ID NOs: 29-44.

In some embodiments of the present disclosure, a single-arm ActRIIA heteromultimer comprises a linker domain positioned between the ActRIIA polypeptide and the first member of an interaction pair. In some embodiments, the linker domain comprises an amino acid sequence selected from any one of SEQ ID NOs: 29-44.

In some embodiments of the present disclosure, the first polypeptide and/or second polypeptide comprises one or more modified amino acid residues selected from: a glycosylated amino acid, a PEGylated amino acid, a famesylated amino acid, an acetylated amino acid, a biotinylated amino acid, an amino acid conjugated to a lipid moiety, and an amino acid conjugated to an organic derivatizing agent. In some embodiments, the first polypeptide and/or second polypeptide is glycosylated and has a glycosylation pattern obtainable from expression of the first polypeptide and/or second polypeptide in a CHO cell.

In some embodiments, the heteromultimer (e.g., heterodimer Fc fusion) binds to one or more of ActRIIA or ActRIIB ligands selected from the group consisting of activin A, activin B, GDF11, GDF8, GDF3, BMP5, BMP6, and BMP10. In some embodiments, a single-arm ActRIIB heterodimer Fc fusion has greater ligand selectivity than an ActRIIB homodimer Fc fusion. In some embodiments, an ActRIIB homodimer Fc fusion binds strongly to activin A, activin B, GDF11, GDF8, and BMP10. In some embodiments, a single-arm ActRIIB heterodimer Fc fusion binds strongly to activin B and GDF11 and binds intermediately to GDF8 and activin A. In some embodiments, a single-arm ActRIIB heterodimer Fc fusion displays weak, minimal, or undetectable binding to BMP10. In some embodiments, a single-arm ActRIIB heterodimer Fc fusion antagonizes activin A, activin B, GDF8, and GDF11 and minimally antagonizes one or more of BMP9, BMP10, BMP6, and GDF3. In some embodiments, single-arm ActRIIA heterodimer Fc fusion exhibits preferential binding to activin A over activin B combined with greatly enhanced selectivity for activin A over GDF11. In some embodiments, a single-arm ActRIIA heterodimer Fc fusion largely retains intermediate binding to GDF8 and BMP10 as observed with an ActRIIA homodimer Fc fusion. In some embodiments, a single-arm ActRIIA heterodimer Fc fusion is utilized in therapeutic applications where it is desirable to antagonize activin A preferentially over activin B while minimizing antagonism of GDF11. In some embodiments of the present disclosure, a single-arm ActRIIA heterodimer Fc fusion or a single-arm ActRIIB heterodimer Fc fusion inhibits the activity of one or more ActRIIA or ActRIIB ligands in a cell-based assay.

In some embodiments of the present disclosure, the pulmonary hypertension is pulmonary arterial hypertension. In some embodiments, methods of the present disclosure comprise further administering to the subject an additional active agent and/or supportive therapy for treating pulmonary hypertension. In some embodiments, the additional active agent and/or supportive therapy for treating pulmonary hypertension is selected from the group consisting of: prostacyclin and derivatives thereof (e.g., epoprostenol, treprostinil, and iloprost); prostacyclin receptor agonists (e.g., selexipag); endothelin receptor antagonists (e.g., thelin, ambrisentan, macitentan, and bosentan); calcium channel blockers (e.g., amlodipine, diltiazem, and nifedipine; anticoagulants (e.g., warfarin); diuretics; oxygen therapy; atrial septostomy; pulmonary thromboendarterectomy; phosphodiesterase type 5 inhibitors (e.g., sildenafil and tadalafil); activators of soluble guanylate cyclase (e.g., cinaciguat and riociguat); ASK-1 inhibitors (e.g., CIIA; SCH79797; GS-4997; MSC2032964A; 3H-naphtho[1,2,3-de]quiniline-2,7-diones, NQDI-1; 2-thioxo-thiazolidines, 5-bromo-3-(4-oxo-2-thioxo-thiazolidine-5-ylidene)-1,3-dihydro-indol-2-one); NF-κB antagonists (e.g., dh404, CDDO-epoxide; 2.2-difluoropropionamide; C28 imidazole (CDDO-Im); 2-cyano-3,12-dioxoolean-1,9-dien-28-oic acid (CDDO); 3-Acetyloleanolic Acid; 3-Triflouroacetyloleanolic Acid; 28-Methyl-3-acetyloleanane; 28-Methyl-3-trifluoroacetyloleanane; 28-Methyloxyoleanolic Acid; SZC014; SCZ015; SZC017; PEGylated derivatives of oleanolic acid; 3-O-(beta-D-glucopyranosyl) oleanolic acid; 3-O- [beta-D-glucopyranosyl-(1-->3)-beta-D-glucopyranosyl] oleanolic acid; 3-O-[beta-D-glucopyranosyl-(1-->2)-beta-D-glucopyranosyl] oleanolic acid; 3-O-[beta-D-glucopyranosyl-(1-->3)-beta-D-glucopyranosyl] oleanolic acid 28-O-beta-D-glucopyranosyl ester; 3-O-[beta-D-glucopyranosyl-(1-->2)-beta-D-glucopyranosyl] oleanolic acid 28-O-beta-D-glucopyranosyl ester; 3-O-[a-L-rhamnopyranosyl-(1-->3)-beta-D-glucuronopyranosyl] oleanolic acid; 3-O-[alpha-L-rhamnopyranosyl-(1-->3)-beta-D-glucuronopyranosyl] oleanolic acid 28-O-beta-D-glucopyranosyl ester; 28-O-β-D-glucopyranosyl-oleanolic acid; 3-O-β-D-glucopyranosyl (1→3)-β-D-glucopyranosiduronic acid (CS1); oleanolic acid 3-O-β-D-glucopyranosyl (1→3)-β-D-glucopyranosiduronic acid (CS2); methyl 3,11-dioxoolean-12-en-28-olate (DIOXOL); ZCVI₄-2; Benzyl 3-dehydr-oxy-1,2,5-oxadiazolo[3′,4′:2,3]oleanolate); lung and/or heart transplantation.

In some embodiments the subject has a resting pulmonary arterial pressure (PAP) of at least 25 mm Hg (e.g., 25, 30, 35, 40, 45, or 50 mm Hg). In some embodiments, methods of the present disclosure reduces PAP in the subject. In some embodiments, the PAP is reduced by at least 3 mmHg (e.g., at least 3, 5, 7, 10, 12, 15, 20, or 25 mm Hg) in the subject.

In some embodiments, methods of the present disclosure reduce pulmonary vascular resistance in the subject. In some embodiments, pulmonary capillary wedge pressure is increased. In some embodiments, left ventricular end-diastolic pressure is increased.

In some embodiments, methods of the present disclosure increase exercise capacity of the subject. In some embodiments, methods of the present disclosure increase the subject’s 6-minute walk distance. In some embodiments, the subject’s 6-minute walk distance is increased by at least 10 meters (e.g., at least 10, 20, 30, 40, 50, 60, 70, 80, 90, 100 or more meters). In some embodiments, the subject has a 6-minute walk distance from 150 to 400 meters. In some embodiments, the subject’s 6-minute walk distance is increased by at least 10 meters (e.g., at least 10, 20, 30, 40, 50, 60, 70, 80, 90, 100, 125, 150, 175, 200, 250, 300, or more than 400 meters).

In some embodiments, methods of the present disclosure reduce the subject’s Borg dyspnea index (BDI). In some embodiments, the subject’s BDI is reduced by at least 0.5 index points (e.g., at least 0.5, 1, 1.5, 2, 2.5, 3, 3.5, 4, 4.5, 5, 5.5, 6, 6.5, 7, 7.5, 8, 8.5, 9, 9.5, or 10 index points).

In some embodiments, the subject has Functional Class I, Class II, Class III, or Class IV pulmonary hypertension as recognized by the World Health Organization. In some embodiments method of the present disclosure prevent or delay pulmonary hypertension Functional Class progression (e.g., prevent or delay progression from Functional Class I to Class II, Class II to Class III, or Class III to Class IV pulmonary hypertension as recognized by the World Health Organization). In some embodiments, methods of the present disclosure promote or increase pulmonary hypertension Functional Class regression (e.g., promote or increase regression from Class IV to Class III, Class III to Class II, or Class II to Class I pulmonary hypertension as recognized by the World Health Organization).

In some embodiments, methods of the present disclosure decrease pulmonary arterial pressure in the subject. In some embodiments, pulmonary arterial pressure in the subject is decreased by at least 10% (e.g., 10%, 15%, 20%, 25%, 30%, 35%, 40%, 45%, 50%, 55%, 60%, 65%, 70%, 75%, or at least 80%).

In some embodiments, methods of the present disclosure decrease ventricle hypertrophy in the subject. In some embodiments, ventricle hypertrophy is decreased in the subject by at least 10% (e.g., 10%, 15%, 20%, 25%, 30%, 35%, 40%, 45%, 50%, 55%, 60%, 65%, 70%, 75%, or at least 80%).

In some embodiments, methods of the present disclosure decrease smooth muscle hypertrophy in the subject. In some embodiments, smooth muscle hypertrophy is decreased in the subject by at least 10% (e.g., 10%, 15%, 20%, 25%, 30%, 35%, 40%, 45%, 50%, 55%, 60%, 65%, 70%, 75%, or at least 80%).

In some embodiments, methods of the present disclosure decrease pulmonary arteriole muscularity in the subject. In some embodiment, pulmonary arteriole muscularity is decreased in the subject by at least 10% (e.g., 10%, 15%, 20%, 25%, 30%, 35%, 40%, 45%, 50%, 55%, 60%, 65%, 70%, 75%, or at least 80%).

In some embodiments, methods of the present disclosure decrease pulmonary vascular resistance in the subject. In some embodiments, pulmonary vascular resistance is decreased in the subject by at least 10% (e.g., 10%, 15%, 20%, 25%, 30%, 35%, 40%, 45%, 50%, 55%, 60%, 65%, 70%, 75%, or at least 80%).

In some embodiments, the subject has pulmonary arterial hypertension and has Functional Class II or Class III pulmonary hypertension in accordance with the World Health Organization’s functional classification system for pulmonary hypertension. In some embodiments, the subject has pulmonary arterial hypertension that is classified as one or more subtypes selected from the group consisting of: idiopathic or heritable pulmonary arterial hypertension, drug- and/or toxin-induced pulmonary hypertension, pulmonary hypertension associated with connective tissue disease, and pulmonary hypertension associated with congenital systemic-to-pulmonary shunts at least 1 year following shunt repair. In some embodiments, the subject has been treated with one or more vasodilators. In some embodiments, the subject has been treated with one or more agents selected from the group consisting of: phosphodiesterase type 5 inhibitors, soluble guanylate cyclase stimulators, prostacyclin receptor agonist, and endothelin receptor antagonists. In some embodiments, the one or more agents is selected from the group consisting of: bosentan, sildenafil, beraprost, macitentan, selexipag, epoprostenol, treprostinil, iloprost, ambrisentan, and tadalafil. In some embodiments, method of the present disclosure further comprise administration of one or more vasodilators. In some embodiments, methods of the present disclosure further comprise administration of one or more agents selected from the group consisting of: phosphodiesterase type 5 inhibitors, soluble guanylate cyclase stimulators, prostacyclin receptor agonist, and endothelin receptor antagonists. In some embodiments, the one or more agents is selected from the group consisting of: bosentan, sildenafil, beraprost, macitentan, selexipag, epoprostenol, treprostinil, iloprost, ambrisentan, and tadalafil.

In some embodiments of the present disclosure, the subject has a hemoglobin level from >8 and <15 g/dl.

In some embodiments, methods of the present disclosure delay clinical worsening of pulmonary hypertension in a subject. In some embodiments, methods of the present disclosure delay clinical worsening of pulmonary hypertension in accordance with the World Health Organization’s functional classification system for pulmonary hypertension. In some embodiments, methods of the present disclosure reduce the risk of hospitalization for one or more complications associated with pulmonary hypertension.

### BRIEF DESCRIPTION OF THE DRAWINGS

FIG. 1 shows an alignment of extracellular domains of human ActRIIA (SEQ ID NO: 66) and human ActRIIB (SEQ ID NO: 2) with the residues that are deduced herein, based on composite analysis of multiple ActRIIB and ActRIIA crystal structures, to directly contact ligand indicated with boxes.

FIG. 2 shows a multiple sequence alignment of various vertebrate ActRIIB precursor proteins without their intracellular domains (SEQ ID NOs: 67, 68, 69, 70, 71, 72, respectively) human ActRIIA precursor protein without its intracellular domain (SEQ ID NO: 73), and a consensus ActRII precursor protein (SEQ ID NO: 74).

FIG. 3 shows a multiple sequence alignment of various vertebrate ActRIIA proteins and human ActRIIA (SEQ ID NOs: 75-82).

FIG. 4 shows multiple sequence alignment of Fc domains from human IgG isotypes using Clustal 2.1. Hinge regions are indicated by dotted underline. Double underline indicates examples of positions engineered in IgG1 Fc (SEQ ID NO: 22) to promote asymmetric chain pairing and the corresponding positions with respect to other isotypes IgG2 (SEQ ID NO: 23), IgG3 (SEQ ID NO: 24) and IgG4 (SEQ ID NO: 26).

FIG. 5 shows ligand binding data for a single-arm ActRIIB heterodimer Fc fusion compared to an ActRIIB homodimer Fc fusion. For each protein heteromultimer, ligands are ranked by off-rate (k_{off} or k_{d}), a kinetic constant that correlates well with ligand signaling inhibition, and listed in descending order of binding affinity (ligands bound most tightly are listed at the top). At left, yellow, red, green, and blue lines indicate magnitude of the off-rate constant. Ligands of particular interest are highlighted in bold while others are represented in gray, and solid black lines indicate ligands whose binding to heterodimer is enhanced or unchanged compared with homodimer, whereas dashed lines indicate substantially reduced binding compared with homodimer. As shown, ActRIIB homodimer Fc fusion binds to each of five high affinity ligands with similarly high affinity, whereas single-arm ActRIIB heterodimer Fc fusion discriminates more readily among these ligands. Thus, single-arm ActRIIB heterodimer Fc fusion binds strongly to activin B and GDF11 and with intermediate strength to GDF8 and activin A. In further contrast to ActRIIB homodimer Fc fusion, single-arm ActRIIB heterodimer Fc fusion displays only weak binding to BMP10 and no binding to BMP9. These data indicate that single-arm ActRIIB heterodimer Fc fusion has greater ligand selectivity than homodimeric ActRIIB Fc fusion.

FIG. 6 shows ligand binding data for a single-arm ActRIIA heterodimer Fc fusion compared to ActRIIA homodimer Fc fusion. Format is the same as for FIG. 5. As shown, ActRIIA homodimer Fc fusion exhibits preferential binding to activin B combined with strong binding to activin A and GDF11, whereas single-arm ActRIIA heterodimer Fc fusion has a reversed preference for activin A over activin B combined with greatly enhanced selectivity for activin A over GDF11 (weak binder). These data indicate that single-arm ActRIIA heterodimer Fc fusion has substantially different ligand selectivity than homodimeric ActRIIA Fc fusion.

FIG. 7 shows therapeutic effect of single-arm ActRIIB heterodimer Fc fusion (“sa IIB hd”) in Su/Hx PAH model. Rats were separated into six different treatment groups (5-10 rats per group): 1) control rats (Tris buffered saline administered s.c. as 1 ml/kg, every three days), “Normal”; 2) treatment with semaxanib (200 mg/kg administered s.c. as a single dose daily)/hypoxia and Tris buffered saline (administered s.c. as 1 ml/kg, every three days) (vehicle treatment group), “PBS”; 3) treatment with single-arm ActRIIB heterodimer Fc fusion (3 mg/kg administered s.c. every three days) and semaxanib (200 mg/kg administered s.c. as a single dose daily)/hypoxia, “sa IIB hd 3mpk”; and 4) treatment with single-arm ActRIIB heterodimer Fc fusion (5 mg/kg administered s.c. every three days) and semaxanib (200 mg/kg administered s.c. as a single dose daily)/hypoxia, “sa IIB hd 5mpk”; and 5) treatment with single-arm ActRIIB heterodimer Fc fusion (10 mg/kg administered s.c. every three days) and semaxanib (200 mg/kg administered s.c. as a single dose daily)/hypoxia, “sa IIB hd 10mpk”; and 6) treatment with sildenafil (30 mg/kg administered orally twice daily) and semaxanib (200 mg/kg administered s.c. as a single dose daily)/hypoxia, “Sildenafil”. FIG. 7A shows systolic pulmonary artery pressure (sPAP) and FIG. 7B shows calculated mean pulmonary artery pressure (mPAP). Hypertrophy was assessed, in part, by measuring the weight of and calculating right ventricle (RV)/left ventricle (LV) + septum (S) FIG. 7C. Sildenafil treatment reduced mean pulmonary arterial pressure by 34.8% and decreased right heart hypertrophy by 6.3% compared to “PBS” animals. Single-arm ActRIIB heterodimer Fc fusion treatment was found have greater effects in treating PAH in this model compared to sildenafil. 3mpk, 5mpk, and 10mpk of single-arm ActRIIB heterodimer Fc fusion treatments resulted in a reduction of mean pulmonary arterial pressure by 43.2%, 47.7%, and 52.8% and decreased right heart hypertrophy by 37.8%, 40.2%, and 56.1% respectively, compared to “PBS” animals. Statistical significance (p value) is depicted as * p<0.05, **p<0.01, ***p<0.001, and ****p<0.0001.

### DETAILED DESCRIPTION OF THE INVENTION

### 1. Overview

In part, the present disclosure relates to single-arm heteromultimers comprising an extracellular domain of ActRIIA or an extracellular domain of ActRIIB, methods of making such single-arm heteromultimers, and uses thereof. As described herein, single-arm heteromultimers may comprise an extracellular domain of ActRIIA or ActRIIB. In certain preferred embodiments, heteromultimers of the disclosure have an altered profile of binding to ActRIIA or ActRIIB ligands relative to a corresponding homomultimer complex (e.g., an ActRIIB heterodimer Fc fusion compared to an ActRIIB homodimer Fc fusion).

The TGF-β superfamily is comprised of over 30 secreted factors including TGF-betas, activins, nodals, bone morphogenetic proteins (BMPs), growth and differentiation factors (GDFs), and anti-Mullerian hormone (AMH) [Weiss et al. (2013) Developmental Biology, 2(1): 47-63]. Members of the superfamily, which are found in both vertebrates and invertebrates, are ubiquitously expressed in diverse tissues and function during the earliest stages of development throughout the lifetime of an animal. Indeed, TGF-β superfamily proteins are key mediators of stem cell self-renewal, gastrulation, differentiation, organ morphogenesis, and adult tissue homeostasis. Consistent with this ubiquitous activity, aberrant TGF-beta superfamily signaling is associated with a wide range of human pathologies including, for example, autoimmune disease, cardiovascular disease, fibrotic disease, and cancer.

Ligands of the TGF-beta superfamily (e.g., ligands binding to ActRIIA or ActRIIB) share the same dimeric structure in which the central 3-½ turn helix of one monomer packs against the concave surface formed by the beta-strands of the other monomer. The majority of TGF-beta family members are further stabilized by an intermolecular disulfide bond. This disulfide bonds traverses through a ring formed by two other disulfide bonds generating what has been termed a ‘cysteine knot’ motif [Lin et al. (2006) Reproduction 132: 179-190; and Hinck et al. (2012) FEBS Letters 586: 1860-1870].

TGF-beta superfamily (e.g., ActRIIA or ActRIIB) signaling is mediated by heteromeric complexes of type I and type II serine/threonine kinase receptors, which phosphorylate and activate downstream SMAD proteins (e.g., SMAD proteins 1, 2, 3, 5, and 8) upon ligand stimulation [Massague (2000) Nat. Rev. Mol. Cell Biol. 1:169-178]. These type I and type II receptors are transmembrane proteins, composed of a ligand-binding extracellular domain with cysteine-rich region, a transmembrane domain, and a cytoplasmic domain with predicted serine/threonine kinase specificity. In general, type I receptors mediate intracellular signaling while the type II receptors are required for binding TGF-beta superfamily ligands. Type I and II receptors form a stable complex after ligand binding, resulting in phosphorylation of type I receptors by type II receptors.

The TGF-beta family can be divided into two phylogenetic branches based on the type I receptors they bind and the Smad proteins they activate. One is the more recently evolved branch, which includes, e.g., the TGF-betas, activins, GDF8, GDF9, GDF11, BMP3 and nodal, which signal through type I receptors that activate Smads 2 and 3 [Hinck (2012) FEBS Letters 586:1860-1870]. The other branch comprises the more distantly related proteins of the superfamily and includes, e.g., BMP2, BMP4, BMP5, BMP6, BMP7, BMP8a, BMP8b, BMP9, BMP10, GDF1, GDF5, GDF6, and GDF7, which signal through Smads 1, 5, and 8.

Activins are members of the TGF-beta superfamily and were initially discovered as regulators of secretion of follicle-stimulating hormone, but subsequently various reproductive and non-reproductive roles have been characterized. There are three principal activin forms (A, B, and AB) that are homo/heterodimers of two closely related β subunits (β_{A}β_{A}, β_{B}β_{B}, and β_{A}β_{B}, respectively). The human genome also encodes an activin C and an activin E, which are primarily expressed in the liver, and heterodimeric forms containing β_{C} or β_{E} are also known. In the TGF-beta superfamily, activins are unique and multifunctional factors that can stimulate hormone production in ovarian and placental cells, support neuronal cell survival, influence cell-cycle progress positively or negatively depending on cell type, and induce mesodermal differentiation at least in amphibian embryos [DePaolo et al. (1991) Proc Soc Ep Biol Med. 198:500-512; Dyson et al. (1997) Curr Biol. 7:81-84; and Woodruff (1998) Biochem Pharmacol. 55:953-963]. In several tissues, activin signaling is antagonized by its related heterodimer, inhibin. For example, in the regulation of follicle-stimulating hormone (FSH) secretion from the pituitary, activin promotes FSH synthesis and secretion, while inhibin reduces FSH synthesis and secretion. Other proteins that may regulate activin bioactivity and/or bind to activin include follistatin (FS), follistatin-related protein (FSRP, also known as FLRG or FSTL3), and α₂-macroglobulin.

As described herein, agents that bind to “activin A” are agents that specifically bind to the β_{A} subunit, whether in the context of an isolated β_{A} subunit or as a dimeric complex (e.g., a β_{A}β_{A} homodimer or a β_{A}β_{B} heterodimer). In the case of a heterodimer complex (e.g., a β_{A}β_{B} heterodimer), agents that bind to “activin A” are specific for epitopes present within the β_{A} subunit, but do not bind to epitopes present within the non-β_{A} subunit of the complex (e.g., the β_{B} subunit of the complex). Similarly, agents disclosed herein that antagonize (inhibit) “activin A” are agents that inhibit one or more activities as mediated by a β_{A} subunit, whether in the context of an isolated β_{A} subunit or as a dimeric complex (e.g., a β_{A}β_{A} homodimer or a β_{A}β_{B} heterodimer). In the case of β_{A}β_{B} heterodimers, agents that inhibit “activin A” are agents that specifically inhibit one or more activities of the β_{A} subunit, but do not inhibit the activity of the non-β_{A} subunit of the complex (e.g., the β_{B} subunit of the complex). This principle applies also to agents that bind to and/or inhibit “activin B”, “activin C”, and “activin E”. Agents disclosed herein that antagonize “activin AB” are agents that inhibit one or more activities as mediated by the β_{A} subunit and one or more activities as mediated by the β_{B} subunit.

The BMPs and GDFs together form a family of cysteine-knot cytokines sharing the characteristic fold of the TGF-beta superfamily [Rider et al. (2010) Biochem J., 429(1):1-12]. This family includes, for example, BMP2, BMP4, BMP6, BMP7, BMP2a, BMP3, BMP3b (also known as GDF10), BMP4, BMP5, BMP6, BMP7, BMP8, BMP8a, BMP8b, BMP9 (also known as GDF2), BMP10, BMP11 (also known as GDF11), BMP12 (also known as GDF7), BMP13 (also known as GDF6), BMP14 (also known as GDF5), BMP15, GDF1, GDF3 (also known as VGR2), GDF8 (also known as myostatin), GDF9, GDF15, and decapentaplegic. Besides the ability to induce bone formation, which gave the BMPs their name, the BMP/GDFs display morphogenetic activities in the development of a wide range of tissues. BMP/GDF homo- and hetero-dimers interact with combinations of type I and type II receptor dimers to produce multiple possible signaling complexes, leading to the activation of one of two competing sets of SMAD transcription factors. BMP/GDFs have highly specific and localized functions. These are regulated in a number of ways, including the developmental restriction of BMP/GDF expression and through the secretion of several specific BMP antagonist proteins that bind with high affinity to the cytokines. Curiously, a number of these antagonists resemble TGF-beta superfamily ligands.

Growth and differentiation factor-8 (GDF8) is also known as myostatin. GDF8 is a negative regulator of skeletal muscle mass and is highly expressed in developing and adult skeletal muscle. The GDF8 null mutation in transgenic mice is characterized by a marked hypertrophy and hyperplasia of skeletal muscle [McPherron et al. Nature (1997) 387:83-90]. Similar increases in skeletal muscle mass are evident in naturally occurring mutations of GDF8 in cattle and, strikingly, in humans [Ashmore et al. (1974) Growth, 38:501-507; Swatland and Kieffer, J. Anim. Sci. (1994) 38:752-757; McPherron and Lee, Proc. Natl. Acad. Sci. USA (1997) 94:12457-12461; Kambadur et al. Genome Res. (1997) 7:910-915; and Schuelke et al. (2004) N Engl J Med, 350:2682-8]. Studies have also shown that muscle wasting associated with HIV-infection in humans is accompanied by increases in GDF8 protein expression [Gonzalez-Cadavid et al., PNAS (1998) 95:14938-43]. In addition, GDF8 can modulate the production of muscle-specific enzymes (e.g., creatine kinase) and modulate myoblast cell proliferation [International Patent Application Publication No. WO 00/43781]. The GDF8 propeptide can noncovalently bind to the mature GDF8 domain dimer, inactivating its biological activity [Miyazono et al. (1988) J. Biol. Chem., 263: 6407-6415; Wakefield et al. (1988) J. Biol. Chem., 263; 7646-7654; and Brown et al. (1990) Growth Factors, 3: 35-43]. Other proteins which bind to GDF8 or structurally related proteins and inhibit their biological activity include follistatin, and potentially, follistatin-related proteins [Gamer et al. (1999) Dev. Biol., 208: 222-232].

GDF11, also known as BMP11, is a secreted protein that is expressed in the tail bud, limb bud, maxillary and mandibular arches, and dorsal root ganglia during mouse development [McPherron et al. (1999) Nat. Genet., 22: 260-264; and Nakashima et al. (1999) Mech. Dev., 80: 185-189]. GDF11 plays a unique role in patterning both mesodermal and neural tissues [Gamer et al. (1999) Dev Biol., 208:222-32]. GDF11 was shown to be a negative regulator of chondrogenesis and myogenesis in developing chick limb [Gamer et al. (2001) Dev Biol., 229:407-20]. The expression of GDF11 in muscle also suggests its role in regulating muscle growth in a similar way to GDF8. In addition, the expression of GDF11 in brain suggests that GDF11 may also possess activities that relate to the function of the nervous system. Interestingly, GDF11 was found to inhibit neurogenesis in the olfactory epithelium [Wu et al. (2003) Neuron., 37:197-207]. Hence, GDF11 may have in vitro and in vivo applications in the treatment of diseases such as muscle diseases and neurodegenerative diseases (e.g., amyotrophic lateral sclerosis).

As used herein ActRII refers to the family of type II activin receptors. This family includes both the activin receptor type IIA (ActRIIA), encoded by the ACVR2A gene, and the activin receptor type IIB (ActRIIB), encoded by the ACVR2B gene. ActRII receptors are TGF-beta superfamily type II receptors that bind a variety of TGF-beta superfamily ligands including activins, GDF8 (myostatin), GDF11, and a subset of BMPs, notably BMP6 and BMP7. ActRII receptors are implicated in a variety of biological disorders including muscle and neuromuscular disorders (e.g., muscular dystrophy, amyotrophic lateral sclerosis (ALS), and muscle atrophy), undesired bone/cartilage growth, adipose tissue disorders (e.g., obesity), metabolic disorders (e.g., type 2 diabetes), and neurodegenerative disorders. See, e.g., Tsuchida et al., (2008) Endocrine Journal 55(1):11-21, Knopf et al., U.S.8,252,900, and OMIM entries 102581 and 602730.

In certain aspects, the present disclosure relates to the use of single-arm ActRIIA heteromultimers or single-arm ActRIIB heteromultimers comprising an extracellular domain of ActRIIA or ActRIIB, respectively, preferably soluble heteromultimers, to antagonize intracellular signaling transduction (e.g., Smad signaling) initiated by one or more ActRIIA or ActRIIB ligands (e.g., activin A, activin B, GDF11, GDF8, GDF3, BMP5, BMP6 and BMP10). As described herein, single-arm ActRIIA heteromultimers or single-arm ActRIIB heteromultimers may be useful for treating a pulmonary disease or condition (e.g., pulmonary hypertension (PH), pulmonary arterial hypertension (PAH), idiopathic pulmonary fibrosis (IPF), interstitial lung disease (ILD)).

As demonstrated herein, a single-arm ActRIIB heterodimer Fc fusion is effective in decreasing blood pressure and cardiac hypertrophy in a PAH model. The rat model for PAH that was used in the studies described herein is considered to be predicative of efficacy in humans, and therefore, this disclosure provides methods for using single-arm ActRIIB heteromultimers, and related antagonists including single-arm ActRIIA heteromultimers, to treat pulmonary hypertension (e.g., PAH), particularly treating, preventing, or reducing the severity or duration of one or more complications of pulmonary hypertension, in humans. While not wishing to be bound to any particular mechanism, it is expected that the effects of these agents is caused primarily by an ActRII signaling antagonist effect. Regardless of the mechanism, it is apparent from the data presented herein that ActRII signaling antagonists decrease blood pressure, decrease cardiac hypertrophy, and have other positivity effects in treating pulmonary hypertension. It should be noted that blood pressure and hypertrophy are dynamic, with changes depending on a balance of factors that increase blood pressure and hypertrophy and factors that decrease blood pressure and hypertrophy. Blood pressure and cardiac hypertrophy can be decreased by increasing factors that reduce blood pressure and cardiac hypertrophy, decreasing factors that promote elevated blood pressure and cardiac hypertrophy, or both. The terms decreasing blood pressure or decreasing cardiac hypertrophy refer to the observable physical changes in blood pressure and cardiac tissue and are intended to be neutral as to the mechanism by which the changes occur.

The terms used in this specification generally have their ordinary meanings in the art, within the context of this disclosure and in the specific context where each term is used. Certain terms are discussed below or elsewhere in the specification to provide additional guidance to the practitioner in describing the compositions and methods of the disclosure and how to make and use them. The scope or meaning of any use of a term will be apparent from the specific context in which it is used.

“Homologous,” in all its grammatical forms and spelling variations, refers to the relationship between two proteins that possess a “common evolutionary origin,” including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions. However, in common usage and in the instant application, the term “homologous,” when modified with an adverb such as “highly,” may refer to sequence similarity and may or may not relate to a common evolutionary origin.

The term “sequence similarity,” in all its grammatical forms, refers to the degree of identity or correspondence between nucleic acid or amino acid sequences that may or may not share a common evolutionary origin.

“Percent (%) sequence identity” with respect to a reference polypeptide (or nucleotide) sequence is defined as the percentage of amino acid residues (or nucleic acids) in a candidate sequence that are identical to the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid (nucleic acid) sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

“Does not substantially bind to X”, in all its grammatical forms, is intended to mean that an agent has a K_{D} that is greater than about 10⁻⁷, 10⁻⁶, 10⁻⁵, 10⁻⁴ or greater (e.g., no detectable binding by the assay used to determine the K_{D}) for “X”.

“Agonize”, in all its grammatical forms, refers to the process of activating a protein and/or gene (e.g., by activating or amplifying that protein’s gene expression or by inducing an inactive protein to enter an active state) or increasing a protein’s and/or gene’s activity.

“Antagonize”, in all its grammatical forms, refers to the process of inhibiting a protein and/or gene (e.g., by inhibiting or decreasing that protein’s gene expression or by inducing an active protein to enter an inactive state) or decreasing a protein’s and/or gene’s activity.

The terms “about” and “approximately” as used in connection with a numerical value throughout the specification and the claims denotes an interval of accuracy, familiar and acceptable to a person skilled in the art. In general, such interval of accuracy is ± 10%. Alternatively, and particularly in biological systems, the terms “about” and “approximately” may mean values that are within an order of magnitude, preferably ≤5-fold and more preferably ≤2-fold of a given value.

Numeric ranges disclosed herein are inclusive of the numbers defining the ranges.

The terms “a” and “an” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Throughout this specification, the word “comprise” or variations such as “comprises” or “comprising” will be understood to imply the inclusion of a stated integer or groups of integers but not the exclusion of any other integer or group of integers.

### 2. Single-Arm Heteromultimers Comprising ActRIIA or ActRIIB Polypeptides

In certain aspects, the disclosure concerns single-arm ActRIIA heteromultimers or single-arm ActRIIB heteromultimers comprising an ActRIIA or ActRIIB polypeptide, respectively. In certain embodiments, the polypeptides disclosed herein may form protein complexes (e.g., heteromultimers) comprising a first polypeptide covalently or non-covalently associated with a second polypeptide, wherein the first polypeptide comprises the amino acid sequence of an ActRIIA or an ActRIIB polypeptide and the amino acid sequence of a first member of an interaction pair; and the second polypeptide comprises the amino acid sequence of a second member of the interaction pair, and wherein the second polypeptide does not comprise an ActRIIA or ActRIIB polypeptide. The interaction pair may be any two polypeptide sequences that interact to form a complex, particularly a heterodimeric complex although operative embodiments may also employ an interaction pair that forms a homodimeric sequence. As described herein, one member of the interaction pair may be fused to an ActRIIA or ActRIIB polypeptide, such as a polypeptide comprising an amino acid sequence that is at least 70%, 75% 80%, 85%, 90%, 91%, 92%, 93%, 94%, 95%, 96%, 97%, 98%, 99% or 100% identical to the sequence of any of SEQ ID NOs: 1, 2, 3, 4, 5, 6, 9, 10, 11, 46, 48, 55, 57, 58, 59, 60, and 61. Preferably, the interaction pair is selected in part to confer an improved serum half-life, or to act as an adapter on to which another moiety, such as a polyethylene glycol moiety, is attached to provide an improved serum half-life relative to the monomeric form of the ActRIIA or ActRIIB polypeptide.

As shown herein, monomeric (single-arm) forms of ActRIIA or ActRIIB can exhibit substantially altered ligand-binding selectivity compared to their corresponding homodimeric forms, but the monomeric forms tend to have a short serum residence time (half-life), which is undesirable in the therapeutic setting. A common mechanism for improving serum half-life is to express a polypeptide as a homodimeric fusion protein with a constant domain portion (e.g., an Fc portion) of an IgG. However, ActRIIA or ActRIIB polypeptides expressed as homodimeric proteins (e.g., in an Fc fusion construct) may not exhibit the same activity profile as the monomeric form. As demonstrated herein, the problem may be solved by fusing the monomeric form to a half-life extending moiety, and surprisingly, this can be readily achieved by expressing such proteins as an asymmetric heterodimeric fusion protein in which one member of an interaction pair is fused to an ActRIIA or ActRIIB polypeptide and another member of the interaction pair is fused to either no moiety or to a heterologous moiety, resulting in a novel ligand-binding profile coupled with an improvement in serum half-life conferred by the interaction pair.

In certain aspects, the present disclosure relates to single-arm heteromultimers comprising an ActRIIA or ActRIIB polypeptide (e.g., a polypeptide comprising the amino acid sequence of any of SEQ ID NOs: 1, 2, 3, 4, 5, 6, 9, 10, 11, 46, 48, 55, 57, 58, 59, 60, and 61), which are generally referred to herein as “single-arm heteromultimers of the disclosure” or “single-arm ActRIIA heteromultimers” or “single-arm ActRIIB heteromultimers”. Preferably, single-arm heteromultimers of the disclosure are soluble, e.g., a single-arm heteromultimer comprises a soluble portion of at least one ActRIIA or ActRIIB polypeptide. In general, the extracellular domains of ActRIIA or ActRIIB correspond to a soluble portion of the ActRIIA or ActRIIB polypeptide. Therefore, in some embodiments, single-arm heteromultimers of the disclosure comprise an extracellular domain of an ActRIIA or ActRIIB polypeptide. Exemplary extracellular domains of ActRIIA and ActRIIB are disclosed herein and such sequences, as well as fragments, functional variants, and modified forms thereof, may be used in accordance with the inventions of the present disclosure (e.g., single-arm heteromultimer compositions and uses thereof).

A defining structural motif known as a three-finger toxin fold is important for ligand binding by ActRIIA or ActRIIB and is formed by 10, 12, or 14 conserved cysteine residues located at varying positions within the extracellular domain of each monomeric receptor. See, e.g., Greenwald et al. (1999) Nat Struct Biol 6:18-22; Hinck (2012) FEBS Lett 586:1860-1870. Any of the heteromeric complexes described herein may comprise such domain of ActRIIA or ActRIIB. The core ligand-binding domains of ActRIIA or ActRIIB, as demarcated by the outermost of these conserved cysteines, correspond to positions 29-109 of SEQ ID NO: 1 (ActRIIB precursor) and positions 30-110 of SEQ ID NO: 9 (ActRIIA precursor). The structurally less-ordered amino acids flanking these cysteine-demarcated core sequences can be truncated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37 residues on either terminus without necessarily altering ligand binding. Exemplary extracellular domains for N-terminal and/or C-terminal truncation include SEQ ID NOs: 2, 3, 5, 6, 10, 11, and 83.

In other preferred embodiments, single-arm heteromultimers of the disclosure bind to and inhibit (antagonize) activity of one or more ActRIIA or ActRIIB ligands including, but not limited to, activin A, activin B, GDF11, GDF8, GDF3, BMP5, BMP6, and BMP10. In particular, single-arm heteromultimers of the disclosure may be used to antagonize intracellular signaling transduction (e.g., Smad signaling) initiated by one or more TGFβ superfamily ligands (e.g., ActRIIA or ActRIIB ligands). As described herein, such antagonist heteromultimers may be for the treatment or prevention of various TGF-beta associated conditions, including without limitation pulmonary diseases or conditions (e.g., pulmonary hypertension (PH), pulmonary arterial hypertension (PAH), idiopathic pulmonary fibrosis (IPF), and interstitial lung disease (ILD)) that are affected by one or more ligands of the TGF-beta superfamily (e.g., ligands of ActRIIA or ActRIIB). In some embodiments, single-arm heteromultimers of the disclosure have different ligand-binding profiles in comparison to their corresponding homomultimer (e.g., a single-arm ActRIIB heterodimer Fc fusion vs. a corresponding single-arm ActRIIB homodimer Fc fusion). As described herein, single-arm heteromultimers of the disclosure include, e.g., heterodimers, heterotrimers, heterotetramers and further oligomeric structures based on a single-arm unitary complex. In certain preferred embodiments, single-arm heteromultimers of the disclosure are heterodimers.

As used herein, the term “ActRIIB” refers to a family of activin receptor type IIB (ActRIIB) proteins from any species and variants derived from such ActRIIB proteins by mutagenesis or other modification. Reference to ActRIIB herein is understood to be a reference to any one of the currently identified forms. Members of the ActRIIB family are generally transmembrane proteins, composed of a ligand-binding extracellular domain comprising a cysteine-rich region, a transmembrane domain, and a cytoplasmic domain with predicted serine/threonine kinase activity.

The term “ActRIIB polypeptide” includes polypeptides comprising any naturally occurring polypeptide of an ActRIIB family member as well as any variants thereof (including mutants, fragments, fusions, and peptidomimetic forms) that retain a useful activity. Examples of such variant ActRIIB polypeptides are provided throughout the present disclosure as well as in International Patent Application Publication Nos. WO 2006/012627 and WO 2008/097541, which are incorporated herein by reference in its entirety. Numbering of amino acids for all ActRIIB-related polypeptides described herein is based on the numbering of the human ActRIIB precursor protein sequence provided below (SEQ ID NO: 1), unless specifically designated otherwise.

The human ActRIIB precursor protein sequence is as follows:

1 MTAPWVALAL LWGSLCAGSG RGEAETRECI YYNANWELER TNQSGLERCE
51 GEQDKRLHCY ASWRNSSGTI ELVKKGCWLD DFNCYDRQEC VATEENPQVY
101 FCCCEGNFCN ERFTHLPEAG GPEVTYEPPP TAPTLLTVLA YSLLPIGGLS
151 LIVLLAFWMY RHRKPPYGHV DIHEDPGPPP PSPLVGLKPL QLLEIKARGR
201 FGCVWKAQLM NDFVAVKIFP LQDKQSWQSE REIFSTPGMK HENLLQFIAA
251 EKRGSNLEVE LWLITAFHDK GSLTDYLKGN IITWNELCHV AETMSRGLSY
301 LHEDVPWCRG EGHKPSIAHR DFKSKNVLLK SDLTAVLADF GLAVRFEPGK
351 PPGDTHGQVG TRRYMAPEVL EGAINFQRDA FLRIDMYAMG LVLWELVSRC
401 KAADGPVDEY MLPFEEEIGQ HPSLEELQEV VVHKKMRPTI KDHWLKHPGL
451 AQLCVTIEEC WDHDAEARLS AGCVEERVSL IRRSVNGTTS DCLVSLVTSV
501 TNVDLPPKES SI (SEQ ID NO: 1)

The signal peptide is indicated with a single underline; the extracellular domain is indicated in bold font; and the potential, endogenous N-linked glycosylation sites are indicated with a double underline.

A processed extracellular ActRIIB polypeptide sequence is as follows:

GRGEAETRECIYYNANWELERTNQSGLERCEGEQDKRLHCYASWRNSSGTIELVKKGCWLDD
FNCYDRQECVATEENPQVYFCCCEGNFCNERFTHLPEAGGPEVTYEPPPTAPT (SEQ ID
NO: 2).

In some embodiments, the protein may be produced with an “SGR... ” sequence at the N-terminus. The C-terminal “tail” of the extracellular domain is indicated by a single underline. The sequence with the “tail” deleted (a Δ15 sequence) is as follows:

GRGEAETRECIYYNANWELERTNQSGLERCEGEQDKRLHCYASWRNSSGTIELVKKGCWLDD
FNCYDRQECVATEENPQVYFCCCEGNFCNERFTHLPEA (SEQ ID NO: 3).

A form of ActRIIB with an alanine at position 64 of SEQ ID NO: 1 (A64) is also reported in the literature See, e.g., Hilden et al. (1994) Blood, 83(8): 2163-2170. Applicants have ascertained that an ActRIIB-Fc fusion protein comprising an extracellular domain of ActRIIB with the A64 substitution has a relatively low affinity for activin and GDF11. By contrast, the same ActRIIB-Fc fusion protein with an arginine at position 64 (R64) has an affinity for activin and GDF11 in the low nanomolar to high picomolar range. Therefore, sequences with an R64 are used as the “wild-type” reference sequence for human ActRIIB in this disclosure.

The form of ActRIIB with an alanine at position 64 is as follows:

1 MTAPWVALAL LWGSLCAGSG RGEAETRECI YYNANWELER TNQSGLERCE
51 GEQDKRLHCY ASWANSSGTI ELVKKGCWLD DFNCYDRQEC VATEENPQVY
101 FCCCEGNFCN ERFTHLPEAG GPEVTYEPPP TAPTLLTVLA YSLLPIGGLS
151 LIVLLAFWMY RHRKPPYGHV DIHEDPGPPP PSPLVGLKPL QLLEIKARGR
201 FGCVWKAQLM NDFVAVKIFP LQDKQSWQSE REIFSTPGMK HENLLQFIAA
251 EKRGSNLEVE LWLITAFHDK GSLTDYLKGN IITWNELCHV AETMSRGLSY
301 LHEDVPWCRG EGHKPSIAHR DFKSKNVLLK SDLTAVLADF GLAVRFEPGK
351 PPGDTHGQVG TRRYMAPEVL EGAINFQRDA FLRIDMYAMG LVLWELVSRC
401 KAADGPVDEY MLPFEEEIGQ HPSLEELQEV VVHKKMRPTI KDHWLKHPGL
451 AQLCVTIEEC WDHDAEARLS AGCVEERVSL IRRSVNGTTS DCLVSLVTSV
501 TNVDLPPKES SI (SEQ ID NO: 4)

The signal peptide is indicated by single underline and the extracellular domain is indicated by bold font.

The processed extracellular ActRIIB polypeptide sequence of the alternative A64 form is as follows:

GRGEAETRECIYYNANWELERTNQSGLERCEGEQDKRLHCYASWANSSGTIELVKKGCWLDD
FNCYDRQECVATEENPQVYFCCCEGNFCNERFTHLPEAGGPEVTYEPPPTAPT (SEQ ID
NO: 5)

In some embodiments, the protein may be produced with an “SGR... ” sequence at the N-terminus. The C-terminal “tail” of the extracellular domain is indicated by single underline. The sequence with the “tail” deleted (a Δ15 sequence) is as follows:

GRGEAETRECIYYNANWELERTNQSGLERCEGEQDKRLHCYASWANSSGTIELVKKGCWLDD
FNCYDRQECVATEENPQVYFCCCEGNFCNERFTHLPEA (SEQ ID NO: 6)

A nucleic acid sequence encoding the human ActRIIB precursor protein is shown below (SEQ ID NO: 7), consisting of nucleotides 25-1560 of Genbank Reference Sequence NM_001106.3, which encode amino acids 1-513 of the ActRIIB precursor. The sequence as shown provides an arginine at position 64 and may be modified to provide an alanine instead. The signal sequence is underlined.

1 ATGACGGCGC CCTGGGTGGC CCTCGCCCTC CTCTGGGGAT CGCTGTGCGC
51 CGGCTCTGGG CGTGGGGAGG CTGAGACACG GGAGTGCATC TACTACAACG
101 CCAACTGGGA GCTGGAGCGC ACCAACCAGA GCGGCCTGGA GCGCTGCGAA
151 GGCGAGCAGG ACAAGCGGCT GCACTGCTAC GCCTCCTGGC GCAACAGCTC
201 TGGCACCATC GAGCTCGTGA AGAAGGGCTG CTGGCTAGAT GACTTCAACT
251 GCTACGATAG GCAGGAGTGT GTGGCCACTG AGGAGAACCC CCAGGTGTAC
301 TTCTGCTGCT GTGAAGGCAA CTTCTGCAAC GAACGCTTCA CTCATTTGCC
351 AGAGGCTGGG GGCCCGGAAG TCACGTACGA GCCACCCCCG ACAGCCCCCA
401 CCCTGCTCAC GGTGCTGGCC TACTCACTGC TGCCCATCGG GGGCCTTTCC
451 CTCATCGTCC TGCTGGCCTT TTGGATGTAC CGGCATCGCA AGCCCCCCTA
501 CGGTCATGTG GACATCCATG AGGACCCTGG GCCTCCACCA CCATCCCCTC
551 TGGTGGGCCT GAAGCCACTG CAGCTGCTGG AGATCAAGGC TCGGGGGCGC
601 TTTGGCTGTG TCTGGAAGGC CCAGCTCATG AATGACTTTG TAGCTGTCAA
651 GATCTTCCCA CTCCAGGACA AGCAGTCGTG GCAGAGTGAA CGGGAGATCT
701 TCAGCACACC TGGCATGAAG CACGAGAACC TGCTACAGTT CATTGCTGCC
751 GAGAAGCGAG GCTCCAACCT CGAAGTAGAG CTGTGGCTCA TCACGGCCTT
801 CCATGACAAG GGCTCCCTCA CGGATTACCT CAAGGGGAAC ATCATCACAT
851 GGAACGAACT GTGTCATGTA GCAGAGACGA TGTCACGAGG CCTCTCATAC
901 CTGCATGAGG ATGTGCCCTG GTGCCGTGGC GAGGGCCACA AGCCGTCTAT
951 TGCCCACAGG GACTTTAAAA GTAAGAATGT ATTGCTGAAG AGCGACCTCA
1001 CAGCCGTGCT GGCTGACTTT GGCTTGGCTG TTCGATTTGA GCCAGGGAAA
1051 CCTCCAGGGG ACACCCACGG ACAGGTAGGC ACGAGACGGT ACATGGCTCC
1101 TGAGGTGCTC GAGGGAGCCA TCAACTTCCA GAGAGATGCC TTCCTGCGCA
1151 TTGACATGTA TGCCATGGGG TTGGTGCTGT GGGAGCTTGT GTCTCGCTGC
1201 AAGGCTGCAG ACGGACCCGT GGATGAGTAC ATGCTGCCCT TTGAGGAAGA
1251 GATTGGCCAG CACCCTTCGT TGGAGGAGCT GCAGGAGGTG GTGGTGCACA
1301 AGAAGATGAG GCCCACCATT AAAGATCACT GGTTGAAACA CCCGGGCCTG
1351 GCCCAGCTTT GTGTGACCAT CGAGGAGTGC TGGGACCATG ATGCAGAGGC
1401 TCGCTTGTCC GCGGGCTGTG TGGAGGAGCG GGTGTCCCTG ATTCGGAGGT
1451 CGGTCAACGG CACTACCTCG GACTGTCTCG TTTCCCTGGT GACCTCTGTC
1501 ACCAATGTGG ACCTGCCCCC TAAAGAGTCA AGCATC (SEQ ID NO: 7)

A nucleic acid sequence encoding processed extracellular human ActRIIB polypeptide is as follows (SEQ ID NO: 8). The sequence as shown provides an arginine at position 64, and may be modified to provide an alanine instead.

1 GGGCGTGGGG AGGCTGAGAC ACGGGAGTGC ATCTACTACA ACGCCAACTG
51 GGAGCTGGAG CGCACCAACC AGAGCGGCCT GGAGCGCTGC GAAGGCGAGC
101 AGGACAAGCG GCTGCACTGC TACGCCTCCT GGCGCAACAG CTCTGGCACC
151 ATCGAGCTCG TGAAGAAGGG CTGCTGGCTA GATGACTTCA ACTGCTACGA
201 TAGGCAGGAG TGTGTGGCCA CTGAGGAGAA CCCCCAGGTG TACTTCTGCT
251 GCTGTGAAGG CAACTTCTGC AACGAACGCT TCACTCATTT GCCAGAGGCT
301 GGGGGCCCGG AAGTCACGTA CGAGCCACCC CCGACAGCCC CCACC
(SEQ ID NO: 8)

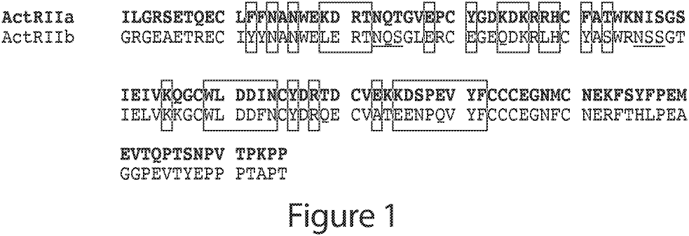

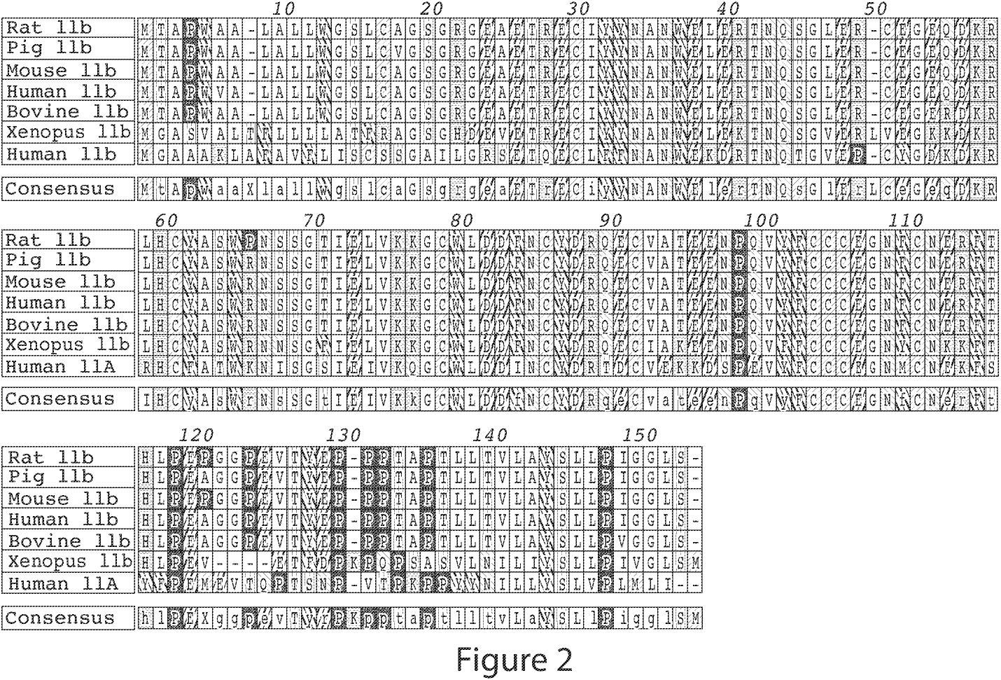

An alignment of the amino acid sequences of human ActRIIB soluble extracellular domain and human ActRIIA soluble extracellular domain are illustrated in FIG. 1. This alignment indicates amino acid residues within both receptors that are believed to directly contact ActRII ligands. FIG. 2 depicts a multiple-sequence alignment of various vertebrate ActRIIB proteins and human ActRIIA. From these alignments is it possible to predict key amino acid positions within the ligand-binding domain that are important for normal ActRII-ligand binding activities as well as to predict amino acid positions that are likely to be tolerant to substitution without significantly altering normal ActRII-ligand binding activities. ActRII proteins have been characterized in the art in terms of structural and functional characteristics, particularly with respect to ligand binding. See, e.g., Attisano et al. (1992) Cell 68(1):97-108; Greenwald et al. (1999) Nature Structural Biology 6(1): 18-22; Allendorph et al. (2006) PNAS 103(20: 7643-7648; Thompson et al. (2003) The EMBO Journal 22(7): 1555-1566; as well as U.S. Pat. Nos: 7,709,605, 7,612,041, and 7,842,663.

For example, Attisano et al. showed that a deletion of the proline knot at the C-terminus of the extracellular domain of ActRIIB reduced the affinity of the receptor for activin. An ActRIIB-Fc fusion protein containing amino acids 20-119 of present SEQ ID NO: 1, “ActRIIB(20-119)-Fc”, has reduced binding to GDF11 and activin relative to an ActRIIB(20-134)-Fc, which includes the proline knot region and the complete juxtamembrane domain (see, e.g., U.S. Pat. No. 7,842,663). However, an ActRIIB(20-129)-Fc protein retains similar but somewhat reduced activity relative to the wild-type, even though the proline knot region is disrupted. Thus, ActRIIB extracellular domains that stop at amino acid 134, 133, 132, 131, 130 and 129 (with respect to SEQ ID NO: 1) are all expected to be active, but constructs stopping at 134 or 133 may be most active. Similarly, mutations at any of residues 129-134 (with respect to SEQ ID NO: 1) are not expected to alter ligand-binding affinity by large margins. In support of this, it is known in the art that mutations of P129 and P130 (with respect to SEQ ID NO: 1) do not substantially decrease ligand binding. Therefore, an ActRIIB polypeptide of the present disclosure may end as early as amino acid 109 (the final cysteine), however, forms ending at or between 109 and 119 (e.g., 109, 110, 111, 112, 113, 114, 115, 116, 117, 118, or 119) are expected to have reduced ligand binding. Amino acid 119 (with respect to present SEQ ID NO:1) is poorly conserved and so is readily altered or truncated. ActRIIB polypeptides and ActRIIB-based GDF traps ending at 128 (with respect to SEQ ID NO: 1) or later should retain ligand-binding activity. ActRIIB polypeptides and ActRIIB-based GDF traps ending at or between 119 and 127 (e.g., 119, 120, 121, 122, 123, 124, 125, 126, or 127),with respect to SEQ ID NO: 1, will have an intermediate binding ability. Any of these forms may be desirable to use, depending on the clinical or experimental setting.

At the N-terminus of ActRIIB, it is expected that a protein beginning at amino acid 29 or before (with respect to SEQ ID NO: 1) will retain ligand-binding activity. Amino acid 29 represents the initial cysteine. An alanine-to-asparagine mutation at position 24 (with respect to SEQ ID NO: 1) introduces an N-linked glycosylation sequence without substantially affecting ligand binding. See, e.g., U.S. Pat. No. 7,842,663. This confirms that mutations in the region between the signal cleavage peptide and the cysteine cross-linked region, corresponding to amino acids 20-29, are well tolerated. In particular, ActRIIB polypeptides and ActRIIB-based GDF traps beginning at position 20, 21, 22, 23, and 24 (with respect to SEQ ID NO: 1) should retain general ligand-biding activity, and ActRIIB polypeptides and ActRIIB-based GDF traps beginning at positions 25, 26, 27, 28, and 29 (with respect to SEQ ID NO: 1) are also expected to retain ligand-biding activity. Data shown in, e.g., U.S. Pat. No. 7,842,663 demonstrates that, surprisingly, an ActRIIB construct beginning at 22, 23, 24, or 25 will have the most activity.

Taken together, an active portion (e.g., ligand-binding portion) of ActRIIB comprises amino acids 29-109 of SEQ ID NO: 1. Therefore ActRIIB polypeptides of the present disclosure may, for example, comprise an amino acid sequence that is at least 80%, 85%, 90%, 91%, 92%, 93%, 94%, 95%, 96%, 97%, 98%, 99%, or 100% identical to a portion of ActRIIB beginning at a residue corresponding to amino acids 20-29 (e.g., beginning at amino acid 20, 21, 22, 23, 24, 25, 26, 27, 28, or 29) of SEQ ID NO: 1 and ending at a position corresponding to amino acids 109-134 (e.g., ending at amino acid 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 1. Other examples include polypeptides that begin at a position from 20-29 (e.g., position 20, 21, 22, 23, 24, 25, 26, 27, 28, or 29) or 21-29 (e.g., position 21, 22, 23, 24, 25, 26, 27, 28, or 29) and end at a position from 119-134 (e.g., 119, 120, 121, 122, 123, 124, 125, 126, 127, 128, 129, 130, 131, 132, 133, or 134), 119-133 (e.g., 119, 120, 121, 122, 123, 124, 125, 126, 127, 128, 129, 130, 131, 132, or 133), 129-134 (e.g., 129, 130, 131, 132, 133, or 134), or 129-133 (e.g., 129, 130, 131, 132, or 133) of SEQ ID NO: 1. Other examples include constructs that begin at a position from 20-24 (e.g., 20, 21, 22, 23, or 24), 21-24 (e.g., 21, 22, 23, or 24), or 22-25 (e.g., 22, 22, 23, or 25) and end at a position from 109-134 (e.g., 109, 110, 111, 112, 113, 114, 115, 116, 117, 118, 119, 120, 121, 122, 123, 124, 125, 126, 127, 128, 129, 130, 131, 132, 133, or 134), 119-134 (e.g., 119, 120, 121, 122, 123, 124, 125, 126, 127, 128, 129, 130, 131, 132, 133, or 134) or 129-134 (e.g., 129, 130, 131, 132, 133, or 134) of SEQ ID NO: 1. Variants within these ranges are also contemplated, particularly those having at least 80%, 85%, 90%, 91%, 92%, 93%, 94%, 95%, 96%, 97%, 98%, 99%, or 100% identity to the corresponding portion of SEQ ID NO: 1.

The disclosure includes the results of an analysis of composite ActRIIB structures, shown in FIG. 2, demonstrating that the ligand-binding pocket is defined, in part, by residues Y31, N33, N35, L38 through T41, E47, E50, Q53 through K55, L57, H58, Y60, S62, K74, W78 through N83, Y85, R87, A92, and E94 through F101. Additionally, ActRIIB is well-conserved across nearly all vertebrates, with large stretches of the extracellular domain conserved completely. Accordingly, comparisons of ActRIIB sequences from various vertebrate organisms provide insights into residues that may be altered. For example, R40 is a K in Xenopus, indicating that basic amino acids at this position will be tolerated. L46 is a valine in Xenopus ActRIIB, and so this position may be altered, and optionally may be altered to another hydrophobic residue, such as V, I or F, or a non-polar residue such as A. E52 is a K in Xenopus, indicating that this site may be tolerant of a wide variety of changes, including polar residues, such as E, D, K, R, H, S, T, P, G, Y and probably A. Q53 is R in bovine ActRIIB and K in Xenopus ActRIIB, and therefore amino acids including R, K, Q, N and H will be tolerated at this position. T93 is a K in Xenopus, indicating that a wide structural variation is tolerated at this position, with polar residues favored, such as S, K, R, E, D, H, G, P, G and Y. F108 is a Y in Xenopus, and therefore Y or other hydrophobic group, such as I, V or L should be tolerated. E111 is K in Xenopus, indicating that charged residues will be tolerated at this position, including D, R, K and H, as well as Q and N. R112 is K in Xenopus, indicating that basic residues are tolerated at this position, including R and H. A at position 119 is relatively poorly conserved, and appears as P in rodents and V in Xenopus, thus essentially any amino acid should be tolerated at this position. The variations described herein may be combined in various ways. Additionally, the results of a mutagenesis program described in the art also confirms that there are amino acid positions in ActRIIB that are often beneficial to conserve. With respect to SEQ ID NO: 1, these include position 64 (basic amino acid), position 80 (acidic or hydrophobic amino acid), position 78 (hydrophobic, and particularly tryptophan), position 37 (acidic, and particularly aspartic or glutamic acid), position 56 (basic amino acid), position 60 (hydrophobic amino acid, particularly phenylalanine or tyrosine). Thus, in the ActRIIB polypeptides disclosed herein, the disclosure provides a framework of amino acids that may be conserved. Other positions that may be desirable to conserve are as follows: position 52 (acidic amino acid), position 55 (basic amino acid), position 81 (acidic), 98 (polar or charged, particularly E, D, R or K), all with respect to SEQ ID NO: 1.

Thus, a general formula for an ActRIIB polypeptide of the disclosure is one that comprises an amino acid sequence that is at least 80%, 85%, 90%, 91%, 92%, 93%, 94%, 95%, 96%, 97%, 98%, 99%, or 100% identical to amino acids 29-109 of SEQ ID NO: 1, optionally beginning at a position ranging from 20-24 (e.g., 20, 21, 22, 23, or 24) or 22-25(e.g., 22, 23, 24, or 25) and ending at a position ranging from 129-134 (e.g., 129, 130, 131, 132, 133, or 134), and comprising no more than 1, 2, 5, 10 or 15 conservative amino acid changes in the ligand-binding pocket, and zero, one or more non-conservative alterations at positions 40, 53, 55, 74, 79 and/or 82 in the ligand-binding pocket. Sites outside the binding pocket, at which variability may be particularly well tolerated, include the amino and carboxy termini of the extracellular domain (as noted above), and positions 42-46 and 65-73 (with respect to SEQ ID NO: 1). An asparagine-to-alanine alteration at position 65 (N65A) actually improves ligand binding in the A64 background, and is thus expected to have no detrimental effect on ligand binding in the R64 background. See, e.g., U.S. Pat. No. 7,842,663. This change probably eliminates glycosylation at N65 in the A64 background, thus demonstrating that a significant change in this region is likely to be tolerated. While an R64A change is poorly tolerated, R64K is well-tolerated, and thus another basic residue, such as H may be tolerated at position 64. See, e.g., U.S. Pat. No. 7,842,663.

In certain embodiments, the disclosure relates to single-arm heteromultimers that comprise at least one ActRIIB polypeptide, which includes fragments, functional variants, and modified forms thereof. Preferably, ActRIIB polypeptides for use in accordance with inventions of the disclosure (e.g., single-arm heteromultimers comprising an ActRIIB polypeptide and uses thereof) are soluble (e.g., an extracellular domain of ActRIIB). In other preferred embodiments, ActRIIB polypeptides for use in accordance with the inventions of the disclosure bind to and/or inhibit (antagonize) activity (e.g., induction Smad signaling) of one or more TGF-beta superfamily ligands. In some embodiments, single-arm heteromultimers of the disclosure comprise at least one ActRIIB polypeptide that comprises, consists, or consists essentially of an amino acid sequence that is at least 80%, 85%, 90%, 91%, 92%, 93%, 94%, 95%, 96%, 97%, 98%, 99%, or 100% identical to a portion of ActRIIB beginning at a residue corresponding to amino acids 20-29 (e.g., beginning at amino acid 20, 21, 22, 23, 24, 25, 26, 27, 28, or 29) of SEQ ID NO: 1 and ending at a position corresponding to amino acids 109-134 (e.g., ending at amino acid 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 1. In some embodiments, single-arm heteromultimers of the disclosure comprise at least one ActRIIB polypeptide that comprises, consists, or consists essentially of an amino acid sequence that is at least 80%, 85%, 90%, 91%, 92%, 93%, 94%, 95%, 96%, 97%, 98%, 99%, or 100% identical to a portion of ActRIIB beginning at a residue corresponding to amino acids 20-29 (e.g., beginning at amino acid 20, 21, 22, 23, 24, 25, 26, 27, 28, or 29) of SEQ ID NO: 1 and ending at a position corresponding to amino acids 109-134 (e.g., ending at amino acid 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 1, wherein the position corresponding to L79 of SEQ ID NO: 1 is an acidic amino acid (i.e., a D or E amino acid residue). In certain preferred embodiments, single-arm heteromultimers of the disclosure comprise at least one ActRIIB polypeptide that comprises, consists, or consists essentially of an amino acid sequence that is at least 80%, 85%, 90%, 91%, 92%, 93%, 94%, 95%, 96%, 97%, 98%, 99%, or 100% identical amino acids 29-109 of SEQ ID NO: 1. In other preferred embodiments, single-arm heteromultimers of the disclosure comprise at least one ActRIIB polypeptide that comprises, consists, or consists essentially of an amino acid sequence that is at least 80%, 85%, 90%, 91%, 92%, 93%, 94%, 95%, 96%, 97%, 98%, 99%, or 100% identical amino acids 29-109 of SEQ ID NO: 1, wherein the position corresponding to L79 of SEQ ID NO: 1 is an acidic amino acid (i.e., a D or E amino acid residue). In other preferred embodiments, single-arm heteromultimers of the disclosure comprise at least one ActRIIB polypeptide that comprises, consists, or consists essentially of an amino acid sequence that is at least 80%, 85%, 90%, 91%, 92%, 93%, 94%, 95%, 96%, 97%, 98%, 99%, or 100% identical amino acids 29-109 of SEQ ID NO: 1, wherein the position corresponding to L79 of SEQ ID NO: 1 is not an acidic amino acid (i.e., a D or E amino acid residue).In some embodiments, single-arm heteromultimers of the disclosure comprise at least one ActRIIB polypeptide that is at least 70%, 75%, 80%, 85%, 90%, 91%, 92%, 93%, 94%, 95%, 96%, 97%, 98%, or 99% identical to the amino acid sequence of any one of SEQ ID NOs: 1, 2, 3, 4, 5, 6, 46, 48, 60, or 61. In some embodiments, single-arm heteromultimers of the disclosure comprise at least one ActRIIB polypeptide that is at least 70%, 75%, 80%, 85%, 90%, 91%, 92%, 93%, 94%, 95%, 96%, 97%, 98%, 99%, or 100%identical to the amino acid sequence of any one of SEQ ID NOs: 1, 2, 3, 4, 5, 6, 46, 48, 60, or 61, wherein the position corresponding to L79 of SEQ ID NO: 1 is an acidic amino acid (i.e., a D or E amino acid residue). In some embodiments, single-arm heteromultimers of the disclosure comprise at least one ActRIIB polypeptide that is at least 70%, 75%, 80%, 85%, 90%, 91%, 92%, 93%, 94%, 95%, 96%, 97%, 98%, 99%, or 100% identical to the amino acid sequence of any one of SEQ ID NOs: 1, 2, 3, 4, 5, 6, 46, 48, 60, or 61, wherein the position corresponding to L79 of SEQ ID NO: 1 is not an acidic amino acid (i.e., a D or E amino acid residue). In some embodiments, single-arm heteromultimers of the disclosure comprise, consist, or consist essentially of at least one ActRIIB polypeptide that is at least 70%, 75%, 80%, 85%, 90%, 91%, 92%, 93%, 94%, 95%, 96%, 97%, 98%, 99%, or 100%identical to the amino acid sequence of any one of SEQ ID NOs: 1, 2, 3, 4, 5, 6, 46, 48, 60, or 61. In some embodiments, single-arm heteromultimers of the disclosure comprise, consist, or consist essentially of at least one ActRIIB polypeptide that is at least 70%, 75%, 80%, 85%, 90%, 91%, 92%, 93%, 94%, 95%, 96%, 97%, 98%, 99%, or 100%identical to the amino acid sequence of any one of SEQ ID NOs: 1, 2, 3, 4, 5, 6, 46, 48, 60, or 61, wherein the position corresponding to L79 of SEQ ID NO: 1 is an acidic amino acid (i.e., a D or E amino acid residue). In some embodiments, single-arm heteromultimers of the disclosure comprise, consist, or consist essentially of at least one ActRIIB polypeptide that is at least 70%, 75%, 80%, 85%, 90%, 91%, 92%, 93%, 94%, 95%, 96%, 97%, 98%, 99%, or 100% identical to the amino acid sequence of any one of SEQ ID NOs: 1, 2, 3, 4, 5, 6, 46, 48, 60, or 61, wherein the position corresponding to L79 of SEQ ID NO: 1 is not an acidic amino acid (i.e., a D or E amino acid residue).

In some embodiments, single-arm heteromultimers of the disclosure comprise, consist, or consist essentially of at least one ActRIIB polypeptide that is at least 70%, 75%, 80%, 85%, 90%, 91%, 92%, 93%, 94%, 95%, 96%, 97%, 98%, 99%, or 100% identical to the amino acid sequence of SEQ ID NO: 83, wherein the position corresponding to L79 is an aspartic acid (D). The amino acid sequence for the truncated GDF trap ActRIIB(L79D 25-131) without the leader, hFc domain, and linker (SEQ ID NO: 83) is shown below. The aspartate substituted at position 79 in the native sequence is underlined and bolded, as is the glutamate revealed by sequencing to be the N-terminal residue in the mature fusion protein.

1 E TRECIYYNA NWELERTNQS GLERCEGEQD KRLHCYASWR
NSSGTIELVK
51 KGCWD DDFNC YDRQECVATE ENPQVYFCCC EGNFCNERFT
HLPEAGGPEV
111 TYEPPPT (SEQ ID NO: 83)

In certain embodiments, the present disclosure relates to a protein complex comprising an ActRIIA polypeptide. As used herein, the term “ActRIIA” refers to a family of activin receptor type IIA (ActRIIA) proteins from any species and variants derived from such ActRIIA proteins by mutagenesis or other modification. Reference to ActRIIA herein is understood to be a reference to any one of the currently identified forms. Members of the ActRIIA family are generally transmembrane proteins, composed of a ligand-binding extracellular domain comprising a cysteine-rich region, a transmembrane domain, and a cytoplasmic domain with predicted serine/threonine kinase activity.

The term “ActRIIA polypeptide” includes polypeptides comprising any naturally occurring polypeptide of an ActRIIA family member as well as any variants thereof (including mutants, fragments, fusions, and peptidomimetic forms) that retain a useful activity. Examples of such variant ActRIIA polypeptides are provided throughout the present disclosure as well as in International Pat. Application Publication No. WO 2006/012627, which is incorporated herein by reference in its entirety. Numbering of amino acids for all ActRIIA-related polypeptides described herein is based on the numbering of the human ActRIIA precursor protein sequence provided below (SEQ ID NO: 9), unless specifically designated otherwise.

The human ActRIIA precursor protein sequence is as follows:

1 MGAAAKLAFA VELISCSSGA ILGRSETQEC LFFNANWEKD RTNQTGVEPC
51 YGDKDKRRHC FATWKNISGS IEIVKQGCWL DDINCYDRTD CVEKKDSPEV
101 YFCCCEGNMC NEKFSYFPEM EVTQPTSNPV TPKPPYYNIL LYSLVPLMLI
151 AGIVICAFWV YRHHKMAYPP VLVPTQDPGP PPPSPLLGLK PLQLLEVKAR
201 GRFGCVWKAQ LLNEYVAVKI FPIQDKQSWQ NEYEVYSLPG MKHENILQFI
251 GAEKRGTSVD VDLWLITAFH EKGSLSDFLK ANWSWNELC HIAETMARGL
301 AYLHEDIPGL KDGHKPAISH RDIKSKNVLL KNNLTACIAD FGLALKFEAG
351 KSAGDTHGQV GTRRYMAPEV LEGAINFQRD AFLRIDMYAM GLVLWELASR
401 CTAADGPVDE YMLPFEEEIG QHPSLEDMQE VVVHKKKRPV LRDYWQKHAG
451 MAMLCETIEE CWDHDAEARL SAGCVGERIT QMQRLTNIIT TEDIVTVVTM
501 VTNVDFPPKE SSL (SEQ ID NO: 9)

The signal peptide is indicated by a single underline; the extracellular domain is indicated in bold font; and the potential, endogenous N-linked glycosylation sites are indicated by a double underline.

The processed extracellular human ActRIIA polypeptide sequence is as follows:

ILGRSETQECLFFNANWEKDRTNQTGVEPCYGDKDKRRHCFATWKNISGSIEIVKQGCWLDD
INCYDRTDCVEKKDSPEVYFCCCEGNMCNEKFSYFPEMEVTQPTSNPVTPKPP (SEQ ID
NO: 10)

The C-terminal “tail” of the extracellular domain is indicated by a single underline. The sequence with the “tail” deleted (a Δ15 sequence) is as follows:

ILGRSETQECLFFNANWEKDRTNQTGVEPCYGDKDKRRHCFATWKNISGSIEIVKQGCWLDD
INCYDRTDCVEKKDSPEVYFCCCEGNMCNEKFSYFPEM (SEQ ID NO: 11)

A nucleic acid sequence encoding the human ActRIIA precursor protein is shown below (SEQ ID NO: 12), corresponding to nucleotides 159-1700 of Genbank Reference Sequence NM_001616.4. The signal sequence is underlined.

1 ATGGGAGCTG CTGCAAAGTT GGCGTTTGCC GTCTTTCTTA TCTCCTGTTC
51 TTCAGGTGCT ATACTTGGTA GATCAGAAAC TCAGGAGTGT CTTTTCTTTA
101 ATGCTAATTG GGAAAAAGAC AGAACCAATC AAACTGGTGT TGAACCGTGT
151 TATGGTGACA AAGATAAACG GCGGCATTGT TTTGCTACCT GGAAGAATAT
201 TTCTGGTTCC ATTGAAATAG TGAAACAAGG TTGTTGGCTG GATGATATCA
251 ACTGCTATGA CAGGACTGAT TGTGTAGAAA AAAAAGACAG CCCTGAAGTA
301 TATTTTTGTT GCTGTGAGGG CAATATGTGT AATGAAAAGT TTTCTTATTT
351 TCCGGAGATG GAAGTCACAC AGCCCACTTC AAATCCAGTT ACACCTAAGC
401 CACCCTATTA CAACATCCTG CTCTATTCCT TGGTGCCACT TATGTTAATT
451 GCGGGGATTG TCATTTGTGC ATTTTGGGTG TACAGGCATC ACAAGATGGC
501 CTACCCTCCT GTACTTGTTC CAACTCAAGA CCCAGGACCA CCCCCACCTT
551 CTCCATTACT AGGTTTGAAA CCACTGCAGT TATTAGAAGT GAAAGCAAGG
601 GGAAGATTTG GTTGTGTCTG GAAAGCCCAG TTGCTTAACG AATATGTGGC
651 TGTCAAAATA TTTCCAATAC AGGACAAACA GTCATGGCAA AATGAATACG
701 AAGTCTACAG TTTGCCTGGA ATGAAGCATG AGAACATATT ACAGTTCATT
751 GGTGCAGAAA AACGAGGCAC CAGTGTTGAT GTGGATCTTT GGCTGATCAC
801 AGCATTTCAT GAAAAGGGTT CACTATCAGA CTTTCTTAAG GCTAATGTGG
851 TCTCTTGGAA TGAACTGTGT CATATTGCAG AAACCATGGC TAGAGGATTG
901 GCATATTTAC ATGAGGATAT ACCTGGCCTA AAAGATGGCC ACAAACCTGC
951 CATATCTCAC AGGGACATCA AAAGTAAAAA TGTGCTGTTG AAAAACAACC
1001 TGACAGCTTG CATTGCTGAC TTTGGGTTGG CCTTAAAATT TGAGGCTGGC
1051 AAGTCTGCAG GCGATACCCA TGGACAGGTT GGTACCCGGA GGTACATGGC
1101 TCCAGAGGTA TTAGAGGGTG CTATAAACTT CCAAAGGGAT GCATTTTTGA
1151 GGATAGATAT GTATGCCATG GGATTAGTCC TATGGGAACT GGCTTCTCGC
1201 TGTACTGCTG CAGATGGACC TGTAGATGAA TACATGTTGC CATTTGAGGA
1251 GGAAATTGGC CAGCATCCAT CTCTTGAAGA CATGCAGGAA GTTGTTGTGC
1301 ATAAAAAAAA GAGGCCTGTT TTAAGAGATT ATTGGCAGAA ACATGCTGGA
1351 ATGGCAATGC TCTGTGAAAC CATTGAAGAA TGTTGGGATC ACGACGCAGA
1401 AGCCAGGTTA TCAGCTGGAT GTGTAGGTGA AAGAATTACC CAGATGCAGA
1451 GACTAACAAA TATTATTACC ACAGAGGACA TTGTAACAGT GGTCACAATG
1501 GTGACAAATG TTGACTTTCC TCCCAAAGAA TCTAGTCTA
(SEQ ID NO: 12)

The nucleic acid sequence encoding processed extracellular ActRIIA polypeptide is as follows:

1 ATACTTGGTA GATCAGAAAC TCAGGAGTGT CTTTTCTTTA ATGCTAATTG
51 GGAAAAAGAC AGAACCAATC AAACTGGTGT TGAACCGTGT TATGGTGACA
101 AAGATAAACG GCGGCATTGT TTTGCTACCT GGAAGAATAT TTCTGGTTCC
151 ATTGAAATAG TGAAACAAGG TTGTTGGCTG GATGATATCA ACTGCTATGA
201 CAGGACTGAT TGTGTAGAAA AAAAAGACAG CCCTGAAGTA TATTTTTGTT
251 GCTGTGAGGG CAATATGTGT AATGAAAAGT TTTCTTATTT TCCGGAGATG
301 GAAGTCACAC AGCCCACTTC AAATCCAGTT ACACCTAAGC CACCC
(SEQ ID NO: 13)

Accordingly, a general formula for an active portion (e.g., ligand binding) of ActRIIA is a polypeptide that comprises, consists essentially of, or consists of amino acids 30-110 of SEQ ID NO: 9. Therefore ActRIIA polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIA beginning at a residue corresponding to any one of amino acids 21-30 (e.g., beginning at any one of amino acids 21, 22, 23, 24, 25, 26, 27, 28, 29, or 30) of SEQ ID NO: 9 and ending at a position corresponding to any one amino acids 110-135 (e.g., ending at any one of amino acids 110, 111, 112, 113, 114, 115, 116, 117, 118, 119, 120, 121, 122, 123, 124, 125, 126, 127, 128, 129, 130, 131, 132, 133, 134, or 135) of SEQ ID NO: 9. Other examples include constructs that begin at a position selected from 21-30 (e.g., beginning at any one of amino acids 21, 22, 23, 24, 25, 26, 27, 28, 29, or 30), 22-30 (e.g., beginning at any one of amino acids 22, 23, 24, 25, 26, 27, 28, 29, or 30), 23-30 (e.g., beginning at any one of amino acids 23, 24, 25, 26, 27, 28, 29, or 30), 24-30 (e.g., beginning at any one of amino acids 24, 25, 26, 27, 28, 29, or 30) of SEQ ID NO: 9, and end at a position selected from 111-135 (e.g., ending at any one of amino acids 111, 112, 113, 114, 115, 116, 117, 118, 119, 120, 121, 122, 123, 124, 125, 126, 127, 128, 129, 130, 131, 132, 133, 134 or 135), 112-135 (e.g., ending at any one of amino acids 112, 113, 114, 115, 116, 117, 118, 119, 120, 121, 122, 123, 124, 125, 126, 127, 128, 129, 130, 131, 132, 133, 134 or 135), 113-135 (e.g., ending at any one of amino acids 113, 114, 115, 116, 117, 118, 119, 120, 121, 122, 123, 124, 125, 126, 127, 128, 129, 130, 131, 132, 133, 134 or 135), 120-135 (e.g., ending at any one of amino acids 120, 121, 122, 123, 124, 125, 126, 127, 128, 129, 130, 131, 132, 133, 134 or 135),130-135 (e.g., ending at any one of amino acids 130, 131, 132, 133, 134 or 135), 111-134 (e.g., ending at any one of amino acids 110, 111, 112, 113, 114, 115, 116, 117, 118, 119, 120, 121, 122, 123, 124, 125, 126, 127, 128, 129, 130, 131, 132, 133, or 134), 111-133 (e.g., ending at any one of amino acids 110, 111, 112, 113, 114, 115, 116, 117, 118, 119, 120, 121, 122, 123, 124, 125, 126, 127, 128, 129, 130, 131, 132, or 133), 111-132 (e.g., ending at any one of amino acids 110, 111, 112, 113, 114, 115, 116, 117, 118, 119, 120, 121, 122, 123, 124, 125, 126, 127, 128, 129, 130, 131, or 132), or 111-131 (e.g., ending at any one of amino acids 110, 111, 112, 113, 114, 115, 116, 117, 118, 119, 120, 121, 122, 123, 124, 125, 126, 127, 128, 129, 130, or 131) of SEQ ID NO: 9. Variants within these ranges are also contemplated, particularly those comprising, consisting essentially of, or consisting of an amino acid sequence that has at least 70%, 75%, 80%, 85%, 86%, 87%, 88%, 89%, 90%, 91%, 92%, 93%, 94%, 95%, 96%, 97%, 98%, 99%, or 100% identity to the corresponding portion of SEQ ID NO: 9. Thus, in some embodiments, an ActRIIA polypeptide may comprise, consists essentially of, or consist of a polypeptide that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 9. Optionally, ActRIIA polypeptides comprise a polypeptide that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 9, and comprising no more than 1, 2, 5, 10 or 15 conservative amino acid changes in the ligand-binding pocket.

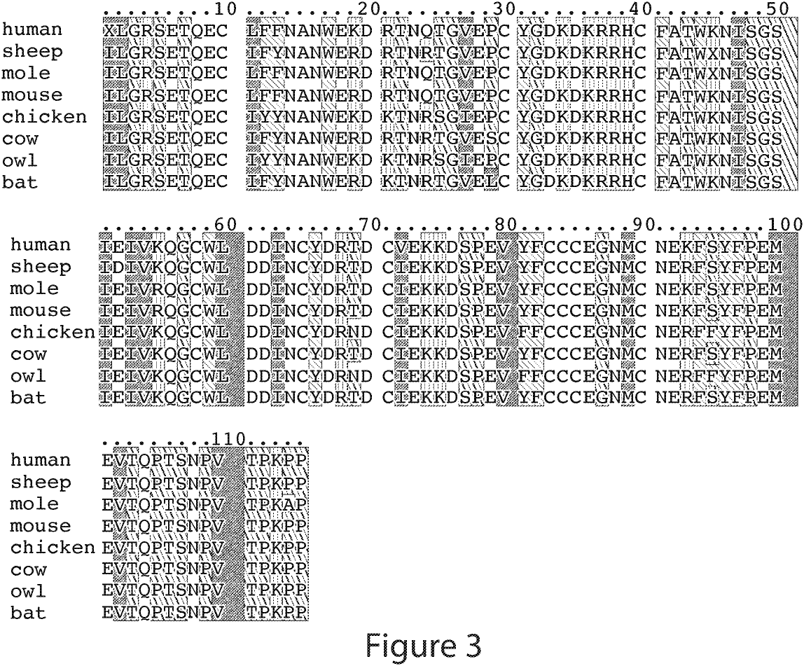

ActRIIA is well-conserved among vertebrates, with large stretches of the extracellular domain completely conserved. For example, FIG. 3 depicts a multi-sequence alignment of a human ActRIIA extracellular domain compared to various ActRIIA orthologs. Many of the ligands that bind to ActRIIA are also highly conserved. Accordingly, from these alignments, it is possible to predict key amino acid positions within the ligand-binding domain that are important for normal ActRIIA-ligand binding activities as well as to predict amino acid positions that are likely to be tolerant to substitution without significantly altering normal ActRIIA-ligand binding activities. Therefore, an active, human ActRIIA variant polypeptide useful in accordance with the presently disclosed methods may include one or more amino acids at corresponding positions from the sequence of another vertebrate ActRIIA, or may include a residue that is similar to that in the human or other vertebrate sequences.

Without meaning to be limiting, the following examples illustrate this approach to defining an active ActRIIA variant. As illustrated in FIG. 3, F13 in the human extracellular domain is Y in Ovisaries (SEQ ID NO: 76), Gallusgallus (SEQ ID NO: 79), BosTaurus (SEQ ID NO: 80), Tytoalba (SEQ ID NO: 81), and Myotisdavidii (SEQ ID NO: 82) ActRIIA, indicating that aromatic residues are tolerated at this position, including F, W, and Y. Q24 in the human extracellular domain is R in BosTaurus ActRIIA, indicating that charged residues will be tolerated at this position, including D, R, K, H, and E. S95 in the human extracellular domain is F in Gallusgallus and Tytoalba ActRIIA, indicating that this site may be tolerant of a wide variety of changes, including polar residues, such as E, D, K, R, H, S, T, P, G, Y, and probably hydrophobic residues such as L, I, or F. E52 in the human extracellular domain is D in Ovisaries ActRIIA, indicating that acidic residues are tolerated at this position, including D and E. P29 in the human extracellular domain is relatively poorly conserved, appearing as S in Ovisaries ActRIIA and L in Myotisdavidii ActRIIA, thus essentially any amino acid should be tolerated at this position.

In certain embodiments, the disclosure relates to single-arm heteromultimers that comprise at least one ActRIIA polypeptide, which includes fragments, functional variants, and modified forms thereof. Preferably, ActRIIA polypeptides for use in accordance with inventions of the disclosure (e.g., single-arm heteromultimers comprising an ActRIIA polypeptide and uses thereof) are soluble (e.g., an extracellular domain of ActRIIA). In other preferred embodiments, ActRIIA polypeptides for use in accordance with the inventions of the disclosure bind to and/or inhibit (antagonize) activity (e.g., induction of Smad signaling) of one or more TGF-beta superfamily ligands. In some embodiments, single-arm heteromultimers of the disclosure comprise at least one ActRIIA polypeptide that is at least 70%, 75%, 80%, 85%, 90%, 91%, 92%, 93%, 94%, 95%, 96%, 97%, 98%, or 99% identical to the amino acid sequence of any one of SEQ ID NOs: 9, 10, 11, 55, 57, 58, or 59. In some embodiments, single-arm heteromultimers of the disclosure comprise, consist, or consist essentially of at least one ActRIIA polypeptide that is at least 70%, 75%, 80%, 85%, 90%, 91%, 92%, 93%, 94%, 95%, 97%, 98%, or 99% identical to the amino acid sequence of any one of SEQ ID NOs: 9, 10, 11,55, 57, 58, or 59.

In some embodiments, the present disclosure contemplates making functional variants by modifying the structure of an ActRIIA or ActRIIB polypeptide for such purposes as enhancing therapeutic efficacy or stability (e.g., shelf-life and resistance to proteolytic degradation in vivo). Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the disclosure results in a functional homolog can be readily determined by assessing the ability of the variant polypeptide to produce a response in cells in a fashion similar to the wild-type polypeptide, or to bind to one or more ActRIIA or ActRIIB ligands including, for example, Activin A, activin B, GDF11, GDF8, GDF3, BMP5, BMP6, and BMP10.

In certain embodiments, the present disclosure contemplates specific mutations of an ActRIIA or ActRIIB polypeptide of the disclosure so as to alter the glycosylation of the polypeptide. Such mutations may be selected so as to introduce or eliminate one or more glycosylation sites, such as O-linked or N-linked glycosylation sites. Asparagine-linked glycosylation recognition sites generally comprise a tripeptide sequence, asparagine-X-threonine or asparagine-X-serine (where “X” is any amino acid) which is specifically recognized by appropriate cellular glycosylation enzymes. The alteration may also be made by the addition of, or substitution by, one or more serine or threonine residues to the sequence of the polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on a polypeptide is by chemical or enzymatic coupling of glycosides to the polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. Removal of one or more carbohydrate moieties present on a polypeptide may be accomplished chemically and/or enzymatically. Chemical deglycosylation may involve, for example, exposure of a polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Enzymatic cleavage of carbohydrate moieties on polypeptides can be achieved by the use of a variety of endo- and exo-glycosidases as described by Thotakura et al. [Meth. Enzymol. (1987) 138:350]. The sequence of a polypeptide may be adjusted, as appropriate, depending on the type of expression system used, as mammalian, yeast, insect, and plant cells may all introduce differing glycosylation patterns that can be affected by the amino acid sequence of the peptide. In general, single-arm ActRIIA heteromultimers or single-arm ActRIIB heteromultimers of the present disclosure for use in humans may be expressed in a mammalian cell line that provides proper glycosylation, such as HEK293 or CHO cell lines, although other mammalian expression cell lines are expected to be useful as well.

In certain embodiments, the present disclosure contemplates specific mutations of an ActRIIA or ActRIIB polypeptide of the disclosure. In some embodiments, one or more amino acid residues of a polypeptide of the present disclosure can be modified. In some embodiments, a modification is a glycosylated amino acid. In some embodiments, a modification is a PEGylated amino acid. In some embodiments, a modification is a famesylated amino acid. In some embodiments, a modification is an acetylated amino acid. In some embodiments, a modification is a biotinylated amino acid. In some embodiments, a modification is an amino acid conjugated to a lipid moiety. In some embodiments, a modification is an amino acid conjugated to an organic derivatizing agent. In some embodiments, a first and/or a second polypeptide of the present disclosure comprises one or more amino acid modifications selected from: a glycosylated amino acid, a PEGylated amino acid, a famesylated amino acid, an acetylated amino acid, a biotinylated amino acid, an amino acid conjugated to a lipid moiety, and an amino acid conjugated to an organic derivatizing agent. In some embodiments, a first and/or second polypeptide is glycosylated and has a glycosylation pattern obtainable from expression of the first and/or second polypeptide in a CHO cell.

The present disclosure further contemplates a method of generating mutants, particularly sets of combinatorial mutants of an ActRIIA or ActRIIB polypeptide of the present disclosure, as well as truncation mutants. Pools of combinatorial mutants are especially useful for identifying ActRIIA or ActRIIB polypeptide sequences. The purpose of screening such combinatorial libraries may be to generate, for example, polypeptides variants which have altered properties, such as altered pharmacokinetic or altered ligand binding. A variety of screening assays are provided below, and such assays may be used to evaluate variants. For example, ActRIIA or ActRIIB polypeptide variants may be screened for ability to bind to an ActRIIA or ActRIIB ligand (e.g., activin A, activin B, GDF11, GDF8, GDF3, BMP5, BMP6, and BMP10), to prevent binding of an ActRIIA or ActRIIB ligand to an ActRIIA or ActRIIB polypeptide, and/or to interfere with signaling caused by an ActRIIA or ActRIIB ligand. In some embodiments, a heteromultimer of the present disclosure inhibits activity of one or more ActRIIA or ActRIIB ligands in a cell-based assay.

The activity of an ActRIIA or ActRIIB single-arm heteromultimer of the disclosure also may be tested in a cell-based or in vivo assay. For example, the effect of a single-arm heteromultimer on the expression of genes involved in pulmonary hypertension in a pulmonary endothelial cell may be assessed. This may, as needed, be performed in the presence of one or more recombinant ActRIIA or ActRIIB ligand proteins (e.g., activin A, activin B, GDF11, GDF8, GDF3, BMP5, BMP6, and BMP10), and cells may be transfected so as to produce single-arm ActRIIA heteromultimer or a single-arm ActRIIB heteromultimer, and optionally, an ActRIIA or ActRIIB ligand. Likewise, a single-arm heteromultimer of the disclosure may be administered to a mouse or other animal, and one or more measurements, such as pulmonary hypertension may be assessed using art-recognized methods. Similarly, the activity of an ActRIIA or ActRIIB polypeptide or its variants may be tested in osteoblasts, adipocytes, and/or neuronal cells for any effect on growth of these cells, for example, by the assays as described herein and those of common knowledge in the art. A SMAD-responsive reporter gene may be used in such cell lines to monitor effects on downstream signaling.

Combinatorial-derived variants can be generated which have increased selectivity or generally increased potency relative to a reference single-arm ActRIIA heteromultimer or a single-arm ActRIIB heteromultimer. Such variants, when expressed from recombinant DNA constructs, can be used in gene therapy protocols. Likewise, mutagenesis can give rise to variants which have extracellular half-lives dramatically different than the corresponding unmodified single-arm ActRIIA heteromultimer or single-arm ActRIIB heteromultimer. For example, the altered protein can be rendered either more stable or less stable to proteolytic degradation or other cellular processes which result in destruction, or otherwise inactivation, of an unmodified polypeptide. Such variants, and the genes which encode them, can be utilized to alter polypeptide complex levels by modulating the half-life of the polypeptide. For instance, a short half-life can give rise to more transient biological effects and, when part of an inducible expression system, can allow tighter control of recombinant polypeptide complex levels outside the cell. In an Fc fusion protein, mutations may be made in the linker (if any) and/or the Fc portion to alter the half-life of the single-arm ActRIIA heteromultimer or single-arm ActRIIB heteromultimer.

A combinatorial library may be produced by way of a degenerate library of genes encoding a library of polypeptides which each include at least a portion of potential ActRIIA or ActRIIB sequences. For instance, a mixture of synthetic oligonucleotides can be enzymatically ligated into gene sequences such that the degenerate set of potential ActRIIA or ActRIIB encoding nucleotide sequences are expressible as individual polypeptides, or alternatively, as a set of larger fusion proteins (e.g., for phage display).

There are many ways by which the library of potential homologs can be generated from a degenerate oligonucleotide sequence. Chemical synthesis of a degenerate gene sequence can be carried out in an automatic DNA synthesizer, and the synthetic genes can then be ligated into an appropriate vector for expression. The synthesis of degenerate oligonucleotides is well known in the art. See, e.g., Narang, SA (1983) Tetrahedron 39:3; Itakura et al. (1981) Recombinant DNA, Proc. 3rd Cleveland Sympos. Macromolecules, ed. AG Walton, Amsterdam: Elsevier pp273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins. See, e.g., Scott et al., (1990) Science 249:386-390; Roberts et al. (1992) PNAS USA 89:2429-2433; Devlin et al. (1990) Science 249: 404-406; Cwirla et al., (1990) PNAS USA 87: 6378-6382; as well as U.S. Pat. Nos: 5,223,409, 5,198,346, and 5,096,815.

Alternatively, other forms of mutagenesis can be utilized to generate a combinatorial library. For example, single-arm ActRIIA heteromultimers or single-arm ActRIIB heteromultimers of the disclosure can be generated and isolated from a library by screening using, for example, alanine scanning mutagenesis [see, e.g., Ruf et al. (1994) Biochemistry 33:1565-1572; Wang et al. (1994) J. Biol. Chem. 269:3095-3099; Balint et al. (1993) Gene 137:109-118; Grodberg et al. (1993) Eur. J. Biochem. 218:597-601; Nagashima et al. (1993) J. Biol. Chem. 268:2888-2892; Lowman et al. (1991) Biochemistry 30:10832-10838; and Cunningham et al. (1989) Science 244:1081-1085], by linker scanning mutagenesis [see, e.g., Gustin et al. (1993) Virology 193:653-660; and Brown et al. (1992) Mol. Cell Biol. 12:2644-2652; McKnight et al. (1982) Science 232:316], by saturation mutagenesis [see, e.g., Meyers et al., (1986) Science 232:613]; by PCR mutagenesis [see, e.g., Leung et al. (1989) Method Cell Mol Biol 1:11-19]; or by random mutagenesis, including chemical mutagenesis [see, e.g., Miller et al. (1992) A Short Course in Bacterial Genetics, CSHL Press, Cold Spring Harbor, NY; and Greener et al. (1994) Strategies in Mol Biol 7:32-34]. Linker scanning mutagenesis, particularly in a combinatorial setting, is an attractive method for identifying truncated (bioactive) forms of ActRIIA or ActRIIB polypeptides.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of single-arm ActRIIA heteromultimers or single-arm ActRIIB heteromultimers of the disclosure. The most widely used techniques for screening large gene libraries typically comprise cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Preferred assays include binding assays and/or cell-signaling assays for ActRIIA or ActRIIB ligands (e.g., Activin A, activin B, GDF11, GDF8, GDF3, BMP5, BMP6, and BMP10).

In certain embodiments, single-arm ActRIIA heteromultimers or single-arm ActRIIB heteromultimers of the disclosure may further comprise post-translational modifications in addition to any that are naturally present in the ActRIIA or ActRIIB polypeptide. Such modifications include, but are not limited to, acetylation, carboxylation, glycosylation, phosphorylation, lipidation, and acylation. As a result, the ActRIIA or ActRIIB single-arm heteromultimer may comprise non-amino acid elements, such as polyethylene glycols, lipids, polysaccharide or monosaccharide, and phosphates. Effects of such non-amino acid elements on the functionality of a single-arm heteromultimer may be tested as described herein for other single-arm heteromultimer variants. When a polypeptide of the disclosure is produced in cells by cleaving a nascent form of the polypeptide, post-translational processing may also be important for correct folding and/or function of the protein. Different cells (e.g., CHO, HeLa, MDCK, 293, WI38, NIH-3T3 or HEK293) have specific cellular machinery and characteristic mechanisms for such post-translational activities and may be chosen to ensure the correct modification and processing of the ActRIIA or ActRIIB polypeptide.

In certain aspects, the polypeptides disclosed herein may form protein heteromultimers comprising at least one ActRIIA or ActRIIB polypeptide associated, covalently or non-covalently, with at least one polypeptide comprising a complementary member of an interaction pair. Preferably, polypeptides disclosed herein form single-arm heterodimers, although higher order heteromultimers are also included such as, but not limited to, heterotrimers, heterotetramers, and further oligomeric structures. In some embodiments, ActRIIA or ActRIIB polypeptides of the present disclosure comprise at least one multimerization domain. As disclosed herein, the term “multimerization domain” refers to an amino acid or sequence of amino acids that promote covalent or non-covalent interaction between at least a first polypeptide and at least a second polypeptide. Polypeptides disclosed herein may be joined covalently or non-covalently to a multimerization domain. Preferably, a multimerization domain promotes interaction between a single-arm polypeptide (e.g., a fusion polypeptide comprising an ActRIIA or ActRIIB polypeptide) and a complementary member of an interaction pair to promote heteromultimer formation (e.g., heterodimer formation), and optionally hinders or otherwise disfavors homomultimer formation (e.g., homodimer formation), thereby increasing the yield of desired heteromultimer.

Many methods known in the art can be used to generate single-arm ActRIIA heteromultimers or single-arm ActRIIB heteromultimers of the disclosure. For example, non-naturally occurring disulfide bonds may be constructed by replacing on a first polypeptide (e.g., a fusion polypeptide comprising an ActRIIA or ActRIIB polypeptide) a naturally occurring amino acid with a free thiol-containing residue, such as cysteine, such that the free thiol interacts with another free thiol-containing residue on a second polypeptide (e.g., a complementary member of an interaction pair) such that a disulfide bond is formed between the first and second polypeptides. Additional examples of interactions to promote heteromultimer formation include, but are not limited to, ionic interactions such as described in Kjaergaard et al., WO2007147901; electrostatic steering effects such as described in Kannan et al., U.S.8,592,562; coiled-coil interactions such as described in Christensen et al., U.S.20120302737; leucine zippers such as described in Pack & Plueckthun,(1992) Biochemistry 31: 1579-1584; and helix-turn-helix motifs such as described in Pack et al., (1993) Bio/Technology 11: 1271-1277. Linkage of the various segments may be obtained via, e.g., covalent binding such as by chemical cross-linking, peptide linkers, disulfide bridges, etc., or affinity interactions such as by avidin-biotin or leucine zipper technology.

In certain aspects, a multimerization domain may comprise one component of an interaction pair. In some embodiments, the polypeptides disclosed herein may form heteromultimers comprising a first polypeptide covalently or non-covalently associated with a second polypeptide, wherein the first polypeptide comprises the amino acid sequence of an ActRIIA or ActRIIB polypeptide and the amino acid sequence of a first member of an interaction pair; and the second polypeptide comprises the amino acid sequence of a second member of an interaction pair. The interaction pair may be any two polypeptide sequences that interact to form a heteromultimer, particularly a heterodimer, although operative embodiments may also employ an interaction pair that can form a homodimeric complex. One member of the interaction pair may be fused to an ActRIIA or ActRIIB polypeptide as described herein, including for example, a polypeptide sequence comprising, consisting essentially of, or consisting of an amino acid sequence that is at least 80%, 85%, 90%, 91%, 92%, 93%, 94%, 95%, 96%, 97%, 98%, 99%, or 100% identical to the sequence of any one of SEQ ID NOs: 1, 2, 3, 4, 5, 6, 9, 10, 11, . An interaction pair may be selected to confer an improved property/activity such as increased serum half-life, or to act as an adaptor on to which another moiety is attached to provide an improved property/activity. For example, a polyethylene glycol moiety may be attached to one or both components of an interaction pair to provide an improved property/activity such as improved serum half-life.

The first and second members of the interaction pair may be an asymmetric pair, meaning that the members of the pair preferentially associate with each other rather than self-associate. Accordingly, first and second members of an asymmetric interaction pair may associate to form a heterodimeric interaction-pair. Alternatively, the interaction pair may be unguided, meaning that the members of the pair may associate with each other or self-associate without substantial preference and thus may have the same or different amino acid sequences. Accordingly, first and second members of an unguided interaction pair may associate to form a homodimer interaction-pair or a heterodimeric action-pair. Optionally, the first member of the interaction pair (e.g., an asymmetric pair or an unguided interaction pair) associates covalently with the second member of the interaction pair. Optionally, the first member of the interaction pair (e.g., an asymmetric pair or an unguided interaction pair) associates non-covalently with the second member of the interaction pair.

As specific examples, the present disclosure provides heteromultimer fusion proteins comprising at least one ActRIIA or ActRIIB polypeptide fused to a polypeptide. In some embodiments, the present disclosure provides heteromultimer fusion proteins comprising at least one ActRIIA or ActRIIB polypeptide fused to a polypeptide comprising a constant region of an immunoglobulin, such as a CH1, CH2, or CH3 domain of an immunoglobulin or an Fc domain. Fc domains derived from human IgG1, IgG2, IgG3, and IgG4 are provided herein. In some embodiments, the first constant region from an IgG heavy chain is a first immunoglobulin Fc domain. In some embodiments, a second constant region from an IgG heavy chain is a first immunoglobulin Fc domain. In some embodiments, Fc domains are derived from the constant region from a human IgG1, IgG2, IgG3, or IgG4 heavy chain. In some embodiments, Fc domains comprise the constant region from a human IgG1. In some embodiments, Fc domains comprise the constant region from a human IgG2. In some embodiments, Fc domains comprise the constant region from a human IgG3. In some embodiments, Fc domains comprise the constant region from a human IgG4.

Other mutations are known that decrease either CDC or ADCC activity, and collectively, any of these variants are included in the disclosure and may be used as advantageous components of a single-arm heteromultimer fusion protein of the disclosure. Optionally, the IgG1 Fc domain of SEQ ID NO: 22 has one or more mutations at residues such as Asp-265, Lys-322, and Asn-434 (numbered in accordance with the corresponding full-length IgG1). In certain cases, the mutant Fc domain having one or more of these mutations (e.g., Asp-265 mutation) has reduced ability of binding to the Fcγ receptor relative to a wildtype Fc domain. In other cases, the mutant Fc domain having one or more of these mutations (e.g., Asn-434 mutation) has increased ability of binding to the MHC class I-related Fc-receptor (FcRN) relative to a wildtype Fc domain.

An example of a native amino acid sequence that may be used for the Fc portion of human IgG1 (G1Fc) is shown below (SEQ ID NO: 22). Dotted underline indicates the hinge region, and solid underline indicates positions with naturally occurring variants. In part, the disclosure provides polypeptides comprising amino acid sequences with 80%, 85%, 90%, 91%, 92%, 93%, 94%, 95%, 96%, 97%, 98%, or 99% identity to SEQ ID NO: 22. Naturally occurring variants in G1Fc would include E134D and M136L according to the numbering system used in SEQ ID NO: 22 (see UniProt P01857).

1 THTCPPCPAP ELLGGPSVFL FPPKPKDTLM ISRTPEVTCV VVDVSHEDPE
51 VKFNWYVDGV EVHNAKTKPR EEQYNSTYRV VSVLTVLHQD WLNGKEYKCK
101 VSNKALPAPI EKTISKAKGQ PREPQVYTLP PSREEMTKNQ VSLTCLVKGF
151 YPSDIAVEWE SNGQPENNYK TTPPVLDSDG SFFLYSKLTV DKSRWQQGNV
201 FSCSVMHEAL HNHYTQKSLS LSPGK (SEQ ID NO: 22)

An example of a native amino acid sequence that may be used for the Fc portion of human IgG2 (G2Fc) is shown below (SEQ ID NO: 23). Dotted underline indicates the hinge region and double underline indicates positions where there are database conflicts in the sequence (according to UniProt P01859). In part, the disclosure provides polypeptides comprising amino acid sequences with 80%, 85%, 90%, 91%, 92%, 93%, 94%, 95%, 96%, 97%, 98%, or 99% identity to SEQ ID NO: 23.

1 VECPPCPAPP VAGPSVFLFP PKPKDTLMIS RTPEVTCVVV DVSHEDPEVQ
51 FNWYVDGVEV HNAKTKPREE QFNSTFRVVS VLTVVHQDWL NGKEYKCKVS
101 NKGLPAPIEK TISKTKGQPR EPQVYTLPPS REEMTKNQVS LTCLVKGFYP
151 SDIAVEWESN GQPENNYKTT PPMLDSDGSF FLYSKLTVDK SRWQQGNVFS
201 CSVMHEALHN HYTQKSLSLS PGK (SEQ ID NO: 23)

Two examples of amino acid sequences that may be used for the Fc portion of human IgG3 (G3Fc) are shown below. The hinge region in G3Fc can be up to four times as long as in other Fc chains and contains three identical 15-residue segments preceded by a similar 17-residue segment. The first G3Fc sequence shown below (SEQ ID NO: 24) contains a short hinge region consisting of a single 15-residue segment, whereas the second G3Fc sequence (SEQ ID NO: 25) contains a full-length hinge region. In each case, dotted underline indicates the hinge region, and solid underline indicates positions with naturally occurring variants according to UniProt P01859. In part, the disclosure provides polypeptides comprising amino acid sequences with 80%, 85%, 90%, 91%, 92%, 93%, 94%, 95%, 96%, 97%, 98%, or 99% identity to SEQ ID NOs: 24 and 25.

1 EPKSCDTPPP CPRCPAPELL GGPSVFLFPP KPKDTLMISR TPEVTCVVVD
51 VSHEDPEVQF KWYVDGVEVH NAKTKPREEQ YNSTFRWSV LTVLHQDWLN
101 GKEYKCKVSN KALPAPIEKT ISKTKGQPRE PQVYTLPPSR EEMTKNQVSL
151 TCLVKGFYPS DIAVEWESSG QPENNYNTTP PMLDSDGSFF LYSKLTVDKS
201 RWQQGNIFSC SVMHEALHNR FTQKSLSLSP GK (SEQ ID NO: 24)

1 ELKTPLGDTT HTCPRCPEPK SCDTPPPCPR CPEPKSCDTP PPCPRCPEPK
51 SCDTPPPCPR CPAPELLGGP SVFLFPPKPK DTLMISRTPE VTCVVVDVSH
101 EDPEVQFKWY VDGVEVHNAK TKPREEQYNS TFRVVSVLTV LHQDWLNGKE
151 YKCKVSNKAL PAPIEKTISK TKGQPREPQV YTLPPSREEM TKNQVSLTCL
201 VKGFYPSDIA VEWESSGQPE NNYNTTPPML DSDGSFFLYS KLTVDKSRWQ
251 QGNIFSCSVM HEALHNRFTQ KSLSLSPGK (SEQ ID NO: 25)

Naturally occurring variants in G3Fc (for example, see Uniprot P01860) include E68Q, P76L, E79Q, Y81F, D97N, N100D, T124A, S169N, S169del, F221Y when converted to the numbering system used in SEQ ID NO: 24, and the present disclosure provides fusion proteins comprising G3Fc domains containing one or more of these variations. In addition, the human immunoglobulin IgG3 gene (IGHG3) shows a structural polymorphism characterized by different hinge lengths [see Uniprot P01859]. Specifically, variant WIS is lacking most of the V region and all of the CH1 region. It has an extra interchain disulfide bond at position 7 in addition to the 11 normally present in the hinge region. Variant ZUC lacks most of the V region, all of the CH1 region, and part of the hinge. Variant OMM may represent an allelic form or another gamma chain subclass. The present disclosure provides additional fusion proteins comprising G3Fc domains containing one or more of these variants.

An example of a native amino acid sequence that may be used for the Fc portion of human IgG4 (G4Fc) is shown below (SEQ ID NO: 26). Dotted underline indicates the hinge region. In part, the disclosure provides polypeptides comprising amino acid sequences with 80%, 85%, 90%, 91%, 92%, 93%, 94%, 95%, 96%, 97%, 98%, or 99% identity to SEQ ID NO: 26.

1 ESKYGPPCPS CPAPEFLGGP SVFLFPPKPK DTLMISRTPE VTCVVVDVSQ
51 EDPEVQFNWY VDGVEVHNAK TKPREEQFNS TYRVVSVLTV LHQDWLNGKE
101 YKCKVSNKGL PSSIEKTISK AKGQPREPQV YTLPPSQEEM TKNQVSLTCL
151 VKGFYPSDIA VEWESNGQPE NNYKTTPPVL DSDGSFFLYS RLTVDKSRWQ
201 EGNVFSCSVM HEALHNHYTQ KSLSLSLGK (SEQ ID NO: 26)

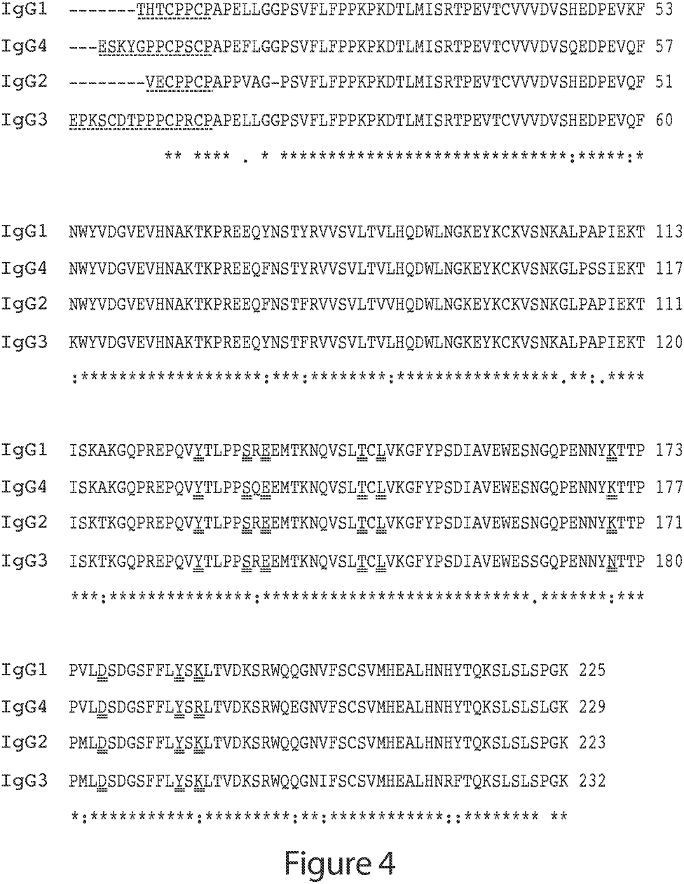

A variety of engineered mutations in the Fc domain are presented herein with respect to the G1Fc sequence (SEQ ID NO: 22), and analogous mutations in G2Fc, G3Fc, and G4Fc can be derived from their alignment with G1Fc in FIG. 4. Due to unequal hinge lengths, analogous Fc positions based on isotype alignment (FIG. 4) possess different amino acid numbers in SEQ ID NOs: 22, 23, 24, and 26. It can also be appreciated that a given amino acid position in an immunoglobulin sequence consisting of hinge, C_{H}2, and C_{H}3 regions (e.g., SEQ ID NOs: 22, 23, 24, and 26) will be identified by a different number than the same position when numbering encompasses the entire IgG1 heavy-chain constant domain (consisting of the C_{H}1, hinge, C_{H}2, and C_{H}3 regions) as in the Uniprot database. For example, correspondence between selected C_{H}3 positions in a human G1Fc sequence (SEQ ID NO: 22), the human IgG1 heavy chain constant domain (Uniprot P01857), and the human IgG1 heavy chain is as follows.

Correspondence of C_{H}3 Positions in Different Numbering Systems

| G1Fc (Numbering begins at first threonine in hinge region) | IgG1 heavy chain constant domain (Numbering begins at C_{H}1) | IgG1 heavy chain (EU numbering scheme of Kabat et al., 1991*) |
|---|---|---|
| Y127 | Y232 | Y349 |
| S132 | S237 | S354 |
| E134 | E239 | E356 |
| T144 | T249 | T366 |
| L146 | L251 | L368 |
| K170 | K275 | K392 |
| D177 | D282 | D399 |
| Y185 | Y290 | Y407 |
| K187 | K292 | K409 |

* Kabat et al. (eds) 1991; pp. 688-696 in Sequences of Proteins of Immunological Interest, 5^{th} ed., Vol. 1, NIH, Bethesda, MD.

A problem that arises in large-scale production of asymmetric immunoglobulin-based proteins from a single cell line is known as the “chain association issue”. As confronted prominently in the production of bispecific antibodies, the chain association issue concerns the challenge of efficiently producing a desired multichain protein from among the multiple combinations that inherently result when different heavy chains and/or light chains are produced in a single cell line [see, for example, Klein et al (2012) mAbs 4:653-663]. This problem is most acute when two different heavy chains and two different light chains are produced in the same cell, in which case there are a total of 16 possible chain combinations (although some of these are identical) when only one is typically desired. Nevertheless, the same principle accounts for diminished yield of a desired multichain fusion protein that incorporates only two different (asymmetric) heavy chains.

Various methods are known in the art that increase desired pairing of Fc-containing fusion polypeptide chains in a single cell line to produce a preferred asymmetric fusion protein at acceptable yields [see, for example, Klein et al (2012) mAbs 4:653-663]. Methods to obtain desired pairing of Fc-containing chains include, but are not limited to, charge-based pairing (electrostatic steering), “knobs-into-holes” steric pairing, SEEDbody pairing, and leucine zipper-based pairing. See, for example, Ridgway et al (1996) Protein Eng 9:617-621; Merchant et al (1998) Nat Biotech 16:677-681; Davis et al (2010) Protein Eng Des Sel 23:195-202; Gunasekaran et al (2010); 285:19637-19646; Wranik et al (2012) J Biol Chem 287:43331-43339; US5932448; WO 1993/011162; WO 2009/089004, and WO 2011/034605.

For example, one means by which interaction between specific polypeptides may be promoted is by engineering protuberance-into-cavity (knob-into-holes) complementary regions such as described in Arathoon et al., U.S.7,183,076 and Carter et al., U.S.5,731,168. “Protuberances” are constructed by replacing small amino acid side chains from the interface of the first polypeptide (e.g., a first interaction pair) with larger side chains (e.g., tyrosine or tryptophan). Complementary “cavities” of identical or similar size to the protuberances are optionally created on the interface of the second polypeptide (e.g., a second interaction pair) by replacing large amino acid side chains with smaller ones (e.g., alanine or threonine). Where a suitably positioned and dimensioned protuberance or cavity exists at the interface of either the first or second polypeptide, it is only necessary to engineer a corresponding cavity or protuberance, respectively, at the adjacent interface.

At neutral pH (7.0), aspartic acid and glutamic acid are negatively charged, and lysine, arginine, and histidine are positively charged. These charged residues can be used to promote heterodimer formation and at the same time hinder homodimer formation. Attractive interactions take place between opposite charges and repulsive interactions occur between like charges. In part, protein complexes disclosed herein make use of the attractive interactions for promoting heteromultimer formation (e.g., heterodimer formation), and optionally repulsive interactions for hindering homodimer formation (e.g., homodimer formation) by carrying out site directed mutagenesis of charged interface residues.

For example, the IgG1 CH3 domain interface comprises four unique charge residue pairs involved in domain-domain interactions: Asp356-Lys439′, Glu357-Lys370′, Lys392-Asp399′, and Asp399-Lys409′ [residue numbering in the second chain is indicated by (’)]. It should be noted that the numbering scheme used here to designate residues in the IgG1 CH3 domain conforms to the EU numbering scheme of Kabat. Due to the 2-fold symmetry present in the CH3-CH3 domain interactions, each unique interaction will represented twice in the structure (e.g., Asp-399-Lys409’ and Lys409-Asp399’). In the wild-type sequence, K409-D399’ favors both heterodimer and homodimer formation. A single mutation switching the charge polarity (e.g., K409E; positive to negative charge) in the first chain leads to unfavorable interactions for the formation of the first chain homodimer. The unfavorable interactions arise due to the repulsive interactions occurring between the same charges (negative-negative; K409E-D399’ and D399-K409E’). A similar mutation switching the charge polarity (D399K’; negative to positive) in the second chain leads to unfavorable interactions (K409’-D399K’ and D399K-K409’) for the second chain homodimer formation. But, at the same time, these two mutations (K409E and D399K’) lead to favorable interactions (K409E-D399K’ and D399-K409’) for the heterodimer formation.

The electrostatic steering effect on heterodimer formation and homodimer discouragement can be further enhanced by mutation of additional charge residues which may or may not be paired with an oppositely charged residue in the second chain including, for example, Arg355 and Lys360. The table below lists possible charge change mutations that can be used, alone or in combination, to enhance heteromultimer formation of the heteromultimers disclosed herein.

Examples of Pair-Wise Charged Residue Mutations to Enhance Heterodimer Formation

| Position in first chain | Mutation in first chain | Interacting position in second chain | Corresponding mutation in second chain |
|---|---|---|---|
| Lys409 | Asp or Glu | Asp399’ | Lys, Arg, or His |
| Lys392 | Asp or Glu | Asp399’ | Lys, Arg, or His |
| Lys439 | Asp or Glu | Asp356’ | Lys, Arg, or His |
| Lys370 | Asp or Glu | Glu357’ | Lys, Arg, or His |
| Asp399 | Lys, Arg, or His | Lys409’ | Asp or Glu |
| Asp399 | Lys, Arg, or His | Lys392’ | Asp or Glu |
| Asp356 | Lys, Arg, or His | Lys439’ | Asp or Glu |
| Glu357 | Lys, Arg, or His | Lys370’ | Asp or Glu |

In some embodiments, one or more residues that make up the CH3-CH3 interface in a fusion protein of the instant application are replaced with a charged amino acid such that the interaction becomes electrostatically unfavorable. For example, a positive-charged amino acid in the interface (e.g., a lysine, arginine, or histidine) is replaced with a negatively charged amino acid (e.g., aspartic acid or glutamic acid). Alternatively, or in combination with the forgoing substitution, a negative-charged amino acid in the interface is replaced with a positive-charged amino acid. In certain embodiments, the amino acid is replaced with a non-naturally occurring amino acid having the desired charge characteristic. It should be noted that mutating negatively charged residues (Asp or Glu) to His will lead to increase in side chain volume, which may cause steric issues. Furthermore, His proton donor- and acceptor-form depends on the localized environment. These issues should be taken into consideration with the design strategy. Because the interface residues are highly conserved in human and mouse IgG subclasses, electrostatic steering effects disclosed herein can be applied to human and mouse IgG1, IgG2, IgG3, and IgG4. This strategy can also be extended to modifying uncharged residues to charged residues at the CH3 domain interface.

In part, the disclosure provides desired pairing of asymmetric Fc-containing polypeptide chains using Fc sequences engineered to be complementary on the basis of charge pairing (electrostatic steering). One of a pair of Fc sequences with electrostatic complementarity can be arbitrarily fused to the ActRIIA or ActRIIB polypeptide of the construct, with or without an optional linker, to generate an ActRIIA or ActRIIB fusion polypeptide This single chain can be coexpressed in a cell of choice along with the Fc sequence complementary to the first Fc to favor generation of the desired multichain construct (e.g., a single-arm ActRIIA heteromultimer or single-arm ActRIIB heteromultimer). In this example based on electrostatic steering, SEQ ID NO: 14 [human GIFc(E134K/D177K)] and SEQ ID NO: 15 [human G1Fc(K170D/K187D)] are examples of complementary Fc sequences in which the engineered amino acid substitutions are double underlined, and the ActRIIA or ActRIIB polypeptide of the construct can be fused to either SEQ ID NO: 14 or SEQ ID NO: 15, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 4) will generate complementary Fc pairs which may be used instead of the complementary hG1Fc pair below (SEQ ID NOs: 14 and 15).

1 THTCPPCPAP ELLGGPSVFL FPPKPKDTLM ISRTPEVTCV VVDVSHEDPE
51 VKFNWYVDGV EVHNAKTKPR EEQYNSTYRV VSVLTVLHQD WLNGKEYKCK
101 VSNKALPAPI EKTISKAKGQ PREPQVYTLP PSRKEMTKNQ VSLTCLVKGF
151 YPSDIAVEWE SNGQPENNYK TTPPVLKSDG SFFLYSKLTV DKSRWQQGNV
201 FSCSVMHEAL HNHYTQKSLS LSPGK (SEQ ID NO: 14)
1 THTCPPCPAP ELLGGPSVFL FPPKPKDTLM ISRTPEVTCV WDVSHEDPE
51 VKFNWYVDGV EVHNAKTKPR EEQYNSTYRV VSVLTVLHQD WLNGKEYKCK
101 VSNKALPAPI EKTISKAKGQ PREPQVYTLP PSREEMTKNQ VSLTCLVKGF
151 YPSDIAVEWE SNGQPENNYD TTPPVLDSDG SFFLYSDLTV DKSRWQQGNV
201 FSCSVMHEAL HNHYTQKSLS LSPGK (SEQ ID NO: 15)

In part, the disclosure provides desired pairing of asymmetric Fc-containing polypeptide chains using Fc sequences engineered for steric complementarity. In part, the disclosure provides knobs-into-holes pairing as an example of steric complementarity. One of a pair of Fc sequences with steric complementarity can be arbitrarily fused to the ActRIIA or ActRIIB polypeptide of the construct, with or without an optional linker, to generate a single-arm ActRIIB heteromultimer fusion construct or a single-arm ActRIIB heteromultimer fusion construct. This single chain can be coexpressed in a cell of choice along with the Fc sequence complementary to the first Fc to favor generation of the desired multichain construct (e.g., a single-arm ActRIIA heteromultimer or a single-arm ActRIIB heteromultimer). In this example based on knobs-into-holes pairing, SEQ ID NO: 16 [human G1Fc(T144Y)] and SEQ ID NO: 17 [human G1Fc(Y185T)] are examples of complementary Fc sequences in which the engineered amino acid substitutions are double underlined, and the ActRIIA or ActRIIB polypeptide of the construct can be fused to either SEQ ID NO: 16 or SEQ ID NO: 17, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 4) will generate complementary Fc pairs which may be used instead of the complementary hG1Fc pair below (SEQ ID NOs: 16 and 17).

1 THTCPPCPAP ELLGGPSVFL FPPKPKDTLM ISRTPEVTCV WDVSHEDPE
51 VKFNWYVDGV EVHNAKTKPR EEQYNSTYRV VSVLTVLHQD WLNGKEYKCK
101 VSNKALPAPI EKTISKAKGQ PREPQVYTLP PSREEMTKNQ VSLYCLVKGF
151 YPSDIAVEWE SNGQPENNYK TTPPVLDSDG SFFLYSKLTV DKSRWQQGNV
201 FSCSVMHEAL HNHYTQKSLS LSPGK (SEQ ID NO: 16)
1 THTCPPCPAP ELLGGPSVFL FPPKPKDTLM ISRTPEVTCV WDVSHEDPE
51 VKFNWYVDGV EVHNAKTKPR EEQYNSTYRV VSVLTVLHQD WLNGKEYKCK
101 VSNKALPAPI EKTISKAKGQ PREPQVYTLP PSREEMTKNQ VSLTCLVKGF
151 YPSDIAVEWE SNGQPENNYK TTPPVLDSDG SFFLTSKLTV DKSRWQQGNV
201 FSCSVMHEAL HNHYTQKSLS LSPGK (SEQ ID NO: 17)

An example of Fc complementarity based on knobs-into-holes pairing combined with an engineered disulfide bond is disclosed in SEQ ID NO: 18 [hG1Fc(S132C/T144W)] and SEQ ID NO: 19 [hG1Fc(Y127C/T144S/L146A/Y185V)]. The engineered amino acid substitutions in these sequences are double underlined, and the ActRIIA or ActRIIB polypeptide of the construct can be fused to either SEQ ID NO: 18 or SEQ ID NO: 19, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 4) will generate complementary Fc pairs which may be used instead of the complementary hG1Fc pair below (SEQ ID NOs: 18 and 19).

1 THTCPPCPAP ELLGGPSVFL FPPKPKDTLM ISRTPEVTCV WDVSHEDPE
51 VKFNWYVDGV EVHNAKTKPR EEQYNSTYRV VSVLTVLHQD WLNGKEYKCK
101 VSNKALPAPI EKTISKAKGQ PREPQVYTLP PCREEMTKNQ VSLWCLVKGF
151 YPSDIAVEWE SNGQPENNYK TTPPVLDSDG SFFLYSKLTV DKSRWQQGNV
201 FSCSVMHEAL HNHYTQKSLS LSPGK (SEQ ID NO: 18)
1 THTCPPCPAP ELLGGPSVFL FPPKPKDTLM ISRTPEVTCV WDVSHEDPE
51 VKFNWYVDGV EVHNAKTKPR EEQYNSTYRV VSVLTVLHQD WLNGKEYKCK
101 VSNKALPAPI EKTISKAKGQ PREPQVCTLP PSREEMTKNQ VSLSCAVKGF
151 YPSDIAVEWE SNGQPENNYK TTPPVLDSDG SFFLVSKLTV DKSRWQQGNV
201 FSCSVMHEAL HNHYTQKSLS LSPGK (SEQ ID NO: 19)

In part, the disclosure provides desired pairing of asymmetric Fc-containing polypeptide chains using Fc sequences engineered to generate interdigitating β-strand segments of human IgG and IgA C_{H}3 domains. Such methods include the use of strand-exchange engineered domain (SEED) C_{H}3 heterodimers allowing the formation of SEEDbody fusion proteins [see, for example, Davis et al (2010) Protein Eng Design Sel 23:195-202]. One of a pair of Fc sequences with SEEDbody complementarity can be arbitrarily fused to the ActRIIA or ActRIIB polypeptide of the construct, with or without an optional linker, to generate a single-arm ActRIIA heteromultimer fusion construct or a single-arm ActRIIB heteromultimer construct. This single chain can be coexpressed in a cell of choice along with the Fc sequence complementary to the first Fc to favor generation of the desired multichain construct. In this example based on SEEDbody (Sb) pairing, SEQ ID NO: 20 [hG1Fc(Sb_{AG})] and SEQ ID NO: 21 [hG1Fc(Sb_{GA})] are examples of complementary IgG Fc sequences in which the engineered amino acid substitutions from IgA Fc are double underlined, and the ActRIIA or ActRIIB polypeptide of the construct can be fused to either SEQ ID NO: 20 or SEQ ID NO: 21, but not both. Given the high degree of amino acid sequence identity between native hG1Fc, native hG2Fc, native hG3Fc, and native hG4Fc, it can be appreciated that amino acid substitutions at corresponding positions in hG1Fc, hG2Fc, hG3Fc, or hG4Fc (see FIG. 4) will generate an Fc monomer which may be used in the complementary IgG-IgA pair below (SEQ ID NOs: 20 and 21).

1 THTCPPCPAP ELLGGPSVFL FPPKPKDTLM ISRTPEVTCV WDVSHEDPE
51 VKFNWYVDGV EVHNAKTKPR EEQYNSTYRV VSVLTVLHQD WLNGKEYKCK
101 VSNKALPAPI EKTISKAKGQ PFRPEVHLLP PSREEMTKNQ VSLTCLARGF
151 YPKDIAVEWE SNGQPENNYK TTPSRQEPSQ GTTTFAVTSK LTVDKSRWQQ
201 GNVFSCSVMH EALHNHYTQK TISLSPGK (SEQ ID NO: 20)
1 THTCPPCPAP ELLGGPSVFL FPPKPKDTLM ISRTPEVTCV WDVSHEDPE
51 VKFNWYVDGV EVHNAKTKPR EEQYNSTYRV VSVLTVLHQD WLNGKEYKCK
101 VSNKALPAPI EKTISKAKGQ PREPQVYTLP PPSEELALNE LVTLTCLVKG
151 FYPSDIAVEW ESNGQELPRE KYLTWAPVLD SDGSFFLYSI LRVAAEDWKK
201 GDTFSCSVMH EALHNHYTQK SLDRSPGK (SEQ ID NO: 21)

In part, the disclosure provides desired pairing of asymmetric Fc-containing polypeptide chains with a cleavable leucine zipper domain attached at the C-terminus of the Fc C_{H}3 domains. Attachment of a leucine zipper is sufficient to cause preferential assembly of heterodimeric antibody heavy chains. See, e.g., Wranik et al (2012) J Biol Chem 287:43331-43339. As disclosed herein, one of a pair of Fc sequences attached to a leucine zipper-forming strand can be arbitrarily fused to the ActRIIA or ActRIIB polypeptide of the construct, with or without an optional linker, to generate a single-arm ActRIIA heteromultimer fusion construct or a single-arm ActRIIB heteromultimer fusion construct. This single chain can be coexpressed in a cell of choice along with the Fc sequence attached to a complementary leucine zipper-forming strand to favor generation of the desired multichain construct. Proteolytic digestion of the construct with the bacterial endoproteinase Lys-C post purification can release the leucine zipper domain, resulting in an Fc construct whose structure is identical to that of native Fc. In this example based on leucine zipper pairing, SEQ ID NO: 27 [hG1Fc-Ap1 (acidic)] and SEQ ID NO: 28 [hG1Fc-Bp1 (basic)] are examples of complementary IgG Fc sequences in which the engineered complimentary leucine zipper sequences are underlined, and the ActRIIA or ActRIIB polypeptide of the construct can be fused to either SEQ ID NO: 27 or SEQ ID NO: 28, but not both. Given the high degree of amino acid sequence identity between native hG1Fc, native hG2Fc, native hG3Fc, and native hG4Fc, it can be appreciated that leucine zipper-forming sequences attached, with or without an optional linker, to hG1Fc, hG2Fc, hG3Fc, or hG4Fc (see FIG. 4) will generate an Fc monomer which may be used in the complementary leucine zipper-forming pair below (SEQ ID NOs: 27 and 28).

1 THTCPPCPAP ELLGGPSVFL FPPKPKDTLM ISRTPEVTCV WDVSHEDPE
51 VKFNWYVDGV EVHNAKTKPR EEQYNSTYRV VSVLTVLHQD WLNGKEYKCK
101 VSNKALPAPI EKTISKAKGQ PREPQVYTLP PSREEMTKNQ VSLTCLVKGF
151 YPSDIAVEWE SNGQPENNYK TTPPVLDSDG SFFLYSKLTV DKSRWQQGNV
201 FSCSVMHEAL HNHYTQKSLS LSPGKGGSAQ LEKELQALEK ENAQLEWELQ
251 ALEKELAQGA T (SEQ ID NO: 27)
1 THTCPPCPAP ELLGGPSVFL FPPKPKDTLM ISRTPEVTCV WDVSHEDPE
51 VKFNWYVDGV EVHNAKTKPR EEQYNSTYRV VSVLTVLHQD WLNGKEYKCK
101 VSNKALPAPI EKTISKAKGQ PREPQVYTLP PSREEMTKNQ VSLTCLVKGF
151 YPSDIAVEWE SNGQPENNYK TTPPVLDSDG SFFLYSKLTV DKSRWQQGNV
201 FSCSVMHEAL HNHYTQKSLS LSPGKGGSAQ LKKKLQALKK KNAQLKWKLQ
251 ALKKKLAQGA T (SEQ ID NO: 28)

It is understood that different elements of the fusion proteins (e.g., immunoglobulin Fc fusion proteins) may be arranged in any manner that is consistent with desired functionality. For example, an ActRIIA or ActRIIB polypeptide domain may be placed C-terminal to a heterologous domain, or alternatively, a heterologous domain may be placed C-terminal to an ActRIIA or ActRIIB polypeptide domain. The ActRIIA or ActRIIB polypeptide domain and the heterologous domain need not be adjacent in a fusion protein, and additional domains or amino acid sequences may be included C- or N-terminal to either domain or between the domains. For example, a single-arm ActRIIA heteromultimer fusion construct or single-arm ActRIIB heteromultimer fusion construct may comprise an amino acid sequence as set forth in the formula A-B-C. The B portion corresponds to an ActRIIA or ActRIIB polypeptide domain. The A and C portions may be independently zero, one, or more than one amino acid, and both the A and C portions when present are heterologous to B. The A and/or C portions may be attached to the B portion via a linker sequence. In certain embodiments, an ActRIIA or ActRIIB fusion polypeptide comprises an amino acid sequence as set forth in the formula A-B-C, wherein A is a leader (signal) sequence, B consists of an ActRIIA or ActRIIB polypeptide domain, and C is a polypeptide portion that enhances one or more of in vivo stability, in vivo half-life, uptake/administration, tissue localization or distribution, formation of protein complexes, and/or purification. In certain embodiments, an ActRIIA or ActRIIB fusion polypeptide comprises an amino acid sequence as set forth in the formula A-B-C, wherein A is a TPA leader sequence, B consists of an ActRIIA or ActRIIB polypeptide domain, and C is an immunoglobulin Fc domain. Preferred fusion polypeptides comprise the amino acid sequence set forth in any one of SEQ ID NOs: 46, 48, 55, 57, 58, 59, 60, and 61.

In some embodiments, single-arm ActRIIA heteromultimers or single-arm ActRIIB heteromultimers of the present disclosure further comprise one or more heterologous portions (domains) so as to confer a desired property. For example, some fusion domains are particularly useful for isolation of the fusion proteins by affinity chromatography. Well-known examples of such fusion domains include, but are not limited to, polyhistidine, Glu-Glu, glutathione S-transferase (GST), thioredoxin, protein A, protein G, an immunoglobulin heavy-chain constant region (Fc), maltose binding protein (MBP), or human serum albumin. For the purpose of affinity purification, relevant matrices for affinity chromatography, such as glutathione-, amylase-, and nickel- or cobalt- conjugated resins are used. Many of such matrices are available in “kit” form, such as the Pharmacia GST purification system and the QIAexpress™ system (Qiagen) useful with (HIS₆) fusion partners. As another example, a fusion domain may be selected so as to facilitate detection of the ligand trap polypeptides. Examples of such detection domains include the various fluorescent proteins (e.g., GFP) as well as “epitope tags,” which are usually short peptide sequences for which a specific antibody is available. Well-known epitope tags for which specific monoclonal antibodies are readily available include FLAG, influenza virus haemagglutinin (HA), and c-myc tags. In some cases, the fusion domains have a protease cleavage site, such as for factor Xa or thrombin, which allows the relevant protease to partially digest the fusion proteins and thereby liberate the recombinant proteins therefrom. The liberated proteins can then be isolated from the fusion domain by subsequent chromatographic separation.

In certain embodiments, ActRIIA or ActRIIB polypeptides of the present disclosure comprise one or more modifications that are capable of stabilizing the polypeptides. For example, such modifications enhance the in vitro half-life of the polypeptides, enhance circulatory half-life of the polypeptides, and/or reduce proteolytic degradation of the polypeptides. Such stabilizing modifications include, but are not limited to, fusion polypeptides (including, for example, fusion polypeptides comprising an ActRIIA or ActRIIB polypeptide domain and a stabilizer domain), modifications of a glycosylation site (including, for example, addition of a glycosylation site to a polypeptide of the disclosure), and modifications of carbohydrate moiety (including, for example, removal of carbohydrate moieties from a polypeptide of the disclosure). As used herein, the term “stabilizer domain” not only refers to a fusion domain (e.g., an immunoglobulin Fc domain) as in the case of fusion polypeptides, but also includes nonproteinaceous modifications such as a carbohydrate moiety, or nonproteinaceous moiety, such as polyethylene glycol.

In preferred embodiments, single-arm ActRIIA heteromultimers or single-arm ActRIIB heteromultimers to be used in accordance with the methods described herein are isolated heteromultimers. As used herein, an isolated protein (e.g., heteromultimer) or polypeptide (e.g., heteromultimer) is one which has been separated from a component of its natural environment. In some embodiments, a single-arm heteromultimer of the disclosure is purified to greater than 95%, 96%, 97%, 98%, or 99% purity as determined by, for example, electrophoretic (e.g., SDS-PAGE, isoelectric focusing (IEF), capillary electrophoresis) or chromatographic (e.g., ion exchange or reverse phase HPLC). Methods for assessment of antibody purity are well known in the art [See, e.g., Flatman et al., (2007) J. Chromatogr. B 848:79-87].

In certain embodiments, ActRIIA or ActRIIB polypeptides, as well as single-arm heteromultimers thereof, of the disclosure, can be produced by a variety of art-known techniques. For example, polypeptides of the disclosure can be synthesized using standard protein chemistry techniques such as those described in Bodansky, M. Principles of Peptide Synthesis, Springer Verlag, Berlin (1993) and Grant G. A. (ed.), Synthetic Peptides: A User’s Guide, W. H. Freeman and Company, New York (1992). In addition, automated peptide synthesizers are commercially available (see, e.g., Advanced ChemTech Model 396; Milligen/Biosearch 9600). Alternatively, the polypeptides and complexes of the disclosure, including fragments or variants thereof, may be recombinantly produced using various expression systems [e.g., E. coli, Chinese Hamster Ovary (CHO) cells, COS cells, baculovirus] as is well known in the art. In a further embodiment, the modified or unmodified polypeptides of the disclosure may be produced by digestion of recombinantly produced full-length ActRIIA or ActRIIB polypeptides by using, for example, a protease, e.g., trypsin, thermolysin, chymotrypsin, pepsin, or paired basic amino acid converting enzyme (PACE). Computer analysis (using a commercially available software, e.g., MacVector, Omega, PCGene, Molecular Simulation, Inc.) can be used to identify proteolytic cleavage sites.

In some embodiments, a single-arm ActRIIB heteromultimer of the present disclosure comprises a first polypeptide covalently or non-covalently associated with a second polypeptide, wherein the first polypeptide comprises the amino acid sequence of a first member of an interaction pair and the amino acid sequence of ActRIIB; and the second polypeptide comprises the amino acid sequence of a second member of the interaction pair, and wherein the second polypeptide does not comprise ActRIIB.

In some embodiments, the single-arm ActRIIB heteromultimer comprises, consists, or consists essentially of an amino acid sequence that is at least 70%, 80%, 85%, 90%, 91%, 92%, 93%, 94% 95%, 96%, 97%, 98%, 99% or 100% identical to the sequence of any of SEQ ID NOs: 1, 2, 3, 4, 5, and 6; or at least 70%, 80%, 85%, 90%, 91%, 92%, 93%, 94% 95%, 96%, 97%, 98%, 99% or 100% identical to a polypeptide that begins at any one of amino acids 20, 21, 22, 23, 24, 25, 26, 27, 28, or 29 of SEQ ID NO: 1, and ends at any one of amino acids 109, 110, 111, 112, 113, 114, 115, 116, 117, 118, 119, 120, 121, 122, 123, 124, 125, 126, 127, 128, 129, 130, 131, 132, 133, or 134 of SEQ ID NO: 1.

In some embodiments, the single-arm ActRIIB heteromultimer comprises, consists, or consists essentially of an amino acid sequence that is at least 70%, 80%, 85%, 90%, 91%, 92%, 93%, 94% 95%, 96%, 97%, 98%, 99% or 100% identical to the sequence of SEQ ID NO: 1. In some embodiments, the single-arm ActRIIB heteromultimer comprises, consists, or consists essentially of an amino acid sequence that is at least 70%, 80%, 85%, 90%, 91%, 92%, 93%, 94% 95%, 96%, 97%, 98%, 99% or 100% identical to the sequence of SEQ ID NO: 2. In some embodiments, the single-arm ActRIIB heteromultimer comprises, consists, or consists essentially of an amino acid sequence that is at least 70%, 80%, 85%, 90%, 91%, 92%, 93%, 94% 95%, 96%, 97%, 98%, 99% or 100% identical to the sequence of SEQ ID NO: 3. In some embodiments, the single-arm ActRIIB heteromultimer comprises, consists, or consists essentially of an amino acid sequence that is at least 70%, 80%, 85%, 90%, 91%, 92%, 93%, 94% 95%, 96%, 97%, 98%, 99% or 100% identical to the sequence of SEQ ID NO: 4. In some embodiments, the single-arm ActRIIB heteromultimer comprises, consists, or consists essentially of an amino acid sequence that is at least 70%, 80%, 85%, 90%, 91%, 92%, 93%, 94% 95%, 96%, 97%, 98%, 99% or 100% identical to the sequence of SEQ ID NO: 5. In some embodiments, the single-arm ActRIIB heteromultimer comprises, consists, or consists essentially of an amino acid sequence that is at least 70%, 80%, 85%, 90%, 91%, 92%, 93%, 94% 95%, 96%, 97%, 98%, 99% or 100% identical to the sequence of SEQ ID NO: 6.

In some embodiments, the single-arm ActRIIB heteromultimer comprises, consists, or consists essentially of an amino acid sequence that is at least 70%, 80%, 85%, 90%, 91%, 92%, 93%, 94% 95%, 96%, 97%, 98%, 99% or 100% identical to a polypeptide that begins at any one of amino acids 20, 21, 22, 23, 24, 25, 26, 27, 28, or 29 of SEQ ID NO: 1, and ends at any one of amino acids 109, 110, 111, 112, 113, 114, 115, 116, 117, 118, 119, 120, 121, 122, 123, 124, 125, 126, 127, 128, 129, 130, 131, 132, 133, or 134 of SEQ ID NO: 1

In some embodiments, the single-arm ActRIIB heteromultimer does not comprise an acidic amino acid at the position corresponding to L79 of SEQ ID NO: 1. In some embodiments, the single-arm ActRIIB heteromultimer does not comprise an aspartic acid (D) at the position corresponding to L79 of SEQ ID NO: 1.

In some embodiments, the single-arm ActRIIB heteromultimer comprises the amino acid sequence of SEQ ID NO: 2. In some embodiments, the single-arm ActRIIB heteromultimer consists of the amino acid sequence of SEQ ID NO: 2. In some embodiments, the single-arm ActRIIB heteromultimer consists essentially of the amino acid sequence of SEQ ID NO: 2.

In some embodiments, the single-arm ActRIIB heteromultimer comprises the amino acid sequence of SEQ ID NO: 3. In some embodiments, the single-arm ActRIIB heteromultimer consists of the amino acid sequence of SEQ ID NO: 3. In some embodiments, the single-arm ActRIIB heteromultimer consists essentially of the amino acid sequence of SEQ ID NO: 3.

In some embodiments, the single-arm ActRIIB heteromultimer comprises the amino acid sequence of SEQ ID NO: 5. In some embodiments, the single-arm ActRIIB heteromultimer consists of the amino acid sequence of SEQ ID NO: 5. In some embodiments, the single-arm ActRIIB heteromultimer consists essentially of the amino acid sequence of SEQ ID NO: 5.

In some embodiments, the single-arm ActRIIB heteromultimer comprises the amino acid sequence of SEQ ID NO: 6. In some embodiments, the single-arm ActRIIB heteromultimer consists of the amino acid sequence of SEQ ID NO: 6. In some embodiments, the single-arm ActRIIB heteromultimer consists essentially of the amino acid sequence of SEQ ID NO: 6.

In some embodiments, the single-arm ActRIIB heteromultimer comprises the amino acid sequence of SEQ ID NO: 48. In some embodiments, the single-arm ActRIIB heteromultimer consists of the amino acid sequence of SEQ ID NO: 48. In some embodiments, the single-arm ActRIIB heteromultimer consists essentially of the amino acid sequence of SEQ ID NO: 48.

In some embodiments, the single-arm ActRIIB heteromultimer comprises the amino acid sequence of SEQ ID NO: 61. In some embodiments, the single-arm ActRIIB heteromultimer consists of the amino acid sequence of SEQ ID NO: 61. In some embodiments, the single-arm ActRIIB heteromultimer consists essentially of the amino acid sequence of SEQ ID NO: 61.

In some embodiments, a single-arm ActRIIA heteromultimer of the present disclosure comprises a first polypeptide covalently or non-covalently associated with a second polypeptide, wherein the first polypeptide comprises the amino acid sequence of a first member of an interaction pair and the amino acid sequence of ActRIIA; and the second polypeptide comprises the amino acid sequence of a second member of the interaction pair, and wherein the second polypeptide does not comprise ActRIIA.

In some embodiments, the single-arm ActRIIA heteromultimer comprises, consists, or consists essentially of an amino acid sequence that is at least 70%, 80%, 85%, 90%, 91%, 92%, 93%, 94% 95%, 96%, 97%, 98%, 99% or 100% identical to the sequence of any of SEQ ID Nos: 9, 10, and 11; or at least 70%, 80%, 85%, 90%, 91%, 92%, 93%, 94% 95%, 96%, 97%, 98%, 99% or 100% identical to a polypeptide that begins at any one of amino acids 21, 22, 23, 24, 25, 26, 27, 28, 29, or 30 of SEQ ID NO: 9, and ends at any one of amino acids 110, 111, 112, 113, 114, 115, 116, 117, 118, 119, 120, 121, 122, 123, 124, 125, 126, 127, 128, 129, 130, 131, 132, 133, 134 or 135 of SEQ ID NO: 9.

In some embodiments, the single-arm ActRIIA heteromultimer comprises, consists, or consists essentially of an amino acid sequence that is at least 70%, 80%, 85%, 90%, 91%, 92%, 93%, 94% 95%, 96%, 97%, 98%, 99% or 100% identical to the sequence of SEQ ID NO: 9. In some embodiments, the single-arm ActRIIA heteromultimer comprises, consists, or consists essentially of an amino acid sequence that is at least 70%, 80%, 85%, 90%, 91%, 92%, 93%, 94% 95%, 96%, 97%, 98%, 99% or 100% identical to the sequence of SEQ ID NO: 10. In some embodiments, the single-arm ActRIIA heteromultimer comprises, consists, or consists essentially of an amino acid sequence that is at least 70%, 80%, 85%, 90%, 91%, 92%, 93%, 94% 95%, 96%, 97%, 98%, 99% or 100% identical to the sequence of SEQ ID NO: 11.

In some embodiments, the single-arm ActRIIA heteromultimer comprises, consists, or consists essentially of an amino acid sequence that is at least 70%, 80%, 85%, 90%, 91%, 92%, 93%, 94% 95%, 96%, 97%, 98%, 99% or 100% identical to a polypeptide that begins at any one of amino acids 21, 22, 23, 24, 25, 26, 27, 28, 29, or 30 of SEQ ID NO: 9, and ends at any one of amino acids 110, 111, 112, 113, 114, 115, 116, 117, 118, 119, 120, 121, 122, 123, 124, 125, 126, 127, 128, 129, 130, 131, 132, 133, 134 or 135 of SEQ ID NO: 9.

In some embodiments, the single-arm ActRIIA heteromultimer comprises the amino acid sequence of SEQ ID NO: 10. In some embodiments, the single-arm ActRIIA heteromultimer consists of the amino acid sequence of SEQ ID NO: 10. In some embodiments, the single-arm ActRIIA heteromultimer consists essentially of the amino acid sequence of SEQ ID NO: 10.

In some embodiments, the single-arm ActRIIA heteromultimer comprises the amino acid sequence of SEQ ID NO: 11. In some embodiments, the single-arm ActRIIA heteromultimer consists of the amino acid sequence of SEQ ID NO: 11. In some embodiments, the single-arm ActRIIA heteromultimer consists essentially of the amino acid sequence of SEQ ID NO: 11.

In some embodiments, the single-arm ActRIIA heteromultimer comprises the amino acid sequence of SEQ ID NO: 57. In some embodiments, the single-arm ActRIIA heteromultimer consists of the amino acid sequence of SEQ ID NO: 57. In some embodiments, the single-arm ActRIIA heteromultimer consists essentially of the amino acid sequence of SEQ ID NO: 57.

In some embodiments, the single-arm ActRIIA heteromultimer comprises the amino acid sequence of SEQ ID NO: 59. In some embodiments, the single-arm ActRIIA heteromultimer consists of the amino acid sequence of SEQ ID NO: 59. In some embodiments, the single-arm ActRIIA heteromultimer consists essentially of the amino acid sequence of SEQ ID NO: 59.

In some embodiments, the single-arm heteromultimer is a heterodimer. In some embodiments, the first member of an interaction pair comprises a first constant region from an IgG heavy chain. In some embodiments, the first constant region from an IgG heavy chain is a first immunoglobulin Fc domain. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence selected from any one of SEQ ID NOs: 14-28.

In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 14. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 15. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 16. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 17. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 18. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 19. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 20. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 21. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 22. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 23. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 24. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 25. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 26. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 27. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 28.

In some embodiments, the second member of an interaction pair comprises a second constant region from an IgG heavy chain. In some embodiments, the second constant region from an IgG heavy chain is a first immunoglobulin Fc domain. In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence selected from any one of SEQ ID NOs: 14-28.

In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 14. In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 15. In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 16. In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 17. In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 18. In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 19. In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 20. In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 21. In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 22. In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 23. In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 24. In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 25. In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 26. In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 27. In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 28.

In some embodiments, the first polypeptide comprises an amino acid sequence that is at least 70%, 80%, 85%, 90%, 91%, 92%, 93%, 94% 95%, 96%, 97%, 98%, 99% or 100% identical to a sequence selected from any one of SEQ ID NOs: 46, 48, 55, 57, 58, 59, 60, and 61.

In some embodiments, the first polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 46. In some embodiments, the first polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 48. In some embodiments, the first polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 55. In some embodiments, the first polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 57. In some embodiments, the first polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 58. In some embodiments, the first polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 59. In some embodiments, the first polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 60. In some embodiments, the first polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 61.

In some embodiments, the second polypeptide comprises an amino acid sequence that is at least 70%, 80%, 85%, 90%, 91%, 92%, 93%, 94% 95%, 96%, 97%, 98%, 99% or 100% identical to a sequence selected from any one of SEQ ID NOs: 49, 51, 62, and 63.

In some embodiments, the second polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 49. In some embodiments, the second polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 51.. In some embodiments, the second polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 62. In some embodiments, the second polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 63.

In some embodiments, a heteromultimer complex binds to one or more of ActRIIA or ActRIIB ligands selected from the group consisting of activin A, activin B, GDF11, GDF8, GDF3, BMP5, BMP6, and BMP10. In some embodiments, a single-arm ActRIIB heteromultimer binds to activin B and GDF11. In some embodiments, a single-arm ActRIIB heteromultimer binds to GDF8 and activin A. In some embodiments, a single-arm ActRIIA heteromultimer binds to activin A over activin B and GDF11. In some embodiments, a single-arm ActRIIA heteromultimer binds to GDF8.

### 3. Linkers

The disclosure provides for single-arm ActRIIA heteromultimers or single-arm ActRIIB heteromultimers, and in these embodiments, the ActRIIA or ActRIIB polypeptide and a first member of an interaction pair (e.g., a constant region from an IgG heavy chain) may be connected by means of a linker. In some embodiments, a single-arm ActRIIA heteromultimer comprises a linker domain positioned between the ActRIIA polypeptide and the first member of an interaction pair. In some embodiments, a single-arm ActRIIB heteromultimer comprises a linker domain positioned between the ActRIIB polypeptide and the first member of an interaction pair. In some embodiments, the linkers are glycine and serine rich linkers. Other near neutral amino acids, such as, but not limited to, Thr, Asn, Pro and Ala, may also be used in the linker sequence. In some embodiments, the linker comprises various permutations of amino acid sequences containing Gly and Ser. In some embodiments, the linker is greater than 10 amino acids in length. In further embodiments, the linkers have a length of at least 12, 15, 20, 21, 25, 30, 35, 40, 45 or 50 amino acids. In some embodiments, the linker is less than 40, 35, 30, 25, 22 or 20 amino acids. In some embodiments, the linker is 10-50, 10-40, 10-30, 10-25, 10-21, 10-15, 10, 15-25, 17-22, 20, or 21 amino acids in length. In preferred embodiments, the linker comprises the amino acid sequence GlyGlyGlyGlySer (GGGGS) (SEQ ID NO: 29), or repetitions thereof (GGGGS)n, where n ≥ 2 (SEQ ID NO: 30). In particular embodiments n ≥ 3, or n = 3-10. In preferred embodiments, n ≥ 4, or n = 4-10. In some embodiments, n is not greater than 4 in a (GGGGS)n linker (SEQ ID NO: 29). In some embodiments, n = 4-10, 4-9, 4-8, 4-7, 4-6, 4-5, 5-8, 5-7, or 5-6. In some embodiments, n = 3, 4, 5, 6, or 7. In particular embodiments, n = 4. In some embodiments, a linker comprising a (GGGGS)n sequence (SEQ ID NO: 29) also comprises an N-terminal threonine. In some embodiments, the linker is any one of the following:

GGG (SEQ ID NO: 31)
GGGG (SEQ ID NO: 32)
GGGGSGGGGS (SEQ ID NO: 33)
SGGG (SEQ ID NO: 34)
SGGGG (SEQ ID NO: 35)
TGGG (SEQ ID NO: 36)
TGGGG (SEQ ID NO: 37)
TGGGGSGGGGS (SEQ ID NO: 38)
TGGGGSGGGGSGGGGS (SEQ ID NO: 39)
TGGGGSGGGGSGGGGSGGGGS (SEQ ID NO: 40)
TGGGGSGGGGSGGGGSGGGGSGGGGS (SEQ ID NO: 41)
TGGGGSGGGGSGGGGSGGGGSGGGGSGGGGS (SEQ ID NO: 42) or
TGGGGSGGGGSGGGGSGGGGSGGGGSGGGGSGGGGS (SEQ ID NO: 43).

In some embodiments, the linker comprises the amino acid sequence of TGGGPKSCDK (SEQ ID NO: 44). In some embodiments, the linker is any one of SEQ ID NOs: 31-44) lacking the N-terminal threonine. In some embodiments, the linker does not comprise the amino acid sequence of SEQ ID NO: 42 or 43. In some embodiments, the linker comprises an amino acid sequence selected from any one of SEQ ID NOs: 29-44.

### 4. Nucleic Acids Encoding ActRIIA or ActRIIB Polypeptides

In certain embodiments, the present disclosure provides isolated and/or recombinant nucleic acids encoding ActRIIA or ActRIIB polypeptides (including fragments, functional variants, and fusion proteins thereof) disclosed herein. For example, SEQ ID NO: 12 encodes the naturally occurring human ActRIIA precursor polypeptide, while SEQ ID NO: 13 encodes the processed extracellular domain of ActRIIA. The subject nucleic acids may be single-stranded or double stranded. Such nucleic acids may be DNA or RNA molecules. These nucleic acids may be used, for example, in methods for making ActRIIA or ActRIIB single-arm heteromultimers of the present disclosure.

As used herein, isolated nucleic acid(s) refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

In certain embodiments, nucleic acids encoding ActRIIA or ActRIIB polypeptides of the present disclosure are understood to include nucleic acids that are variants of any one of SEQ ID NOs: 7, 8, 12, and 13. In certain embodiments, nucleic acid encoding a first and/or second member of an interaction pair (e.g., a constant region from an IgG heavy chain) are understood to include nucleic acids that are variants of any one of SEQ ID NOs: 50. In some embodiments, nucleic acids encoding single-arm ActRIIA heteromultimer fusions or single-arm ActRIIB heteromultimer Fc fusions of the present disclosure are understood to include nucleic acids that are variants of any one of SEQ ID NOs: 47 and 56. In some embodiments, a single-arm ActRIIA heteromultimer fusion comprises a single-arm ActRIIA heterodimer Fc fusion, comprising the amino acid sequence of SEQ ID NO: 56. In some embodiments, a single-arm ActRIIB heteromultimer fusion comprises a single-arm ActRIIB heterodimer Fc fusion, comprising the amino acid sequence of SEQ ID NO: 47. Variant nucleotide sequences include sequences that differ by one or more nucleotide substitutions, additions, or deletions including allelic variants, and therefore, will include coding sequences that differ from the nucleotide sequence designated in any one of SEQ ID NOs: 7, 8, 12, 13, 47, 50, and 56.In certain embodiments, ActRIIA or ActRIIB polypeptides of the present disclosure are encoded by isolated or recombinant nucleic acid sequences that are at least 80%, 85%, 90%, 91%, 92%, 93%, 94%, 95%, 96%, 97%, 98%, or 99% identical to SEQ ID NOs: 7, 8, 12, 13. In certain embodiments, a first and/or second member of an interaction pair (e.g., a constant region from an IgG heavy chain) of the present disclosure are encoded by isolated or recombinant nucleic acid sequences that are at least 80%, 85%, 90%, 91%, 92%, 93%, 94%, 95%, 96%, 97%, 98%, or 99% identical to SEQ ID NOs: 50. In some embodiments, a single-arm ActRIIA heteromultimer fusion of the present disclosure are encoded by isolated or recombinant nucleic acid sequences that are at least 80%, 85%, 90%, 91%, 92%, 93%, 94%, 95%, 96%, 97%, 98%, or 99% identical to SEQ ID NO: 56. In some embodiments, a single-arm ActRIIB heteromultimer fusion of the present disclosure are encoded by isolated or recombinant nucleic acid sequences that are at least 80%, 85%, 90%, 91%, 92%, 93%, 94%, 95%, 96%, 97%, 98%, or 99% identical to SEQ ID NO: 47. One of ordinary skill in the art will appreciate that nucleic acid sequences that are at least 80%, 85%, 90%, 91%, 92%, 93%, 94%, 95%, 96%, 97%, 98%, or 99% identical to the sequences complementary to SEQ ID NOs: 7, 8, 12, 13, 47, 50, and 56 are also within the scope of the present disclosure. In further embodiments, the nucleic acid sequences of the disclosure can be isolated, recombinant, and/or fused with a heterologous nucleotide sequence or in a DNA library.

In other embodiments, nucleic acids of the present disclosure also include nucleotide sequences that hybridize under highly stringent conditions to the nucleotide sequence designated in SEQ ID NOs: 7, 8, 12, 13, 47, 50, and 56, the complement sequence of SEQ ID NOs: 7, 8, 12, 13, 47, 50, and 56, or fragments thereof. One of ordinary skill in the art will understand readily that appropriate stringency conditions which promote DNA hybridization can be varied. For example, one could perform the hybridization at 6.0 x sodium chloride/sodium citrate (SSC) at about 45° C., followed by a wash of 2.0 x SSC at 50° C. For example, the salt concentration in the wash step can be selected from a low stringency of about 2.0 x SSC at 50° C. to a high stringency of about 0.2 x SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6 x SSC at room temperature followed by a wash at 2 x SSC at room temperature.

Isolated nucleic acids which differ from the nucleic acids as set forth in SEQ ID NOs: 7, 8, 12, 13, 47, 50, and 56 due to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this disclosure.

In certain embodiments, the recombinant nucleic acids of the present disclosure may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In some embodiments, the expression vector contains a selectable marker gene to allow the selection of transformed host cells. Selectable marker genes are well known in the art and will vary with the host cell used.

In certain aspects of the present disclosure, the subject nucleic acid is provided in an expression vector comprising a nucleotide sequence encoding an ActRIIA or ActRIIB polypeptide, a first and/or second member of an interaction pair (e.g., a constant region from an IgG heavy chain), and/or a single-arm ActRIIA heteromultimer or single-arm ActRIIB heteromultimer, and operably linked to at least one regulatory sequence. Regulatory sequences are art-recognized and are selected to direct expression of the ActRIIA or ActRIIB polypeptide, a first and/or second member of an interaction pair (e.g., a constant region from an IgG heavy chain), and/or a single-arm ActRIIA heteromultimer or single-arm ActRIIB heteromultimer. Accordingly, the term regulatory sequence includes promoters, enhancers, and other expression control elements. Exemplary regulatory sequences are described in Goeddel; Gene Expression Technology: Methods in Enzymology, Academic Press, San Diego, CA (1990). For instance, any of a wide variety of expression control sequences that control the expression of a DNA sequence when operatively linked to it may be used in these vectors to express DNA sequences encoding an ActRIIA or ActRIIB polypeptide, a first and/or second member of an interaction pair (e.g., a constant region from an IgG heavy chain), and/or a single-arm ActRIIA heteromultimer or single-arm ActRIIB heteromultimer. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector’s copy number, the ability to control that copy number and the expression of any other protein encoded by the vector, such as antibiotic markers, should also be considered.

A recombinant nucleic acid of the present disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant ActRIIA or ActRIIB polypeptide, a first and/or second member of an interaction pair (e.g., a constant region from an IgG heavy chain), and/or a single-arm ActRIIA heteromultimer or single-arm ActRIIB heteromultimer, include plasmids and other vectors. For instance, suitable vectors include plasmids of the following types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, see, e.g., Molecular Cloning A Laboratory Manual, 3rd Ed., ed. by Sambrook, Fritsch and Maniatis (Cold Spring Harbor Laboratory Press, 2001). In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the ß-gal containing pBlueBac III).

In a preferred embodiment, a vector will be designed for production of the subject ActRIIA or ActRIIB polypeptide, a first and/or second member of an interaction pair (e.g., a constant region from an IgG heavy chain), and/or a single-arm ActRIIA heteromultimer or single-arm ActRIIB heteromultimer in CHO cells, such as a Pcmv-Script vector (Stratagene, La Jolla, Calif.), pcDNA4 vectors (Invitrogen, Carlsbad, Calif.) and pCI-neo vectors (Promega, Madison, Wisc.). As will be apparent, the subject gene constructs can be used to cause expression of the subject ActRIIA or ActRIIB polypeptide , a first and/or second member of an interaction pair (e.g., a constant region from an IgG heavy chain), and/or a single-arm ActRIIA heteromultimer or single-arm ActRIIB heteromultimer in cells propagated in culture, e.g., to produce proteins, including fusion proteins or variant proteins, for purification.

This disclosure also pertains to a host cell transfected with a recombinant gene including a coding sequence for one or more of the subject ActRIIA or ActRIIB polypeptides, a first and/or second member of an interaction pair (e.g., a constant region from an IgG heavy chain), and/or a single-arm ActRIIA heteromultimer or single-arm ActRIIB heteromultimer. The host cell may be any prokaryotic or eukaryotic cell. For example, an ActRIIA or ActRIIB polypeptide , a first and/or second member of an interaction pair (e.g., a constant region from an IgG heavy chain), and/or a single-arm ActRIIA heteromultimer or single-arm ActRIIB heteromultimer of the disclosure may be expressed in bacterial cells such as E. coli, insect cells (e.g., using a baculovirus expression system), yeast, or mammalian cells [e.g. a Chinese hamster ovary (CHO) cell line]. Other suitable host cells are known to those skilled in the art.

Accordingly, the present disclosure further pertains to methods of producing the subject ActRIIA or ActRIIB polypeptides, a first and/or second member of an interaction pair (e.g., a constant region from an IgG heavy chain), and/or a single-arm ActRIIA heteromultimer or single-arm ActRIIB heteromultimer. For example, a host cell transfected with an expression vector encoding an ActRIIA or ActRIIB polypeptide, a first and/or second member of an interaction pair (e.g., a constant region from an IgG heavy chain), and/or a single-arm ActRIIA heteromultimer or single-arm ActRIIB heteromultimer can be cultured under appropriate conditions to allow expression of the ActRIIA or ActRIIB polypeptide, a first and/or second member of an interaction pair (e.g., a constant region from an IgG heavy chain), and/or a single-arm ActRIIA heteromultimer or single-arm ActRIIB heteromultimer to occur. The polypeptide(s) may be secreted and isolated from a mixture of cells and medium containing the polypeptide(s). Alternatively, the ActRIIA or ActRIIB polypeptide, a first and/or second member of an interaction pair (e.g., a constant region from an IgG heavy chain), and/or a single-arm ActRIIA heteromultimer or single-arm ActRIIB heteromultimer may be isolated from a cytoplasmic or membrane fraction obtained from harvested and lysed cells. A cell culture includes host cells, media and other byproducts. Suitable media for cell culture are well known in the art. The subject polypeptides can be isolated from cell culture medium, host cells, or both, using techniques known in the art for purifying proteins, including ion-exchange chromatography, gel filtration chromatography, ultrafiltration, electrophoresis, immunoaffinity purification with antibodies specific for particular epitopes of the ActRIIA or ActRIIB polypeptides, a first and/or second member of an interaction pair (e.g., a constant region from an IgG heavy chain), and/or a single-arm ActRIIA heteromultimer or single-arm ActRIIB heteromultimer and affinity purification with an agent that binds to a domain fused to ActRIIA or ActRIIB polypeptide, a first and/or second member of an interaction pair (e.g., a constant region from an IgG heavy chain), and/or a single-arm ActRIIA heteromultimer or single-arm ActRIIB heteromultimer (e.g., a protein A column may be used to purify any polypeptides disclosed herein). In some embodiments, ActRIIA or ActRIIB polypeptides, a first and/or second member of an interaction pair (e.g., a constant region from an IgG heavy chain), and/or a single-arm ActRIIA heteromultimer or single-arm ActRIIB heteromultimer comprise a domain which facilitates purification.

In some embodiments, purification is achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. A single-arm ActRIIA heteromultimer or single-arm, ActRIIB heteromultimer, for example, may be purified to a purity of >90%, >91%, >92%, >93%, >94%, >95%, >96%, >97%, >98%, or >99% as determined by size exclusion chromatography and >90%, >91%, >92%, >93%, >94%, >95%, >96%, >97%, >98%, or >99% as determined by SDS PAGE. The target level of purity should be one that is sufficient to achieve desirable results in mammalian systems, particularly non-human primates, rodents (mice), and humans.

In another embodiment, a fusion gene coding for a purification leader sequence, such as a poly-(His)/enterokinase cleavage site sequence at the N-terminus of the desired portion of the recombinant ActRIIA or ActRIIB polypeptide, a first and/or second member of an interaction pair (e.g., a constant region from an IgG heavy chain), and/or a single-arm ActRIIA heteromultimer or single-arm ActRIIB heteromultimer, can allow purification of the expressed construct by affinity chromatography using a Ni²⁺ metal resin. The purification leader sequence can then be subsequently removed by treatment with enterokinase to provide the purified ActRIIA or ActRIIB polypeptide or protein complex. See, e.g., Hochuli et al. (1987) J. Chromatography 411:177; and Janknecht et al. (1991) PNAS USA 88:8972.

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence. See, e.g., Current Protocols in Molecular Biology, eds. Ausubel et al., John Wiley & Sons: 1992.

### 5. Screening Assays

In certain aspects, the present disclosure relates to the use of single-arm ActRIIA heteromultimers or single-arm ActRIIB heteromultimers to identify compounds (agents) which are agonists or antagonists of ActRIIA or ActRIIB. Compounds identified through this screening can be tested to assess their ability to treat pulmonary diseases or conditions (e.g., pulmonary hypertension (PH), pulmonary arterial hypertension (PAH), idiopathic pulmonary fibrosis (IPF), interstitial lung disease (ILD)). These compounds can be tested, for example, in animal models.

In certain aspects, the present disclosure relates to the use of the subject single-arm ActRIIA heteromultimers or single-arm ActRIIB heteromultimers to identify compounds (agents) which may be used to treat, prevent, or reduce the progression rate and/or severity of pulmonary hypertension (PH), particularly treating, preventing or reducing the progression rate and/or severity of one or more PH-associated complications.

There are numerous approaches to screening for therapeutic agents for treating PH by targeting signaling (e.g., Smad signaling) of one or more ActRIIA or ActRIIB ligands. In certain embodiments, high-throughput screening of compounds can be carried out to identify agents that perturb ActRIIA or ActRIIB ligand-mediated effects on a selected cell line. In certain embodiments, the assay is carried out to screen and identify compounds that specifically inhibit or reduce binding of an ActRIIA or ActRIIB ligand (e.g., activin A, activin B, GDF11, GDF8, GDF3, BMP5, BMP6, or BMP10) to its binding partner, such as ActRIIA or ActRIIB. Alternatively, the assay can be used to identify compounds that enhance binding of an ActRIIA or ActRIIB ligand to its binding partner such as ActRIIA or ActRIIB. In a further embodiment, the compounds can be identified by their ability to interact with ActRIIA or ActRIIB.

A variety of assay formats will suffice, and, in light of the present disclosure, those not expressly described herein will nevertheless be comprehended by one of ordinary skill in the art. As described herein, the test compounds (agents) of the invention may be created by any combinatorial chemical method. Alternatively, the subject compounds may be naturally occurring biomolecules synthesized in vivo or in vitro. Compounds (agents) to be tested for their ability to act as modulators of tissue growth can be produced, for example, by bacteria, yeast, plants or other organisms (e.g., natural products), produced chemically (e.g., small molecules, including peptidomimetics), or produced recombinantly. Test compounds contemplated by the present invention include non-peptidyl organic molecules, peptides, polypeptides, peptidomimetics, sugars, hormones, and nucleic acid molecules. In certain embodiments, the test agent is a small organic molecule having a molecular weight of less than about 2,000 Daltons.

The test compounds of the disclosure can be provided as single, discrete entities, or provided in libraries of greater complexity, such as made by combinatorial chemistry. These libraries can comprise, for example, alcohols, alkyl halides, amines, amides, esters, aldehydes, ethers and other classes of organic compounds. Presentation of test compounds to the test system can be in either an isolated form or as mixtures of compounds, especially in initial screening steps. Optionally, the compounds may be optionally derivatized with other compounds and have derivatizing groups that facilitate isolation of the compounds. Nonlimiting examples of derivatizing groups include biotin, fluorescein, digoxygenin, green fluorescent protein, isotopes, polyhistidine, magnetic beads, glutathione S-transferase (GST), photoactivatable crosslinkers or any combinations thereof.

In many drug-screening programs which test libraries of compounds and natural extracts, high-throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity or bioavailability of the test compound can be generally ignored in the in vitro system, the assay instead being focused primarily on the effect of the drug on the molecular target as may be manifest in an alteration of binding affinity between an ActRIIA or ActRIIB ligand (e.g., activin A, activin B, GDF11, GDF8, GDF3, BMP5, BMP6, or BMP10) to its binding partner, such as ActRIIA or ActRIIB.

Merely to illustrate, in an exemplary screening assay of the present disclosure, the compound of interest is contacted with an isolated and purified ActRIIB polypeptide which is ordinarily capable of binding to an ActRIIB ligand, as appropriate for the intention of the assay. To the mixture of the compound and ActRIIB polypeptide is then added to a composition containing an ActRIIB ligand (e.g., GDF11). Detection and quantification of ActRIIB/ActRIIB-ligand complexes provides a means for determining the compound’s efficacy at inhibiting (or potentiating) complex formation between the ActRIIB polypeptide and its binding protein. The efficacy of the compound can be assessed by generating dose-response curves from data obtained using various concentrations of the test compound. Moreover, a control assay can also be performed to provide a baseline for comparison. For example, in a control assay, isolated and purified ActRIIB ligand is added to a composition comprising the ActRIIB polypeptide (e.g., a single-arm ActRIIB heteromultimer), and the formation of ActRIIB/ActRIIB ligand complex is quantitated in the absence of the test compound. It will be understood that, in general, the order in which the reactants may be admixed can be varied, and can be admixed simultaneously. Moreover, in place of purified proteins, cellular extracts and lysates may be used to render a suitable cell-free assay system.

Complex formation between an ActRIIA or ActRIIB ligand and its binding protein may be detected by a variety of techniques. For instance, modulation of the formation of complexes can be quantitated using, for example, detectably labeled proteins such as radiolabeled (e.g., ³²P, ³⁵S, ¹⁴C or ³H), fluorescently labeled (e.g., FITC), or enzymatically labeled ActRIIB polypeptide and/or its binding protein, by immunoassay, or by chromatographic detection.

In certain embodiments, the present disclosure contemplates the use of fluorescence polarization assays and fluorescence resonance energy transfer (FRET) assays in measuring, either directly or indirectly, the degree of interaction between an ActRIIA or ActRIIB ligand and its binding protein. Further, other modes of detection, such as those based on optical waveguides (see, e.g., PCT Publication WO 96/26432 and U.S. Pat. No. 5,677,196), surface plasmon resonance (SPR), surface charge sensors, and surface force sensors, are compatible with many embodiments of the disclosure.

Moreover, the present disclosure contemplates the use of an interaction trap assay, also known as the “two-hybrid assay,” for identifying agents that disrupt or potentiate interaction between an ActRIIA or ActRIIB ligand and its binding partner. See, e.g., U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696). In a specific embodiment, the present disclosure contemplates the use of reverse two-hybrid systems to identify compounds (e.g., small molecules or peptides) that dissociate interactions between an ActRIIA or ActRIIB ligand and its binding protein [see, e.g., Vidal and Legrain, (1999) Nucleic Acids Res 27:919-29; Vidal and Legrain, (1999) Trends Biotechnol 17:374-81; and U.S. Pat. Nos. 5,525,490; 5,955,280; and 5,965,368].

In certain embodiments, the subject compounds are identified by their ability to interact with an ActRIIA or ActRIIB ligand. The interaction between the compound and the ActRIIA or ActRIIB ligand may be covalent or non-covalent. For example, such interaction can be identified at the protein level using in vitro biochemical methods, including photocrosslinking, radiolabeled ligand binding, and affinity chromatography [see, e.g., Jakoby WB et al. (1974) Methods in Enzymology 46:1]. In certain cases, the compounds may be screened in a mechanism-based assay, such as an assay to detect compounds which bind to an ActRIIA or ActRIIB ligand. This may include a solid-phase or fluid-phase binding event. Alternatively, the gene encoding an ActRIIA or ActRIIB ligand can be transfected with a reporter system (e.g., β-galactosidase, luciferase, or green fluorescent protein) into a cell and screened against the library preferably by high-throughput screening or with individual members of the library. Other mechanism-based binding assays may be used; for example, binding assays which detect changes in free energy. Binding assays can be performed with the target fixed to a well, bead or chip or captured by an immobilized antibody or resolved by capillary electrophoresis. The bound compounds may be detected usually using colorimetric endpoints or fluorescence or surface plasmon resonance.

### 6. Therapeutic Uses

In part, the present disclosure relates to methods of treating pulmonary diseases or conditions (e.g., pulmonary hypertension (PH), pulmonary arterial hypertension (PAH), idiopathic pulmonary fibrosis (IPF), interstitial lung disease (ILD)), comprising administering to a patient in need thereof an effective amount of a single-arm ActRIIA heteromultimer or single-arm ActRIIB heteromultimer, or combinations of single-arm ActRIIA heteromultimers or single-arm ActRIIB heteromultimers, of the present disclosure, can be used to treat or prevent a disease or condition that is associated with abnormal activity of a ActRIIA or ActRIIB polypeptide, and/or an ActRIIA or ActRIIB ligand (e.g., Activin A, activin B, GDF11, GDF8, GDF3, BMP5, BMP6, and BMP10).

In certain embodiments, the present invention provides methods of treating or preventing an individual in need thereof through administering to the individual a therapeutically effective amount of a single-arm ActRIIA heteromultimer or single-arm ActRIIB heteromultimer, or combinations of single-arm ActRIIA heteromultimers or single-arm ActRIIB heteromultimers, as described herein, optionally in combination with one or more additional active agents and/or supportive therapies

The terms “treatment”, “treating”, “alleviation” and the like are used herein to generally mean obtaining a desired pharmacologic and/or physiologic effect, and may also be used to refer to improving, alleviating, and/or decreasing the severity of one or more clinical complication of a condition being treated. The effect may be prophylactic in terms of completely or partially delaying the onset or recurrence of a disease, condition, or complications thereof, and/or may be therapeutic in terms of a partial or complete cure for a disease or condition and/or adverse effect attributable to the disease or condition. “Treatment” as used herein covers any treatment of a disease or condition of a mammal, particularly a human. As used herein, a therapeutic that “prevents” a disorder or condition refers to a compound that, in a statistical sample, reduces the occurrence of the disorder or condition in a treated sample relative to an untreated control sample, or delays the onset of the disease or condition, relative to an untreated control sample.

In general, treatment or prevention of a disease or condition as described in the present disclosure is achieved by administering a single-arm ActRIIA heteromultimer or a single-arm ActRIIB heteromultimer in an effective amount. An effective amount of an agent refers to an amount effective, at dosages and for periods of time necessary, to achieve the desired therapeutic or prophylactic result. A therapeutically effective amount of an agent of the present disclosure may vary according to factors such as the disease state, age, sex, and weight of the individual, and the ability of the agent to elicit a desired response in the individual. A prophylactically effective amount refers to an amount effective, at dosages and for periods of time necessary, to achieve the desired prophylactic result.

The terms “patient”, “subject”, or “individual” are used interchangeably herein and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In particular embodiments, the patient, subject or individual is a human.

In certain aspects, the disclosure contemplates the use of a single-arm ActRIIA heteromultimer or a single-arm ActRIIB heteromultimer, in combination with one or more additional active agents or other supportive therapy for treating or preventing a disease or condition (e.g., pulmonary hypertension, pulmonary arterial hypertension, and ILD). As used herein, “in combination with”, “combinations of”, “combined with”, or “conjoint” administration refers to any form of administration such that additional active agents or supportive therapies (e.g., second, third, fourth, etc.) are still effective in the body (e.g., multiple compounds are simultaneously effective in the patient for some period of time, which may include synergistic effects of those compounds). Effectiveness may not correlate to measurable concentration of the agent in blood, serum, or plasma. For example, the different therapeutic compounds can be administered either in the same formulation or in separate formulations, either concomitantly or sequentially, and on different schedules. Thus, a subject who receives such treatment can benefit from a combined effect of different active agents or therapies. One or more a single-arm ActRIIA heteromultimer or a single-arm ActRIIB heteromultimers of the disclosure can be administered concurrently with, prior to, or subsequent to, one or more other additional agents or supportive therapies, such as those disclosed herein. In general, each active agent or therapy will be administered at a dose and/or on a time schedule determined for that particular agent. The particular combination to employ in a regimen will take into account compatibility of the a single-arm ActRIIA heteromultimer or a single-arm ActRIIB heteromultimer of the present disclosure with the additional active agent or therapy and/or the desired effect.

In some embodiments, the disclosure contemplates methods of treating one or more complications of pulmonary hypertension (e.g., smooth muscle and/or endothelial cell proliferation in the pulmonary artery, angiogenesis in the pulmonary artery, dyspnea, chest pain, pulmonary vascular remodeling, right ventricular hypertrophy, and pulmonary fibrosis) comprising administering to a subject in need thereof an effective amount of a single-arm ActRIIA heteromultimer or a single-arm ActRIIB heteromultimer. In some embodiments, the disclosure contemplates methods of preventing one or more complications of pulmonary hypertension comprising administering to a subject in need thereof an effective amount of a single-arm ActRIIA heteromultimer or a single-arm ActRIIB heteromultimer. In some embodiments, the disclosure contemplates methods of reducing the progression rate of pulmonary hypertension comprising administering to a subject in need thereof an effective amount of a single-arm ActRIIA heteromultimer or a single-arm ActRIIB heteromultimer. In some embodiments, the disclosure contemplates methods of reducing the progression rate of one or more complications of pulmonary hypertension comprising administering to a subject in need thereof an effective amount of a single-arm ActRIIA heteromultimer or a single-arm ActRIIB heteromultimer. In some embodiments, the disclosure contemplates methods of reducing the severity of pulmonary hypertension comprising administering to a subject in need thereof an effective amount of a single-arm ActRIIA heteromultimer or a single-arm ActRIIB heteromultimer. In some embodiments, the disclosure contemplates methods of reducing the severity of one or more complications of pulmonary hypertension comprising administering to a subject in need thereof an effective amount of a single-arm ActRIIA heteromultimer or a single-arm ActRIIB heteromultimer. In some embodiments, pulmonary hypertension is pulmonary arterial hypertension (PAH).

Optionally, methods disclosed herein for treating, preventing, or reducing the progression rate and/or severity of pulmonary hypertension, particularly treating, preventing, or reducing the progression rate and/or severity of one or more complications of pulmonary hypertension, may further comprise administering to the subject one or more additional active agents and/or supportive therapies for treating pulmonary hypertension. In some embodiments, a subject is administered an additional active agent and/or supportive therapy for treating pulmonary hypertension. For example, the subject also may be administered one or more supportive therapies or active agents selected from the group consisting of: prostacyclin and derivatives thereof (e.g., epoprostenol, treprostinil, and iloprost); prostacyclin receptor agonists (e.g., selexipag); endothelin receptor antagonists (e.g., thelin, ambrisentan, macitentan, and bosentan); calcium channel blockers (e.g., amlodipine, diltiazem, and nifedipine; anticoagulants (e.g., warfarin); diuretics; oxygen therapy; atrial septostomy; pulmonary thromboendarterectomy; phosphodiesterase type 5 inhibitors (e.g., sildenafil and tadalafil); activators of soluble guanylate cyclase (e.g., cinaciguat and riociguat); ASK-1 inhibitors (e.g., CIIA; SCH79797; GS-4997; MSC2032964A; 3H-naphtho[1,2,3-de]quiniline-2,7-diones, NQDI-1; 2-thioxo-thiazolidines, 5-bromo-3-(4-oxo-2-thioxo-thiazolidine-5-ylidene)-1,3-dihydro-indol-2-one); NF-κB antagonists (e.g., dh404, CDDO-epoxide; 2.2-difluoropropionamide; C28 imidazole (CDDO-Im); 2-cyano-3,12-dioxoolean-1,9-dien-28-oic acid (CDDO); 3-Acetyloleanolic Acid; 3-Triflouroacetyloleanolic Acid; 28-Methyl-3-acetyloleanane; 28-Methyl-3-trifluoroacetyloleanane; 28-Methyloxyoleanolic Acid; SZC014; SCZ015; SZC017; PEGylated derivatives of oleanolic acid; 3-O-(beta-D-glucopyranosyl) oleanolic acid; 3-O-[beta-D-glucopyranosyl-(1-->3)-beta-D-glucopyranosyl] oleanolic acid; 3-O-[beta-D-glucopyranosyl-(1-->2)-beta-D-glucopyranosyl] oleanolic acid; 3-O-[beta-D-glucopyranosyl-(1-->3)-beta-D-glucopyranosyl] oleanolic acid 28-O-beta-D-glucopyranosyl ester; 3-O-[beta-D-glucopyranosyl-(1-->2)-beta-D-glucopyranosyl] oleanolic acid 28-O-beta-D-glucopyranosyl ester; 3-O-[a-L-rhamnopyranosyl-(1-->3)-beta-D-glucuronopyranosyl] oleanolic acid; 3-O-[alpha-L-rhamnopyranosyl-(1-->3)-beta-D-glucuronopyranosyl] oleanolic acid 28-O-beta-D-glucopyranosyl ester; 28-O-β-D-glucopyranosyl-oleanolic acid; 3-O-β-D-glucopyranosyl (1→3)-β-D-glucopyranosiduronic acid (CS1); oleanolic acid 3-O-β-D-glucopyranosyl (1→3)-β-D-glucopyranosiduronic acid (CS2); methyl 3,11-dioxoolean-12-en-28-olate (DIOXOL); ZCVI₄-2; Benzyl 3-dehydr-oxy-1,2,5-oxadiazolo[3′,4′:2,3]oleanolate) lung and/or heart transplantation.

In some embodiments, the present disclosure relates to methods of treating an interstitial lung disease (e.g., idiopathic pulmonary fibrosis) comprising administering to a subject in need thereof an effective amount of any of a single-arm ActRIIA heteromultimer or single-arm ActRIIB heteromultimer as disclosed herein (e.g., an antagonist of one or more of activin A, activin B, GDF11, GDF8, GDF3, BMP5, BMP6, and BMP10). In some embodiments, the interstitial lung disease is pulmonary fibrosis. In some embodiments, the interstitial lung disease is caused by any one of the following: silicosis, asbestosis, berylliosis, hypersensitivity pneumonitis, drug use (e.g., antibiotics, chemotherapeutic drugs, antiarrhythmic agents, statins), systemic sclerosis, polymyositis, dermatomyositis, systemic lupus erythematosus, rheumatoid arthritis, an infection (e.g., atypical pneumonia, pneumocystis pneumonia, tuberculosis, chlamydia trachomatis, and/or respiratory syncytial virus), lymphangitic carcinomatosis, cigarette smoking, or developmental disorders. In some embodiments, the interstitial lung disease is idiopathic (e.g., sarcoidosis, idiopathic pulmonary fibrosis, Hamman-Rich syndrome, and/or antisynthetase syndrome). In particular embodiments, the interstitial lung disease is idiopathic pulmonary fibrosis. In some embodiments, the treatment for idiopathic pulmonary fibrosis is administered in combination with an additional therapeutic agent. In some embodiments, the additional therapeutic agent is selected from the group consisting of: pirfenidone, N-acetylcysteine, prednisone, azathioprine, nintedanib, derivatives thereof and combinations thereof.

Pulmonary hypertension (PH) has been previously classified as primary (idiopathic) or secondary. Recently, the World Health Organization (WHO) has classified pulmonary hypertension into five groups: Group 1: pulmonary arterial hypertension (PAH); Group 2: pulmonary hypertension with left heart disease; Group 3: pulmonary hypertension with lung disease and/or hypoxemia; Group 4: pulmonary hypertension due to chronic thrombotic and/or embolic disease; and Group 5: miscellaneous conditions (e.g., sarcoidosis, histiocytosis X, lymphangiomatosis and compression of pulmonary vessels). See, for example, Rubin (2004) Chest 126:7-10.

In certain aspects, the disclosure relates to methods of treating, preventing, or reducing the progression rate and/or severity of pulmonary hypertension (e.g., treating, preventing, or reducing the progression rate and/or severity of one or more complications of pulmonary hypertension) comprising administering to a subject in need thereof an effective amount of a single-arm ActRIIA heteromultimer or single-arm ActRIIB heteromultimer (e.g., an antagonist of one or more of activin A, activin B, GDF11, GDF8, GDF3, BMP5, BMP6, and BMP10). In some embodiments, the method relates to pulmonary hypertension subjects that have pulmonary arterial hypertension. In some embodiments, the method relates pulmonary hypertension subjects that have pulmonary hypertension with left heart disease. In some embodiments, the method relates to pulmonary hypertension subjects that have lung disease and/or hypoxemia. In some embodiments, the method relates to pulmonary hypertension subjects that have chronic thrombotic and/or embolic disease. In some embodiments, the method relates to pulmonary hypertension subjects that have sarcoidosis, histiocytosis X, or lymphangiomatosis and compression of pulmonary vessels.

Pulmonary arterial hypertension is a serious, progressive and life-threatening disease of the pulmonary vasculature, characterized by profound vasoconstriction and an abnormal proliferation of smooth muscle cells in the walls of the pulmonary arteries. Severe constriction of the blood vessels in the lungs leads to very high pulmonary arterial pressures. These high pressures make it difficult for the heart to pump blood through the lungs to be oxygenated. Subjects with PAH suffer from extreme shortness of breath as the heart struggles to pump against these high pressures. Subjects with PAH typically develop significant increases in pulmonary vascular resistance (PVR) and sustained elevations in pulmonary artery pressure (PAP), which ultimately lead to right ventricular failure and death. Subjects diagnosed with PAH have a poor prognosis and equally compromised quality of life, with a mean life expectancy of 2 to 5 years from the time of diagnosis if untreated.

A variety of factors contribute to the pathogenesis of pulmonary hypertension including proliferation of pulmonary cells which can contribute to vascular remodeling (i.e., hyperplasia). For example, pulmonary vascular remodeling occurs primarily by proliferation of arterial endothelial cells and smooth muscle cells of subjects with pulmonary hypertension. Overexpression of various cytokines is believed to promote pulmonary hypertension. Further, it has been found that pulmonary hypertension may rise from the hyperproliferation of pulmonary arterial smooth cells and pulmonary endothelial cells. Still further, advanced PAH may be characterized by muscularization of distal pulmonary arterioles, concentric intimal thickening, and obstruction of the vascular lumen by proliferating endothelial cells. Pietra et al., J. Am. Coll. Cardiol., 43:255-325 (2004).

In certain aspects, the disclosure relates to methods of treating, preventing, or reducing the progression rate and/or severity of pulmonary hypertension (e.g., treating, preventing, or reducing the progression rate and/or severity of one or more complications of pulmonary hypertension) comprising administering to a subject in need thereof an effective amount of a single-arm ActRIIA heteromultimer or single-arm ActRIIB heteromultimer (e.g., an antagonist of one or more of activin A. activin B, GDF11, GDF8, GDF3, BMP5, BMP6, BMP10), wherein the subject has resting pulmonary arterial pressure (PAP) of at least 25 mm Hg (e.g., 25, 30, 35, 40, 45, or 50 mm Hg). In some embodiments, the method relates to subjects having a resting PAP of at least 25 mm Hg. In some embodiments, the method relates to subjects having a resting PAP of at least 30 mm Hg. In some embodiments, the method relates to subjects having a resting PAP of at least 35 mm Hg. In some embodiments, the method relates to subjects having a resting PAP of at least 40 mm Hg. In some embodiments, the method relates to subjects having a resting PAP of at least 45 mm Hg. In some embodiments, the method relates to subjects having a resting PAP of at least 50 mm Hg.

In some embodiments, the disclosure relates to methods of adjusting one or more hemodynamic parameters in the PH subject toward a more normal level (e.g., normal as compared to healthy people of similar age and sex), comprising administering to a subject in need thereof an effective amount of a single-arm ActRIIA heteromultimer or single-arm ActRIIB heteromultimer (e.g., an antagonist of one or more of activin A, activin B, GDF11, GDF8, GDF3, BMP5, BMP6, and BMP10). In some embodiments, the method relates to reducing PAP. In some embodiments, the method relates to reducing the subject’s PAP by at least 3 mmHg. In certain embodiments, the method relates to reducing the subject’s PAP by at least 5 mmHg. In certain embodiments, the method relates to reducing the subject’s PAP by at least 7 mmHg. In certain embodiments, the method relates to reducing the subject’s PAP by at least 10 mmHg. In certain embodiments, the method relates to reducing the subject’s PAP by at least 12 mmHg. In certain embodiments, the method relates to reducing the subject’s PAP by at least 15 mmHg. In certain embodiments, the method relates to reducing the subject’s PAP by at least 20 mmHg. In certain embodiments, the method relates to reducing the subject’s PAP by at least 25 mmHg. In some embodiments, the method relates to decreasing pulmonary arterial pressure in a subject. In some embodiments, the method relates to decreasing pulmonary arterial pressure in a subject by at least 10% (e.g., 15%, 20%, 25%, 30%, 35%, 40%, 45%, 50%, 55%, 60%, 65%, 70%, 75%, or 80%). In some embodiments, the method relates to decreasing pulmonary arterial pressure in a subject by at least 15%. In some embodiments, the method relates to decreasing pulmonary arterial pressure in a subject by at least 15%. In some embodiments, the method relates to decreasing pulmonary arterial pressure in a subject by at least 20%. In some embodiments, the method relates to decreasing pulmonary arterial pressure in a subject by at least 25%. In some embodiments, the method relates to decreasing pulmonary arterial pressure in a subject by at least 30%. In some embodiments, the method relates to decreasing pulmonary arterial pressure in a subject by at least 40%. In some embodiments, the method relates to decreasing pulmonary arterial pressure in a subject by at least 45%. In some embodiments, the method relates to decreasing pulmonary arterial pressure in a subject by at least 50%. In some embodiments, the method relates to decreasing pulmonary arterial pressure in a subject by at least 55%. In some embodiments, the method relates to decreasing pulmonary arterial pressure in a subject by at least 60%. In some embodiments, the method relates to decreasing pulmonary arterial pressure in a subject by at least 70%. In some embodiments, the method relates to decreasing pulmonary arterial pressure in a subject by at least 75%. In some embodiments, the method relates to decreasing pulmonary arterial pressure in a subject by at least 80%. In some embodiments, the method relates to reducing pulmonary vascular resistance (PVR). In some embodiments, the method relate to increasing pulmonary capillary wedge pressure (PCWP). In some embodiments, the method relate to increasing left ventricular end-diastolic pressure (LVEDP).

In some embodiments, the method relates to decreasing ventricle hypertrophy in a subject. In some embodiments, the method relates to decreasing ventricle hypertrophy in a subject by at least 10% (e.g., 15%, 20%, 25%, 30%, 35%, 40%, 45%, 50%, 55%, 60%, 65%, 70%, 75%, or 80%). In some embodiments, the method relates to decreasing ventricle hypertrophy in a subject by at least 15%. In some embodiments, the method relates to decreasing ventricle hypertrophy in a subject by at least 15%. In some embodiments, the method relates to decreasing ventricle hypertrophy in a subject by at least 20%. In some embodiments, the method relates to decreasing ventricle hypertrophy in a subject by at least 25%. In some embodiments, the method relates to decreasing ventricle hypertrophy in a subject by at least 30%. In some embodiments, the method relates to decreasing ventricle hypertrophy in a subject by at least 40%. In some embodiments, the method relates to decreasing ventricle hypertrophy in a subject by at least 45%. In some embodiments, the method relates to decreasing ventricle hypertrophy in a subject by at least 50%. In some embodiments, the method relates to decreasing ventricle hypertrophy in a subject by at least 55%. In some embodiments, the method relates to decreasing ventricle hypertrophy in a subject by at least 60%. In some embodiments, the method relates to decreasing ventricle hypertrophy in a subject by at least 70%. In some embodiments, the method relates to decreasing ventricle hypertrophy in a subject by at least 75%. In some embodiments, the method relates to decreasing ventricle hypertrophy in a subject by at least 80%.

In some embodiments, the method relates to decreasing smooth muscle hypertrophy in a subject. In some embodiments, the method relates to decreasing smooth muscle hypertrophy in a subject by at least 10% (e.g., 15%, 20%, 25%, 30%, 35%, 40%, 45%, 50%, 55%, 60%, 65%, 70%, 75%, or 80%). In some embodiments, the method relates to decreasing smooth muscle hypertrophy in a subject by at least 15%. In some embodiments, the method relates to decreasing smooth muscle hypertrophy in a subject by at least 15%. In some embodiments, the method relates to decreasing smooth muscle hypertrophy in a subject by at least 20%. In some embodiments, the method relates to decreasing smooth muscle hypertrophy in a subject by at least 25%. In some embodiments, the method relates to decreasing smooth muscle hypertrophy in a subject by at least 30%. In some embodiments, the method relates to decreasing smooth muscle hypertrophy in a subject by at least 40%. In some embodiments, the method relates to decreasing smooth muscle hypertrophy in a subject by at least 45%. In some embodiments, the method relates to decreasing smooth muscle hypertrophy in a subject by at least 50%. In some embodiments, the method relates to decreasing smooth muscle hypertrophy in a subject by at least 55%. In some embodiments, the method relates to decreasing smooth muscle hypertrophy in a subject by at least 60%. In some embodiments, the method relates to decreasing smooth muscle hypertrophy in a subject by at least 70%. In some embodiments, the method relates to decreasing smooth muscle hypertrophy in a subject by at least 75%. In some embodiments, the method relates to decreasing smooth muscle hypertrophy in a subject by at least 80%.

In some embodiments, the method relates to decreasing pulmonary arteriole muscularity in a subject. In some embodiments, the method relates to decreasing pulmonary arteriole muscularity in a subject by at least 10% (e.g., 15%, 20%, 25%, 30%, 35%, 40%, 45%, 50%, 55%, 60%, 65%, 70%, 75%, or 80%). In some embodiments, the method relates to decreasing pulmonary arteriole muscularity in a subject by at least 15%. In some embodiments, the method relates to decreasing pulmonary arteriole muscularity in a subject by at least 15%. In some embodiments, the method relates to decreasing pulmonary arteriole muscularity in a subject by at least 20%. In some embodiments, the method relates to decreasing pulmonary arteriole muscularity in a subject by at least 25%. In some embodiments, the method relates to decreasing pulmonary arteriole muscularity in a subject by at least 30%. In some embodiments, the method relates to decreasing pulmonary arteriole muscularity in a subject by at least 40%. In some embodiments, the method relates to decreasing pulmonary arteriole muscularity in a subject by at least 45%. In some embodiments, the method relates to decreasing pulmonary arteriole muscularity in a subject by at least 50%. In some embodiments, the method relates to decreasing pulmonary arteriole muscularity in a subject by at least 55%. In some embodiments, the method relates to decreasing pulmonary arteriole muscularity in a subject by at least 60%. In some embodiments, the method relates to decreasing pulmonary arteriole muscularity in a subject by at least 70%. In some embodiments, the method relates to decreasing pulmonary arteriole muscularity in a subject by at least 75%. In some embodiments, the method relates to decreasing pulmonary arteriole muscularity in a subject by at least 80%.

In some embodiments, the method relates to decreasing pulmonary vascular resistance in a subject. In some embodiments, the method relates to decreasing pulmonary vascular resistance in a subject by at least 10% (e.g., 15%, 20%, 25%, 30%, 35%, 40%, 45%, 50%, 55%, 60%, 65%, 70%, 75%, or 80%). In some embodiments, the method relates to decreasing pulmonary vascular resistance in a subject by at least 15%. In some embodiments, the method relates to decreasing pulmonary vascular resistance in a subject by at least 15%. In some embodiments, the method relates to decreasing pulmonary vascular resistance in a subject by at least 20%. In some embodiments, the method relates to decreasing pulmonary vascular resistance in a subject by at least 25%. In some embodiments, the method relates to decreasing pulmonary vascular resistance in a subject by at least 30%. In some embodiments, the method relates to decreasing pulmonary vascular resistance in a subject by at least 40%. In some embodiments, the method relates to decreasing pulmonary vascular resistance in a subject by at least 45%. In some embodiments, the method relates to decreasing pulmonary vascular resistance in a subject by at least 50%. In some embodiments, the method relates to decreasing pulmonary vascular resistance in a subject by at least 55%. In some embodiments, the method relates to decreasing pulmonary vascular resistance in a subject by at least 60%. In some embodiments, the method relates to decreasing pulmonary vascular resistance in a subject by at least 70%. In some embodiments, the method relates to decreasing pulmonary vascular resistance in a subject by at least 75%. In some embodiments, the method relates to decreasing pulmonary vascular resistance in a subject by at least 80%.

In certain aspects, the disclosure relates to methods of treating, preventing, or reducing the progression rate and/or severity of one or more complications of pulmonary hypertension comprising administering to a subject in need thereof an effective amount of a single-arm ActRIIA heteromultimer or single-arm ActRIIB heteromultimer (e.g., an antagonist of one or more of activin A, activin B, GDF11, GDF11, GDF3, BMP5, BMP6, BMP10,). In some embodiments, the method relates to treating, preventing, or reducing the progression rate and/or severity of cell proliferation in the pulmonary artery of a pulmonary hypertension subject. In some embodiments, the method relates to treating, preventing, or reducing the progression rate and/or severity of smooth muscle and/or endothelial cells proliferation in the pulmonary artery of a pulmonary hypertension subject. In some embodiments, the method relates to treating, preventing, or reducing the progression rate and/or severity of angiogenesis in the pulmonary artery of a pulmonary hypertension subject. In some embodiments, the method relates to increasing physical activity of a subject having pulmonary hypertension. In some embodiments, the method relates to treating, preventing, or reducing the progression rate and/or severity of dyspnea in a pulmonary hypertension subject. In some embodiments, the method relates to treating, preventing, or reducing the progression rate and/or severity of chest pain in a pulmonary hypertension subject. In some embodiments, the method relates to treating, preventing, or reducing the progression rate and/or severity of fatigue in a pulmonary hypertension subject. In some embodiments, the method relates to treating, preventing, or reducing the progression rate and/or severity of pulmonary fibrosis in a pulmonary hypertension subject. In some embodiments, the method relates to treating, preventing, or reducing the progression rate and/or severity of fibrosis in a pulmonary hypertension subject. In some embodiments, the method relates to treating, preventing, or reducing the progression rate and/or severity of pulmonary vascular remodeling in a pulmonary hypertension subject. In some embodiments, the method relates to treating, preventing, or reducing the progression rate and/or severity of right ventricular hypertrophy in a pulmonary hypertension subject.

In certain aspects, the disclosure relates to methods of increasing exercise capacity in a subject having pulmonary hypertension comprising administering to a subject in need thereof an effective amount of a single-arm ActRIIA heteromultimer or single-arm ActRIIB heteromultimer (e.g., an antagonist of one or more of activin A, activin B, GDF11, GDF8, GDF3, BMP5, BMP6, BMP10). Any suitable measure of exercise capacity can be used. For example, exercise capacity in a 6-minute walk test (6MWT), which measures how far the subject can walk in 6 minutes, i.e., the 6-minute walk distance (6MWD), is frequently used to assess pulmonary hypertension severity and disease progression. In some embodiments, a subject has a 6MWD from 150 to 400 meters. In some embodiments, the method relates to increasing a subject’s 6MWD. In some embodiments, the method relates to increasing 6MWD by at least 10 meters in the subject having pulmonary hypertension. In some embodiments, the method relates to increasing 6MWD by at least 20 meters in the subject having pulmonary hypertension. In some embodiments, the method relates to increasing 6MWD by at least 30 meters in the subject having pulmonary hypertension. In some embodiments, the method relates to increasing 6MWD by at least 40 meters in the subject having pulmonary hypertension. In some embodiments, the method relates to increasing 6MWD by at least 50 meters in the subject having pulmonary hypertension. In some embodiments, the method relates to increasing 6MWD by at least 60 meters in the subject having pulmonary hypertension. In some embodiments, the method relates to increasing 6MWD by at least 70 meters in the subject having pulmonary hypertension. In some embodiments, the method relates to increasing 6MWD by at least 80 meters in the subject having pulmonary hypertension. In some embodiments, the method relates to increasing 6MWD by at least 90 meters in the subject having pulmonary hypertension. In some embodiments, the method relates to increasing 6MWD by at least 100 meters in the subject having pulmonary hypertension. The Borg dyspnea index (BDI) is a numerical scale for assessing perceived dyspnea (breathing discomfort). It measures the degree of breathlessness, for example, after completion of the 6MWT, where a BDI of 0 indicates no breathlessness and 10 indicates maximum breathlessness. In some embodiments, the method relates to reducing the subject’s Borg dyspnea index (BDI). In some embodiments, the method relates to lowering BDI by at least 0.5 index points in the subject having pulmonary hypertension. In some embodiments, the method relates to lowering BDI by at least 1 index points in the subject having pulmonary hypertension. In some embodiments, the method relates to lowering BDI by at least 1.5 index points in the subject having pulmonary hypertension. In some embodiments, the method relates to lowering BDI by at least 2 index points in the subject having pulmonary hypertension. In some embodiments, the method relates to lowering BDI by at least 2.5 index points in the subject having pulmonary hypertension. In some embodiments, the method relates to lowering BDI by at least 3 index points in the subject having pulmonary hypertension. In some embodiments, the method relates to lowering BDI by at least 3.5 index points in the subject having pulmonary hypertension. In some embodiments, the method relates to lowering BDI by at least 4 index points in the subject having pulmonary hypertension. In some embodiments, the method relates to lowering BDI by at least 4.5 index points in the subject having pulmonary hypertension. In some embodiments, the method relates to lowering BDI by at least 5 index points in the subject having pulmonary hypertension. In some embodiments, the method relates to lowering BDI by at least 5.5 index points in the subject having pulmonary hypertension. In some embodiments, the method relates to lowering BDI by at least 6 index points in the subject having pulmonary hypertension. In some embodiments, the method relates to lowering BDI by at least 6.5 index points in the subject having pulmonary hypertension. In some embodiments, the method relates to lowering BDI by at least 7 index points in the subject having pulmonary hypertension. In some embodiments, the method relates to lowering BDI by at least 7.5 index points in the subject having pulmonary hypertension. In some embodiments, the method relates to lowering BDI by at least 8 index points in the subject having pulmonary hypertension. In some embodiments, the method relates to lowering BDI by at least 8.5 index points in the subject having pulmonary hypertension. In some embodiments, the method relates to lowering BDI by at least 9 index points in the subject having pulmonary hypertension. In some embodiments, the method relates to lowering BDI by at least 9.5 index points in the subject having pulmonary hypertension. In some embodiments, the method relates to lowering BDI by at least 3 index points in the subject having pulmonary hypertension. In some embodiments, the method relates to lowering BDI by 10 index points in the subject having pulmonary hypertension.

Pulmonary hypertension at baseline can be mild, moderate or severe, as measured for example by World Health Organization (WHO) functional class, which is a measure of disease severity in subjects with pulmonary hypertension. The WHO functional classification is an adaptation of the New York Heart Association (NYHA) system and is routinely used to qualitatively assess activity tolerance, for example in monitoring disease progression and response to treatment (Rubin (2004) Chest 126:7-10). Four functional classes are recognized in the WHO system: Class I: pulmonary hypertension without resulting limitation of physical activity; ordinary physical activity does not cause undue dyspnea or fatigue, chest pain or near syncope; Class II: pulmonary hypertension resulting in slight limitation of physical activity; subject comfortable at rest; ordinary physical activity causes undue dyspnea or fatigue, chest pain or near syncope; Class III: pulmonary hypertension resulting in marked limitation of physical activity; subject comfortable at rest; less than ordinary activity causes undue dyspnea or fatigue, chest pain or near syncope; Class IV: pulmonary hypertension resulting in inability to carry out any physical activity without symptoms; subject manifests signs of right-heart failure; dyspnea and/or fatigue may be present even at rest; discomfort is increased by any physical activity.

In certain aspects, the disclosure relates to methods of treating, preventing, or reducing the progression rate and/or severity of pulmonary hypertension (e.g., treating, preventing, or reducing the progression rate and/or severity of one or more complications of pulmonary hypertension) comprising administering to a subject in need thereof an effective amount of a single-arm ActRIIA heteromultimer or single-arm ActRIIB heteromultimer (e.g., an antagonist of one or more of activin A, activin B, GDF11, GDF8, GDF3, BMP5, BMP6, and BMP10), wherein the subject has Class I, Class II, Class III, or Class IV pulmonary hypertension as recognized by the WHO. In some embodiments, the method relates to a subject that has Class I pulmonary hypertension as recognized by the WHO. In some embodiments, the method relates to a subject that has Class II pulmonary hypertension as recognized by the WHO. In some embodiments, the method relates to preventing or delaying subject progression from Class I pulmonary hypertension to Class II pulmonary hypertension as recognized by the WHO. In some embodiments, the method relates to promoting or increasing subject regression from Class II pulmonary hypertension to Class I pulmonary hypertension as recognized by the WHO. In some embodiments, the method relates to a subject that has Class III pulmonary hypertension as recognized by the WHO. In some embodiments, the method relates to preventing or delaying subject progression from Class II pulmonary hypertension to Class III pulmonary hypertension as recognized by the WHO. In some embodiments, the method relates to promoting or increasing subject regression from Class III pulmonary hypertension to Class II pulmonary hypertension as recognized by the WHO. In some embodiments, the method relates to promoting or increasing subject regression from Class III pulmonary hypertension to Class I pulmonary hypertension as recognized by the WHO. In some embodiments, the method relates to a subject that has Class IV pulmonary hypertension as recognized by the WHO. In some embodiments, the method relates to preventing or delaying subject progression from Class III pulmonary hypertension to Class IV pulmonary hypertension as recognized by the WHO. In some embodiments, the method relates to promoting or increasing subject regression from Class IV pulmonary hypertension to Class III pulmonary hypertension as recognized by the WHO. In some embodiments, the method relates to promoting or increasing subject regression from Class IV pulmonary hypertension to Class II pulmonary hypertension as recognized by the WHO. In some embodiments, the method relates to promoting or increasing subject regression from Class IV pulmonary hypertension to Class I pulmonary hypertension as recognized by the WHO.

There is no known cure for pulmonary hypertension; current methods of treatment focus on prolonging subject lifespan and enhancing subject quality of life. Current methods of treatment of pulmonary hypertension include administration of: vasodilators such as prostacyclin, epoprostenol, and sildenafil; endothelin receptor antagonists such as bosentan; calcium channel blockers such as amlodipine, diltiazem, and nifedipine; anticoagulants such as warfarin; and diuretics. Treatment of pulmonary hypertension has also been carried out using oxygen therapy, atrial septostomy, pulmonary thromboendarterectomy, and lung and/or heart transplantation. Each of these methods, however, suffers from one or multiple drawbacks which may include lack of effectiveness, serious side effects, low subject compliance, and high cost. In certain aspects, the method relate to treating, preventing, or reducing the progression rate and/or severity of pulmonary hypertension (e.g., treating, preventing, or reducing the progression rate and/or severity of one or more complications of pulmonary hypertension) comprising administering to a subject in need thereof an effective amount of a single-arm ActRIIA heteromultimer or single-arm ActRIIB heteromultimer (e.g., an antagonist of one or more of activin A, activin B, GDF11, GDF8, GDF3, BMP5, BMP6, and BMP10) in combination (e.g., administered at the same time or different times, but generally in such a manner as to achieve overlapping pharmacological/physiological effects) with one or more additional active agents and/or supportive therapies for treating pulmonary hypertension (e.g., vasodilators such as prostacyclin, epoprostenol, and sildenafil; endothelin receptor antagonists such as bosentan; calcium channel blockers such as amlodipine, diltiazem, and nifedipine; anticoagulants such as warfarin; diuretics; oxygen therapy; atrial septostomy; pulmonary thromboendarterectomy: and lung and/or heart transplantation); bardoxolone methyl or a derivative thereof; oleanolic acid or derivative thereof.

In certain embodiments, the present disclosure provides methods for managing a subject that has been treated with, or is a candidate to be treated with, one or more one or more a single-arm ActRIIA heteromultimers or single-arm ActRIIB heteromultimers of the disclosure by measuring one or more hematologic parameters in the subject. The hematologic parameters may be used to evaluate appropriate dosing for a subject who is a candidate to be treated with the antagonist of the present disclosure, to monitor the hematologic parameters during treatment, to evaluate whether to adjust the dosage during treatment with one or more antagonist of the disclosure, and/or to evaluate an appropriate maintenance dose of one or more antagonists of the disclosure. If one or more of the hematologic parameters are outside the normal level, dosing with one or more a single-arm ActRIIA heteromultimers or single-arm ActRIIB heteromultimers of the disclosure may be reduced, delayed or terminated.

Hematologic parameters that may be measured in accordance with the methods provided herein include, for example, red blood cell levels, blood pressure, iron stores, and other agents found in bodily fluids that correlate with increased red blood cell levels, using art recognized methods. Such parameters may be determined using a blood sample from a subject. Increases in red blood cell levels, hemoglobin levels, and/or hematocrit levels may cause increases in blood pressure.

In one embodiment, if one or more hematologic parameters are outside the normal range or on the high side of normal in a subject who is a candidate to be treated with one or more single-arm ActRIIA heteromultimers or single-arm ActRIIB heteromultimers, then onset of administration of the one or more antagonists of the disclosure may be delayed until the hematologic parameters have returned to a normal or acceptable level either naturally or via therapeutic intervention. For example, if a candidate subject is hypertensive or pre-hypertensive, then the subject may be treated with a blood pressure lowering agent in order to reduce the subject’s blood pressure. Any blood pressure lowering agent appropriate for the individual subject’s condition may be used including, for example, diuretics, adrenergic inhibitors (including alpha blockers and beta blockers), vasodilators, calcium channel blockers, angiotensin-converting enzyme (ACE) inhibitors, or angiotensin II receptor blockers. Blood pressure may alternatively be treated using a diet and exercise regimen. Similarly, if a candidate subject has iron stores that are lower than normal, or on the low side of normal, then the subject may be treated with an appropriate regimen of diet and/or iron supplements until the subject’s iron stores have returned to a normal or acceptable level. For subjects having higher than normal red blood cell levels and/or hemoglobin levels, then administration of the one or more antagonists of the disclosure may be delayed until the levels have returned to a normal or acceptable level.

In certain embodiments, if one or more hematologic parameters are outside the normal range or on the high side of normal in a subject who is a candidate to be treated with one or more a single-arm ActRIIA heteromultimers or single-arm ActRIIB heteromultimers, then the onset of administration may not be delayed. However, the dosage amount or frequency of dosing of the one or more antagonists of the disclosure may be set at an amount that would reduce the risk of an unacceptable increase in the hematologic parameters arising upon administration of the one or more antagonists of the disclosure. Alternatively, a therapeutic regimen may be developed for the subject that combines one or more a single-arm ActRIIA heteromultimers or single-arm ActRIIB heteromultimers with a therapeutic agent that addresses the undesirable level of the hematologic parameter. For example, if the subject has elevated blood pressure, then a therapeutic regimen may be designed involving administration of one or more a single-arm ActRIIA heteromultimers or single-arm ActRIIB heteromultimers and a blood pressure lowering agent. For a subject having lower than desired iron stores, a therapeutic regimen may be developed involving one or more a single-arm ActRIIA heteromultimers or single-arm ActRIIB heteromultimers of the disclosure and iron supplementation.

In one embodiment, baseline parameter(s) for one or more hematologic parameters may be established for a subject who is a candidate to be treated with one or more a single-arm ActRIIA heteromultimers or single-arm ActRIIB heteromultimers of the disclosure and an appropriate dosing regimen established for that subject based on the baseline value(s). Alternatively, established baseline parameters based on a subject’s medical history could be used to inform an appropriate antagonist dosing regimen for a subject. For example, if a healthy subject has an established baseline blood pressure reading that is above the defined normal range it may not be necessary to bring the subject’s blood pressure into the range that is considered normal for the general population prior to treatment with the one or more antagonist of the disclosure. A subject’s baseline values for one or more hematologic parameters prior to treatment with one or more a single-arm ActRIIA heteromultimers or single-arm ActRIIB heteromultimers of the disclosure may also be used as the relevant comparative values for monitoring any changes to the hematologic parameters during treatment with the one or more antagonists of the disclosure.

In certain embodiments, one or more hematologic parameters are measured in subjects who are being treated with one or more a single-arm ActRIIA heteromultimers or single-arm ActRIIB heteromultimers. The hematologic parameters may be used to monitor the subject during treatment and permit adjustment or termination of the dosing with the one or more a single-arm ActRIIA heteromultimers or single-arm ActRIIB heteromultimers of the disclosure or additional dosing with another therapeutic agent. For example, if administration of one or more a single-arm ActRIIA heteromultimers or single-arm ActRIIB heteromultimers results in an increase in blood pressure, red blood cell level, or hemoglobin level, or a reduction in iron stores, then the dose of the one or more antagonists of the disclosure may be reduced in amount or frequency in order to decrease the effects of the one or more antagonists of the disclosure on the one or more hematologic parameters. In some embodiments, a subject has a hemoglobin level from >8 and <15 d/dl. If administration of one or more a single-arm ActRIIA heteromultimers or single-arm ActRIIB heteromultimers results in a change in one or more hematologic parameters that is adverse to the subject, then the dosing of the one or more antagonists of the disclosure may be terminated either temporarily, until the hematologic parameter(s) return to an acceptable level, or permanently. Similarly, if one or more hematologic parameters are not brought within an acceptable range after reducing the dose or frequency of administration of the one or more antagonists of the disclosure, then the dosing may be terminated. As an alternative, or in addition to, reducing or terminating the dosing with the one or more antagonists of the disclosure, the subject may be dosed with an additional therapeutic agent that addresses the undesirable level in the hematologic parameter(s), such as, for example, a blood pressure lowering agent or an iron supplement. For example, if a subject being treated with one or more a single-arm ActRIIA heteromultimers or single-arm ActRIIB heteromultimers has elevated blood pressure, then dosing with the one or more antagonists of the disclosure may continue at the same level and a blood-pressure-lowering agent is added to the treatment regimen, dosing with the one or more antagonist of the disclosure may be reduced (e.g., in amount and/or frequency) and a blood-pressure-lowering agent is added to the treatment regimen, or dosing with the one or more antagonist of the disclosure may be terminated and the subject may be treated with a blood-pressure-lowering agent.

In some embodiments, a subject has pulmonary arterial hypertension and has Functional Class II or Class III pulmonary hypertension in accordance with the World Health Organization’s functional classification system for pulmonary hypertension. In some embodiments, a subject has pulmonary arterial hypertension that is classified as one or more subtypes selected from the group consisting of: idiopathic or heritable pulmonary arterial hypertension, drug- and/or toxin-induced pulmonary hypertension, pulmonary hypertension associated with connective tissue disease, and pulmonary hypertension associated with congenital systemic-to-pulmonary shunts at least 1 year following shunt repair. In some embodiments, a subject has pulmonary arterial hypertension that is classified as idiopathic or heritable pulmonary arterial hypertension. In some embodiments, a subject has pulmonary arterial hypertension that is classified as drug- and/or toxin-induced pulmonary hypertension. In some embodiments, a subject has pulmonary arterial hypertension that is classified as pulmonary hypertension associated with connective tissue disease. In some embodiments, a subject has pulmonary arterial hypertension that is classified as pulmonary hypertension associated with congenital systemic-to-pulmonary shunts at least 1 year following shunt repair. In some embodiments, the subject has been treated with one or more vasodilators. In some embodiments, a subject has been treated with one or more agents selected from the group consisting of: phosphodiesterase type 5 inhibitors, soluble guanylate cyclase stimulators, prostacyclin receptor agonist, and endothelin receptor antagonists. In some embodiments, one or more agents is selected from the group consisting of: bosentan, sildenafil, beraprost, macitentan, selexipag, epoprostenol, treprostinil, iloprost, ambrisentan, and tadalafil. In some embodiments, method of the present disclosure further comprise administration of one or more vasodilators. In some embodiments, methods of the disclosure further comprise administration of one or more agents selected from the group consisting of: phosphodiesterase type 5 inhibitors, soluble guanylate cyclase stimulators, prostacyclin receptor agonist, and endothelin receptor antagonists. In some embodiments, one or more agents is selected from the group consisting of: bosentan, sildenafil, beraprost, macitentan, selexipag, epoprostenol, treprostinil, iloprost, ambrisentan, and tadalafil.

In some embodiments, methods of the present disclosure delay clinical worsening of pulmonary arterial hypertension in a subject. In some embodiments, methods of the present disclosure delay clinical worsening of pulmonary hypertension in accordance with the World Health Organization’s functional classification system for pulmonary hypertension. In some embodiments, methods of the present disclosure reduce the risk of hospitalization for one or more complications associated with pulmonary arterial hypertension.

### 7. Pharmaceutical Compositions

In certain aspects, a single-arm ActRIIA heteromultimers or single-arm ActRIIB heteromultimers of the present disclosure can be administered alone or as a component of a pharmaceutical formulation (also referred to as a therapeutic composition or pharmaceutical composition). A pharmaceutical formation refers to a preparation which is in such form as to permit the biological activity of an active ingredient (e.g., an agent of the present disclosure) contained therein to be effective and which contains no additional components which are unacceptably toxic to a subject to which the formulation would be administered. The subject compounds may be formulated for administration in any convenient way for use in human or veterinary medicine. For example, one or more agents of the present disclosure may be formulated with a pharmaceutically acceptable carrier. A pharmaceutically acceptable carrier refers to an ingredient in a pharmaceutical formulation, other than an active ingredient, which is generally nontoxic to a subject. A pharmaceutically acceptable carrier includes, but is not limited to, a buffer, excipient, stabilizer, and/or preservative. In general, pharmaceutical formulations for use in the present disclosure are in a pyrogen-free, physiologically-acceptable form when administered to a subject. Therapeutically useful agents other than those described herein, which may optionally be included in the formulation as described above, may be administered in combination with the subject agents in the methods of the present disclosure.

In certain embodiments, the therapeutic methods of the disclosure include administering the composition systemically, or locally as an implant or device. When administered, the therapeutic composition for use in this disclosure is in a substantially pyrogen-free, or pyrogen-free, physiologically acceptable form. Therapeutically useful agents other than the a single-arm ActRIIA heteromultimers or single-arm ActRIIB heteromultimers which may also optionally be included in the composition as described above, may be administered simultaneously or sequentially with the subject compounds in the methods disclosed herein.

Typically, protein therapeutic agents disclosed herein will be administered parentally, and particularly intravenously or subcutaneously. Pharmaceutical compositions suitable for parenteral administration may comprise one or more a single-arm ActRIIA heteromultimers or single-arm ActRIIB heteromultimers in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The compositions and formulations may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration

Further, the composition may be encapsulated or injected in a form for delivery to a target tissue site. In certain embodiments, compositions of the present invention may include a matrix capable of delivering one or more therapeutic compounds (e.g., a single-arm ActRIIA heteromultimers or single-arm ActRIIB heteromultimers) to a target tissue site, providing a structure for the developing tissue and optimally capable of being resorbed into the body. For example, the matrix may provide slow release of the a single-arm ActRIIA heteromultimer or single-arm ActRIIB heteromultimer. Such matrices may be formed of materials presently in use for other implanted medical applications.

The choice of matrix material is based on biocompatibility, biodegradability, mechanical properties, cosmetic appearance and interface properties. The particular application of the subject compositions will define the appropriate formulation. Potential matrices for the compositions may be biodegradable and chemically defined calcium sulfate, tricalcium phosphate, hydroxyapatite, polylactic acid and polyanhydrides. Other potential materials are biodegradable and biologically well defined, such as bone or dermal collagen. Further matrices are comprised of pure proteins or extracellular matrix components. Other potential matrices are non-biodegradable and chemically defined, such as sintered hydroxyapatite, bioglass, aluminates, or other ceramics. Matrices may be comprised of combinations of any of the above mentioned types of material, such as polylactic acid and hydroxyapatite or collagen and tricalcium phosphate. The bioceramics may be altered in composition, such as in calcium-aluminate-phosphate and processing to alter pore size, particle size, particle shape, and biodegradability.

In solid dosage forms for oral administration (capsules, tablets, pills, dragees, powders, granules, and the like), one or more therapeutic compounds of the present invention may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

The compositions of the invention may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

It is understood that the dosage regimen will be determined by the attending physician considering various factors which modify the action of the subject compounds of the disclosure (e.g., a single-arm ActRIIA heteromultimer or single-arm ActRIIB heteromultimer). The various factors include, but are not limited to, the subject’s age, sex, and diet, the severity disease, time of administration, and other clinical factors. Optionally, the dosage may vary with the type of matrix used in the reconstitution and the types of compounds in the composition. The addition of other known growth factors to the final composition, may also affect the dosage. Progress can be monitored by periodic assessment of bone growth and/or repair, for example, X-rays (including DEXA), histomorphometric determinations, and tetracycline labeling.

In certain embodiments, the present invention also provides gene therapy for the in vivo production of a single-arm ActRIIA heteromultimers or single-arm ActRIIB heteromultimers. Such therapy would achieve its therapeutic effect by introduction of the a single-arm ActRIIA heteromultimer or single-arm ActRIIB heteromultimer polynucleotide sequences into cells or tissues having the disorders as listed above. Delivery of a single-arm ActRIIA heteromultimer or single-arm ActRIIB heteromultimer polynucleotide sequences can be achieved using a recombinant expression vector such as a chimeric virus or a colloidal dispersion system. Preferred for therapeutic delivery of a single-arm ActRIIA heteromultimer or single-arm ActRIIB heteromultimer polynucleotide sequences is the use of targeted liposomes.

In certain embodiments, the present disclosure also provides gene therapy for the in vivo production of one or more of the agents of the present disclosure. Such therapy would achieve its therapeutic effect by introduction of the agent sequences into cells or tissues having one or more of the disorders as listed above. Delivery of the agent sequences can be achieved, for example, by using a recombinant expression vector such as a chimeric virus or a colloidal dispersion system. Preferred therapeutic delivery of one or more of agent sequences of the disclosure is the use of targeted liposomes.

Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. Retroviral vectors can be made target-specific by attaching, for example, a sugar, a glycolipid, or a protein. Preferred targeting is accomplished by using an antibody. Those of skill in the art will recognize that specific polynucleotide sequences can be inserted into the retroviral genome or attached to a viral envelope to allow target specific delivery of the retroviral vector containing an a single-arm ActRIIA heteromultimer or single-arm ActRIIB heteromultimer. In a preferred embodiment, the vector is targeted to bone or cartilage.

Alternatively, tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium.

Another targeted delivery system for single-arm ActRIIA heteromultimer or single-arm ActRIIB heteromultimer polynucleotides, is a colloidal dispersion system. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. RNA, DNA and intact virions can be encapsulated within the aqueous interior and be delivered to cells in a biologically active form (see e.g., Fraley, et al., Trends Biochem. Sci., 6:77, 1981). Methods for efficient gene transfer using a liposome vehicle, are known in the art, see e.g., Mannino, et al., Biotechniques, 6:682, 1988. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Illustrative phospholipids include egg phosphatidylcholine, dipalmitoylphosphatidylcholine, and distearoylphosphatidylcholine. The targeting of liposomes is also possible based on, for example, organ-specificity, cell-specificity, and organelle-specificity and is known in the art.

The disclosure provides formulations that may be varied to include acids and bases to adjust the pH; and buffering agents to keep the pH within a narrow range.

### EXEMPLIFICATION

The invention now being generally described, it will be more readily understood by reference to the following examples, which are included merely for purposes of illustration of certain embodiments of the present invention, and are not intended to limit the invention.

### Example 1. Generation and Characterization of a Single-Arm ActRIIB Heterodimer Fc Fusion

Applicants constructed a soluble single-arm ActRIIB heterodimer Fc fusion comprising a constant region from an IgG heavy chain (e.g., Fc domain) with a short N-terminal extension and a second polypeptide in which the extracellular domain of human ActRIIB was fused to a separate constant region from an IgG heavy chain (e.g., Fc domain) with a linker positioned between the extracellular domain and this second constant region from an IgG heavy chain (e.g., Fc domain). The individual constructs are referred to as monomeric Fc polypeptide and single-arm ActRIIB Fc fusion monomer, respectively, and the sequences for each are provided below.

A methodology for promoting formation of single-arm ActRIIB heterodimer Fc fusions rather than ActRIIB homodimer Fc fusions or Fc homodimeric fusions is to introduce alterations in the amino acid sequence of the Fc domains to guide the formation of asymmetric heteromeric complexes. Many different approaches to making asymmetric interaction pairs using Fc domains are described in this disclosure.

In one approach, illustrated in the single-arm ActRIIB Fc fusion monomer and monomeric Fc polypeptide sequences of SEQ ID NOs: 46-48 and 49-51, respectively, one Fc domain is altered to introduce cationic amino acids at the interaction face, while the other Fc domain is altered to introduce anionic amino acids at the interaction face. The single-arm ActRIIB Fc fusion monomer and monomeric Fc polypeptide each employ the tissue plasminogen activator (TPA) leader: MDAMKRGLCCVLLLCGAVFVS P (SEQ ID NO: 45).

The single-arm ActRIIB Fc fusion monomer sequence (SEQ ID NO: 46) is shown below:

1 MDAMKRGLCC VLLLCGAVFV SPGASGRGEA ETRECIYYNA NWELERTNQS
51 GLERCEGEQD KRLHCYASWR NSSGTIELVK KGCWLDDFNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSRKEMT KNQVSLTCLV KGFYPSDIAV
301 EWESNGQPEN NYKTTPPVLK SDGSFFLYSK LTVDKSRWQQ GNVFSCSVMH
351 EALHNHYTQK SLSLSPGK (SEQ ID NO: 46)

The leader (signal) sequence and linker are underlined. To promote formation of the single-arm ActRIIB heterodimer Fc fusion rather than either of the possible homodimeric complexes (ActRIIB homodimer Fc fusion or homodimer Fc fusion), two amino acid substitutions (replacing acidic amino acids with lysine) can be introduced into the Fc domain of the ActRIIB fusion protein as indicated by double underline above. The amino acid sequence of SEQ ID NO: 46 may optionally be provided with the C-terminal lysine (K) removed.

This single-arm ActRIIB Fc fusion monomer is encoded by the following nucleic acid sequence (SEQ ID NO: 47):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGCTAGATGA CTTCAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGAA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGAAG TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 47)

A mature single-arm ActRIIB Fc fusion monomer (SEQ ID NO: 48) is as follows and may optionally be provided with the C-terminal lysine removed.

1 GRGEAETREC IYYNANWELE RTNQSGLERC EGEQDKRLHC YASWRNSSGT
51 IELVKKGCWL DDFNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 RKEMTKNQVS LTCLVKGFYP SDIAVEWESN GQPENNYKTT PPVLKSDGSF
301 FLYSKLTVDK SRWQQGNVFS CSVMHEALHN HYTQKSLSLS PGK
(SEQ ID NO: 48)

The complementary human G1Fc polypeptide (SEQ ID NO: 49) employs the TPA leader and is as follows:

1 MDAMKRGLCC VLLLCGAVFV SPGASNTKVD KRVTGGGTHT CPPCPAPELL
51 GGPSVFLFPP KPKDTLMISR TPEVTCVVVD VSHEDPEVKF NWYVDGVEVH
101 NAKTKPREEQ YNSTYRVVSV LTVLHQDWLN GKEYKCKVSN KALPAPIEKT
151 ISKAKGQPRE PQVYTLPPSR EEMTKNQVSL TCLVKGFYPS DIAVEWESNG
201 QPENNYDTTP PVLDSDGSFF LYSDLTVDKS RWQQGNVFSC SVMHEALHNH
251 YTQKSLSLSP GK (SEQ ID NO: 49)

The leader sequence is underlined, and an optional N-terminal extension of the Fc polypeptide is indicated by double underline. To promote formation of the single-arm ActRIIB heterodimer Fc fusion rather than either of the possible homodimeric fusions, two amino acid substitutions (replacing lysines with anionic residues) can be introduced into the monomeric Fc polypeptide as indicated by double underline above. The amino acid sequence of SEQ ID NO: 49 may optionally be provided with the C-terminal lysine removed.

This complementary Fc polypeptide is encoded by the following nucleic acid (SEQ ID NO: 50).

1 ATGGATGCAA TGAAGAGAGG GCTCTGCTGT GTGCTGCTGC TGTGTGGAGC
51 AGTCTTCGTT TCGCCCGGCG CCAGCAACAC CAAGGTGGAC AAGAGAGTTA
101 CCGGTGGTGG AACTCACACA TGCCCACCGT GCCCAGCACC TGAACTCCTG
151 GGGGGACCGT CAGTCTTCCT CTTCCCCCCA AAACCCAAGG ACACCCTCAT
201 GATCTCCCGG ACCCCTGAGG TCACATGCGT GGTGGTGGAC GTGAGCCACG
251 AAGACCCTGA GGTCAAGTTC AACTGGTACG TGGACGGCGT GGAGGTGCAT
301 AATGCCAAGA CAAAGCCGCG GGAGGAGCAG TACAACAGCA CGTACCGTGT
351 GGTCAGCGTC CTCACCGTCC TGCACCAGGA CTGGCTGAAT GGCAAGGAGT
401 ACAAGTGCAA GGTCTCCAAC AAAGCCCTCC CAGCCCCCAT CGAGAAAACC
451 ATCTCCAAAG CCAAAGGGCA GCCCCGAGAA CCACAGGTGT ACACCCTGCC
501 CCCATCCCGG GAGGAGATGA CCAAGAACCA GGTCAGCCTG ACCTGCCTGG
551 TCAAAGGCTT CTATCCCAGC GACATCGCCG TGGAGTGGGA GAGCAATGGG
601 CAGCCGGAGA ACAACTACGA CACCACGCCT CCCGTGCTGG ACTCCGACGG
651 CTCCTTCTTC CTCTATAGCG ACCTCACCGT GGACAAGAGC AGGTGGCAGC
701 AGGGGAACGT CTTCTCATGC TCCGTGATGC ATGAGGCTCT GCACAACCAC
751 TACACGCAGA AGAGCCTCTC CCTGTCTCCG GGTAAA
(SEQ ID NO: 50)

The sequence of a mature monomeric Fc polypeptide is as follows (SEQ ID NO: 51) and may optionally be provided with the C-terminal lysine removed.

1 SNTKVDKRVT GGGTHTCPPC PAPELLGGPS VFLFPPKPKD TLMISRTPEV
51 TCVVVDVSHE DPEVKFNWYV DGVEVHNAKT KPREEQYNST YRVVSVLTVL
101 HQDWLNGKEY KCKVSNKALP APIEKTISKA KGQPREPQVY TLPPSREEMT
151 KNQVSLTCLV KGFYPSDIAV EWESNGQPEN NYDTTPPVLD SDGSFFLYSD
201 LTVDKSRWQQ GNVFSCSVMH EALHNHYTQK SLSLSPGK
(SEQ ID NO: 51)

The single-arm ActRIIB Fc fusion monomer and monomeric Fc polypeptide of SEQ ID NO: 48 and SEQ ID NO: 51, respectively, may be co-expressed and purified from a CHO cell line to give rise to a single-arm ActRIIB heterodimer Fc fusion.

In another approach to promote the formation of heteromultimers using asymmetric Fc fusion polypeptides, the Fc domains are altered to introduce complementary hydrophobic interactions and an additional intermolecular disulfide bond, as illustrated in the single-arm ActRIIB Fc fusion monomer and monomeric Fc polypeptide sequences of SEQ ID NOs: 60-61, and 62-63, respectively.

The single-arm ActRIIB Fc fusion monomer sequence (SEQ ID NO: 60) employs the TPA leader and is shown below:

1 MDAMKRGLCC VLLLCGAVFV SPGASGRGEA ETRECIYYNA NWELERTNQS
51 GLERCEGEQD KRLHCYASWR NSSGTIELVK KGCWLDDFNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPCREEMT KNQVSLWCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 60)

The leader sequence and linker are underlined. To promote formation of the single-arm ActRIIB heterodimer Fc fusion rather than either of the possible homodimeric complexes, two amino acid substitutions (replacing a serine with a cysteine and a threonine with a tryptophan) can be introduced into the Fc domain of the fusion protein as indicated by double underline above. The amino acid sequence of SEQ ID NO: 60 may optionally be provided with the C-terminal lysine removed.

A mature single-arm ActRIIB Fc fusion monomer is as follows:

1 GRGEAETREC IYYNANWELE RTNQSGLERC EGEQDKRLHC YASWRNSSGT
51 IELVKKGCWL DDFNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPC
251 REEMTKNQVS LWCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 61)

The complementary form of monomeric Fc polypeptide (SEQ ID NO: 62) uses the TPA leader and is as follows.

1 MDAMKRGLCC VLLLCGAVFV SPGASNTKVD KRVTGGGTHT CPPCPAPELL
51 GGPSVFLFPP KPKDTLMISR TPEVTCVVVD VSHEDPEVKF NWYVDGVEVH
101 NAKTKPREEQ YNSTYRWSV LTVLHQDWLN GKEYKCKVSN KALPAPIEKT
151 ISKAKGQPRE PQVCTLPPSR EEMTKNQVSL SCAVKGFYPS DIAVEWESNG
201 QPENNYKTTP PVLDSDGSFF LVSKLTVDKS RWQQGNVFSC SVMHEALHNH
251 YTQKSLSLSP GK (SEQ ID NO: 62)

The leader sequence is underlined, and an optional N-terminal extension of the Fc polypeptide is indicated by double underline. To promote formation of the single-arm ActRIIB heterodimer Fc fusion rather than either of the possible homodimeric complexes, four amino acid substitutions can be introduced into the monomeric Fc polypeptide as indicated by double underline above. The amino acid sequence of SEQ ID NO: 62 may optionally be provided with the C-terminal lysine removed.

A mature monomeric Fc polypeptide sequence (SEQ ID NO: 63) is as follows and may optionally be provided with the C-terminal lysine removed.

1 SNTKVDKRVT GGGTHTCPPC PAPELLGGPS VFLFPPKPKD TLMISRTPEV
51 TCVVVDVSHE DPEVKFNWYV DGVEVHNAKT KPREEQYNST YRVVSVLTVL
101 HQDWLNGKEY KCKVSNKALP APIEKTISKA KGQPREPQVC TLPPSREEMT
151 KNQVSLSCAV KGFYPSDIAV EWESNGQPEN NYKTTPPVLD SDGSFFLVSK
201 LTVDKSRWQQ GNVFSCSVMH EALHNHYTQK SLSLSPGK
(SEQ ID NO: 63)

The single-arm ActRIIB Fc fusion monomer and monomeric Fc polypeptide of SEQ ID NO: 61 and SEQ ID NO: 63, respectively, may be co-expressed and purified from a CHO cell line to give rise to a single-arm ActRIIB heterodimer Fc fusion.

Purification of various single-arm ActRIIB heterodimer Fc fusions could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

A Biacore™-based binding assay was used to compare ligand binding selectivity of the single-arm ActRIIB heterodimer Fc fusion described above with that of ActRIIB homodimer Fc fusion. Single-arm ActRIIB homodimer Fc fusion and ActRIIB homodimer Fc fusion were independently captured onto the system using an anti-Fc antibody. Ligands were injected and allowed to flow over the captured receptor protein. Results are summarized in the table below, in which ligand off-rates (k_{d}) typically associated with the most effective ligand traps are denoted by bold text.

Ligand binding by single-arm ActRIIB homodimer Fc fusion compared to ActRIIB homodimer Fc fusion homodimer

| Ligand | ActRIIB homodimer Fc fusion | | | Single-arm ActRIIB homodimer Fc fusion | | |
|---|---|---|---|---|---|---|
| | kₐ (⅟Ms) | k_{d} (⅟s) | K_{D} (pM) | kₐ (⅟Ms) | k_{d} (⅟s) | K_{D} (pM) |
| Activin A | 1.2 × 10⁷ | 2.3 × 10⁻⁴ | 19 | 3.0 × 10⁷ | 3.0 × 10⁻³ | 99 |
| Activin B | 5.1 × 10⁶ | 1.0 × 10⁻⁴ | 20 | 3.5 × 10⁶ | 4.2 × 10⁻⁴ | 120 |
| BMP6 | 3.2 × 10⁷ | 6.8 × 10⁻³ | 210 | 4.2 × 10⁷ | 2.9 × 10⁻² | 690 |
| BMP9 | 1.4 × 10⁷ | 1.1 × 10⁻³ | 78 | No binding | | |
| BMP10 | 2.3 × 10⁷ | 2.6 × 10⁻⁴ | 11 | 8.0 × 10⁷ | 9.7 × 10⁻³ | 120 |
| GDF3 | 1.4 × 10⁶ | 2.2 × 10⁻³ | 1500 | 1.1 × 10⁶ | 1.3 × 10⁻² | 12000 |
| GDF8 | 8.3 × 10⁵ | 2.3 × 10⁻⁴ | 280 | 3.5 × 10⁶ | 1.0 × 10⁻³ | 290 |
| GDF11 | 5.0 × 10⁷ | 1.1 × 10⁻⁴ | 2 | 3.6 × 10⁷ | 7.2 × 10⁻⁴ | 20 |

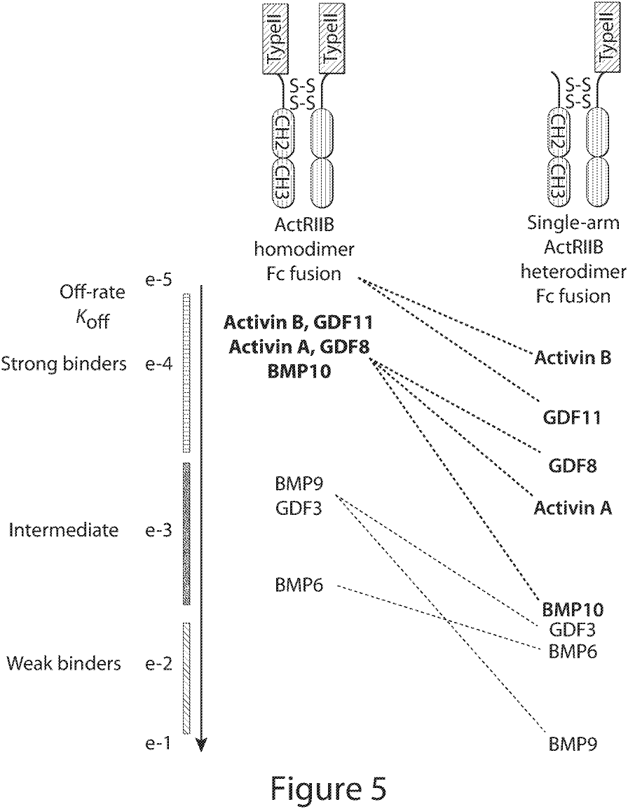

These comparative binding data demonstrate that single-arm ActRIIB heterodimer Fc fusion has greater ligand selectivity than ActRIIB homodimer Fc fusion. Whereas ActRIIB homodimer Fc fusion binds strongly to five important ligands (see cluster of activin A, activin B, BMP10, GDF8, and GDF11 in FIG. 5), single-arm ActRIIB heterodimer Fc fusion discriminates more readily among these ligands. Thus, single-arm ActRIIB heterodimer Fc fusion binds strongly to activin B and GDF11 and with intermediate strength to GDF8 and activin A. In further contrast to ActRIIB homodimer Fc fusion, single-arm ActRIIB heterodimer Fc fusion displays only weak binding to BMP10 and no binding to BMP9. See FIG. 5.

These results indicate that single-arm ActRIIB heterodimer Fc fusion is a more selective antagonist than ActRIIB homodimer Fc fusion. Accordingly, single-arm ActRIIB heterodimer Fc fusion will be more useful than ActRIIB homodimer Fc fusion in certain applications where such selective antagonism is advantageous. Examples include therapeutic applications where it is desirable to retain antagonism of one or more of activin A, activin B, GDF8, and GDF11 but minimize antagonism of one or more of BMP9, BMP10, BMP6, and GDF3. Selective inhibition of ligands in the former group would be particularly advantageous therapeutically because they constitute a subfamily which tends to differ functionally from the latter group and its associated set of clinical conditions.

### Example 2. Generation and Characterization of a Single-Arm ActRIIA Heterodimer Fc Fusion

Applicants constructed a soluble single-arm ActRIIA heterodimer Fc fusion comprising a monomeric Fc polypeptide with a short N-terminal extension and a second polypeptide in which the extracellular domain of human ActRIIA was fused to a separate Fc domain with a linker positioned between the extracellular domain and this second Fc domain. The individual constructs are referred to as monomeric Fc polypeptide and single-arm ActRIIA Fc fusion monomer, respectively, and the sequences for each are provided below.

Formation of a single-arm ActRIIA heterodimer Fc fusion may be guided by approaches similar to those described for single-arm ActRIIB heterodimer Fc fusion in Example 1. In a first approach, illustrated in the single-arm ActRIIA Fc fusion monomer and monomeric Fc polypeptide sequences of SEQ ID NOs: 55-57 and 49-51, respectively, one Fc domain is altered to introduce cationic amino acids at the interaction face, while the other Fc domain is altered to introduce anionic amino acids at the interaction face.

The single-arm ActRIIA Fc fusion monomer employs the TPA leader and is as follows:

1 MDAMKRGLCC VLLLCGAVFV SPGAAILGRS ETQECLFFNA NWEKDRTNQT
51 GVEPCYGDKD KRRHCFATWK NISGSIEIVK QGCWLDDINC YDRTDCVEKK
101 DSPEVYFCCC EGNMCNEKFS YFPEMEVTQP TSNPVTPKPP TGGGTHTCPP
151 CPAPELLGGP SVFLFPPKPK DTLMISRTPE VTCVVVDVSH EDPEVKFNWY
201 VDGVEVHNAK TKPREEQYNS TYRVVSVLTV LHQDWLNGKE YKCKVSNKAL
251 PAPIEKTISK AKGQPREPQV YTLPPSRKEM TKNQVSLTCL VKGFYPSDIA
301 VEWESNGQPE NNYKTTPPVL KSDGSFFLYS KLTVDKSRWQ QGNVFSCSVM
351 HEALHNHYTQ KSLSLSPGK (SEQ ID NO: 55)

The leader and linker sequences are underlined. To promote formation of the single-arm ActRIIA heterodimer Fc fusion rather than either of the possible homodimeric complexes (ActRIIA homodimer Fc fusion or Fc homodimeric fusions), two amino acid substitutions (replacing anionic residues with lysines) can be introduced into the Fc domain of the fusion polypeptide as indicated by double underline above. The amino acid sequence of SEQ ID NO: 55 may optionally be provided with the C-terminal lysine removed.

This single-arm ActRIIA Fc fusion monomer is encoded by the following nucleic acid (SEQ ID NO: 56).

1 ATGGATGCAA TGAAGAGAGG GCTCTGCTGT GTGCTGCTGC TGTGTGGAGC
51 AGTCTTCGTT TCGCCCGGCG CCGCTATACT TGGTAGATCA GAAACTCAGG
101 AGTGTCTTTT CTTTAATGCT AATTGGGAAA AAGACAGAAC CAATCAAACT
151 GGTGTTGAAC CGTGTTATGG TGACAAAGAT AAACGGCGGC ATTGTTTTGC
201 TACCTGGAAG AATATTTCTG GTTCCATTGA AATAGTGAAA CAAGGTTGTT
251 GGCTGGATGA TATCAACTGC TATGACAGGA CTGATTGTGT AGAAAAAAAA
301 GACAGCCCTG AAGTATATTT CTGTTGCTGT GAGGGCAATA TGTGTAATGA
351 AAAGTTTTCT TATTTTCCGG AGATGGAAGT CACACAGCCC ACTTCAAATC
401 CAGTTACACC TAAGCCACCC ACCGGTGGTG GAACTCACAC ATGCCCACCG
451 TGCCCAGCAC CTGAACTCCT GGGGGGACCG TCAGTCTTCC TCTTCCCCCC
501 AAAACCCAAG GACACCCTCA TGATCTCCCG GACCCCTGAG GTCACATGCG
551 TGGTGGTGGA CGTGAGCCAC GAAGACCCTG AGGTCAAGTT CAACTGGTAC
601 GTGGACGGCG TGGAGGTGCA TAATGCCAAG ACAAAGCCGC GGGAGGAGCA
651 GTACAACAGC ACGTACCGTG TGGTCAGCGT CCTCACCGTC CTGCACCAGG
701 ACTGGCTGAA TGGCAAGGAG TACAAGTGCA AGGTCTCCAA CAAAGCCCTC
751 CCAGCCCCCA TCGAGAAAAC CATCTCCAAA GCCAAAGGGC AGCCCCGAGA
801 ACCACAGGTG TACACCCTGC CCCCATCCCG GAAGGAGATG ACCAAGAACC
851 AGGTCAGCCT GACCTGCCTG GTCAAAGGCT TCTATCCCAG CGACATCGCC
901 GTGGAGTGGG AGAGCAATGG GCAGCCGGAG AACAACTACA AGACCACGCC
951 TCCCGTGCTG AAGTCCGACG GCTCCTTCTT CCTCTATAGC AAGCTCACCG
1001 TGGACAAGAG CAGGTGGCAG CAGGGGAACG TCTTCTCATG CTCCGTGATG
1051 CATGAGGCTC TGCACAACCA CTACACGCAG AAGAGCCTCT CCCTGTCTCC
1101 GGGTAAA (SEQ ID NO: 56)

A mature single-arm ActRIIA Fc fusion monomer sequence is as follows (SEQ ID NO: 57) and may optionally be provided with the C-terminal lysine removed.

1 ILGRSETQEC LFFNANWEKD RTNQTGVEPC YGDKDKRRHC FATWKNISGS
51 IEIVKQGCWL DDINCYDRTD CVEKKDSPEV YFCCCEGNMC NEKFSYFPEM
101 EVTQPTSNPV TPKPPTGGGT HTCPPCPAPE LLGGPSVFLF PPKPKDTLMI
151 SRTPEVTCVV VDVSHEDPEV KFNWYVDGVE VHNAKTKPRE EQYNSTYRVV
201 SVLTVLHQDW LNGKEYKCKV SNKALPAPIE KTISKAKGQP REPQVYTLPP
251 SRKEMTKNQV SLTCLVKGFY PSDIAVEWES NGQPENNYKT TPPVLKSDGS
301 FFLYSKLTVD KSRWQQGNVF SCSVMHEALH NHYTQKSLSL SPGK
(SEQ ID NO: 57)

As described in Example 1, the complementary form of monomeric human G1Fc polypeptide (SEQ ID NO: 49) employs the TPA leader and incorporates an optional N-terminal extension. To promote formation of the single-arm ActRIIA heterodimer Fc fusion rather than either of the possible homodimeric complexes, two amino acid substitutions (replacing lysines with anionic residues) can be introduced into the monomeric Fc polypeptide. The amino acid sequence of SEQ ID NO: 49 may optionally be provided without the C-terminal lysine. This complementary Fc polypeptide is encoded by the nucleic acid of SEQ ID NO: 50, and a mature monomeric Fc polypeptide (SEQ ID NO: 51) may optionally be provided with the C-terminal lysine removed.

The single-arm ActRIIA Fc fusion monomer and monomeric Fc polypeptide of SEQ ID NO: 57 and SEQ ID NO: 51, respectively, may be co-expressed and purified from a CHO cell line to give rise to a single-arm ActRIIA heterodimer Fc fusion.

In another approach to promoting the formation of heteromultimers using asymmetric Fc fusion polypeptides, the Fc domains are altered to introduce complementary hydrophobic interactions and an additional intermolecular disulfide bond as illustrated in the single-arm ActRIIA Fc fusion monomer and Fc polypeptide sequences of SEQ ID NOs: 58-59 and 62-63, respectively.

The single-arm ActRIIA Fc fusion monomer (SEQ ID NO: 58) uses the TPA leader and is as follows:

1 MDAMKRGLCC VLLLCGAVFV SPGAAILGRS ETQECLFFNA NWEKDRTNQT
1 GVEPCYGDKD KRRHCFATWK NISGSIEIVK QGCWLDDINC YDRTDCVEKK
101 DSPEVYFCCC EGNMCNEKFS YFPEMEVTQP TSNPVTPKPP TGGGTHTCPP
151 CPAPELLGGP SVFLFPPKPK DTLMISRTPE VTCVVVDVSH EDPEVKFNWY
201 VDGVEVHNAK TKPREEQYNS TYRVVSVLTV LHQDWLNGKE YKCKVSNKAL
251 PAPIEKTISK AKGQPREPQV YTLPPCREEM TKNQVSLWCL VKGFYPSDIA
301 VEWESNGQPE NNYKTTPPVL DSDGSFFLYS KLTVDKSRWQ QGNVFSCSVM
351 HEALHNHYTQ KSLSLSPGK (SEQ ID NO: 58)

The leader sequence and linker are underlined. To promote formation of the single-arm ActRIIA heterodimer Fc fusion rather than either of the possible homodimeric complexes, two amino acid substitutions (replacing a serine with a cysteine and a threonine with a tryptophan) can be introduced into the Fc domain of the single-arm ActRIIA Fc fusion monomer as indicated by double underline above. The amino acid sequence of SEQ ID NO: 58 may optionally be provided with the C-terminal lysine removed.

A mature single-arm ActRIIA Fc fusion monomer (SEQ ID NO: 59) is as follows and may optionally be provided with the C-terminal lysine removed.

1 ILGRSETQEC LFFNANWEKD RTNQTGVEPC YGDKDKRRHC FATWKNISGS
51 IEIVKQGCWL DDINCYDRTD CVEKKDSPEV YFCCCEGNMC NEKFSYFPEM
101 EVTQPTSNPV TPKPPTGGGT HTCPPCPAPE LLGGPSVFLF PPKPKDTLMI
151 SRTPEVTCW VDVSHEDPEV KFNWYVDGVE VHNAKTKPRE EQYNSTYRW
201 SVLTVLHQDW LNGKEYKCKV SNKALPAPIE KTISKAKGQP REPQVYTLPP
251 CREEMTKNQV SLWCLVKGFY PSDIAVEWES NGQPENNYKT TPPVLDSDGS
301 FFLYSKLTVD KSRWQQGNVF SCSVMHEALH NHYTQKSLSL SPGK
(SEQ ID NO: 59)

As described in Example 1, the complementary form of monomeric human G1Fc polypeptide (SEQ ID NO: 62) employs the TPA leader and incorporates an optional N-terminal extension. To promote formation of the single-arm ActRIIA heterodimer Fc fusion rather than either of the possible homodimeric complexes, four amino acid substitutions can be introduced into the monomeric Fc polypeptide as indicated. The amino acid sequence of SEQ ID NO: 62 and a mature G1Fc polypeptide (SEQ ID NO: 63) may optionally be provided with the C-terminal lysine removed.

The single-arm ActRIIA Fc fusion monomer and monomeric Fc polypeptide of SEQ ID NO: 59 and SEQ ID NO: 63, respectively, may be co-expressed and purified from a CHO cell line to give rise to a single-arm ActRIIA homodimer Fc fusion.

Purification of various single-arm ActRIIA heterodimer Fc fusions could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

A Biacore™-based binding assay was used to compare ligand binding selectivity of the single-arm ActRIIA heterodimer Fc fusions described above with that of an ActRIIA homodimer Fc fusion. The single-arm ActRIIA heterodimer Fc fusions and ActRIIA homodimer Fc fusions were independently captured onto the system using an anti-Fc antibody. Ligands were injected and allowed to flow over the captured receptor protein. Results are summarized in the table below, in which ligand off-rates (k_{d}) typically associated with the most effective ligand traps are denoted by bold text.

Ligand binding of single-arm ActRIIA-Fc heterodimer Fc fusion compared to ActRIIA homodimer Fc fusion

| Ligand | ActRIIA homodimer Fc fusion | | | Single-arm ActRIIA-Fc heterodimer Fc fusion | | |
|---|---|---|---|---|---|---|
| | kₐ (⅟Ms) | k_{d} (⅟s) | K_{D} (pM) | kₐ (⅟Ms) | k_{d} (⅟s) | K_{D} (pM) |
| Activin A | 1.4 × 10⁷ | 6.2 × 10⁻⁴ | 45 | 3.0 × 10⁷ | 9.0 × 10⁻⁴ | 30 |
| Activin B | 7.9 × 10⁶ | 2.0 × 10⁻⁴ | 25 | 2.9 × 10⁷ | 1.4 × 10⁻³ | 46 |
| BMP5 | 4.0 × 10⁶ | 4.5 × 10⁻³ | 1100 | 4.8 × 10⁷ | 5.8 × 10⁻² | 1200 |
| BMP10 | 2.9 × 10⁷ | 2.5 × 10⁻³ | 86 | 2.3 × 10⁷ | 5.9 × 10⁻³ | 250 |
| GDF8 | 1.4 × 10⁷ | 1.4 × 10⁻³ | 99 | 4.7 × 10⁶ | 5.0 × 10⁻³ | 1100 |
| GDF11 | 2.6 × 10⁷ | 7.2 × 10⁻⁴ | 28 | 4.9 × 10⁷ | 1.1 × 10⁻² | 220 |

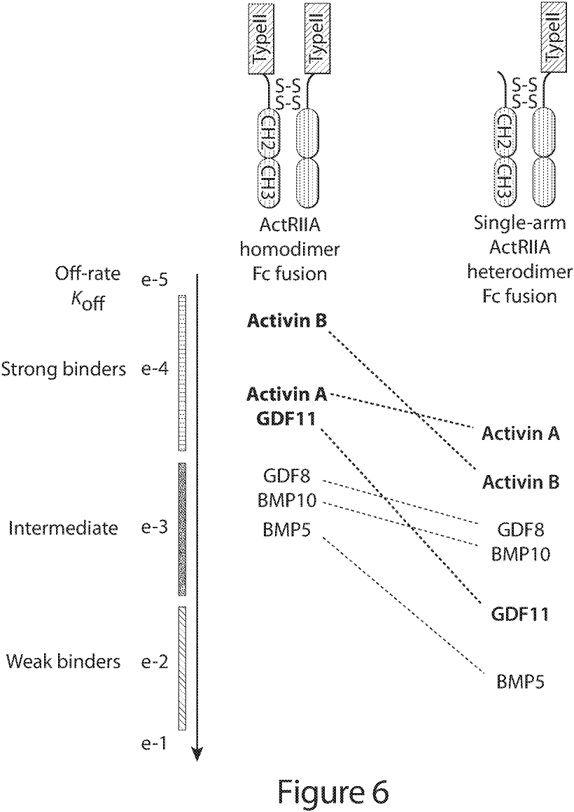

These comparative binding data indicate that a single-arm ActRIIA-Fc heterodimer Fc fusion has different ligand selectivity than an ActRIIA homodimer Fc fusion (and also different than single-armor homomeric ActRIIB-Fc - see Example 1). Whereas an ActRIIA homodimer Fc fusion exhibits preferential binding to activin B combined with strong binding to activin A and GDF11, single-arm ActRIIA heterodimer Fc fusion has a reversed preference for activin A over activin B combined with greatly enhanced selectivity for activin A over GDF11 (weak binder). See FIG. 6. In addition, single-arm ActRIIA heterodimer Fc fusion largely retains the intermediate binding to GDF8 and BMP10 observed with ActRIIA homodimer Fc fusion.

These results indicate that single-arm ActRIIA heterodimer Fc fusion is an antagonist with substantially altered ligand selectivity compared to ActRIIA homodimer Fc fusion. Accordingly, a single-arm ActRIIA heterodimer Fc fusion will be more useful than an ActRIIA homodimer Fc fusion in certain applications where such antagonism is advantageous. Examples include therapeutic applications where it is desirable to antagonize activin A preferentially over activin B while minimizing antagonism of GDF11.

Together the foregoing examples demonstrate that ActRIIA or ActRIIB polypeptides, when placed in the context of a single-arm heteromeric protein complex, form novel binding pockets that exhibit altered selectivity relative to either type of homomeric protein complex, allowing the formation of novel protein agents for possible use as therapeutic agents.

### Example 3. Effects of a Single-Arm ActRIIB Heterodimer Fc Fusion on Pulmonary Hypertension (PH) in the Sugen Hypoxia Rat Model

The effects of Single-arm ActRIIB heterodimer Fc fusion and sildenafil (a phosphodiesterase-5 inhibitor approved for the treatment of PAH) were examined the Sugen Hypoxia model of PAH. In this model, rats receive daily doses of semaxanib and are placed in a low oxygen environment (approximately 13% oxygen) to induce PAH 24 hours prior to start of therapy.

Rats were separated into different treatment groups (5-10 rats per group): 1) control rats (Tris buffered saline administered s.c. as 1 ml/kg, every three days), “Normal”; 2) treatment with semaxanib (200 mg/kg administered s.c. as a single dose daily)/hypoxia and Tris buffered saline (administered s.c. as 1 ml/kg, every three days) (vehicle treatment group), “PBS”; 3) treatment with a single-arm ActRIIB heterodimer Fc fusion (3 mg/kg administered s.c. every three days) and semaxanib (200 mg/kg administered s.c. as a single dose daily)/hypoxia, “sa IIB hd 3mpk”; 4) treatment with a single-arm ActRIIB heterodimer Fc fusion (5 mg/kg administered s.c. every three days) and semaxanib (200 mg/kg administered s.c. as a single dose daily)/hypoxia, “sa IIB hd 5mpk”; 5) treatment with a single-arm ActRIIB heterodimer Fc fusion (10 mg/kg administered s.c. every three days) and semaxanib (200 mg/kg administered s.c. as a single dose daily)/hypoxia, “sa IIB hd 10mpk”; and 6) treatment with sildenafil (30 mg/kg administered orally twice daily) and semaxanib (200 mg/kg administered s.c. as a single dose daily)/hypoxia, “Sildenafil”. Rats were treated for 28 days. Body weights were recorded prior to first dose on Day 1 and then weekly throughout the study.

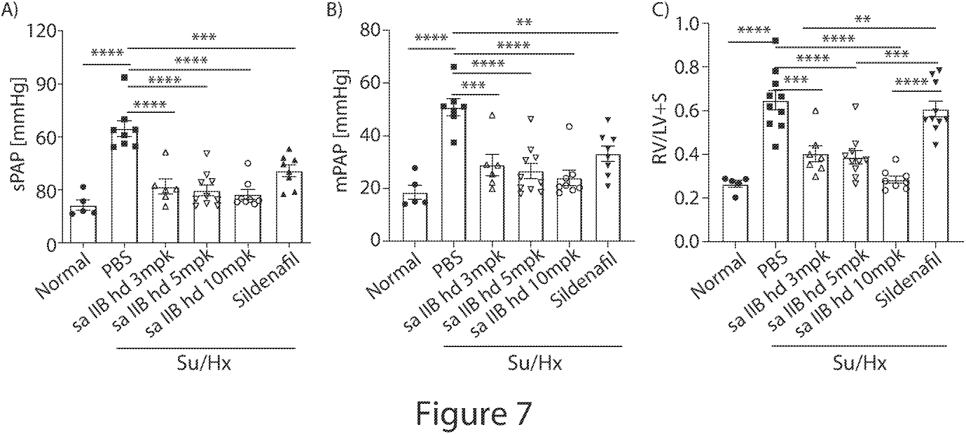

On day 28, rats were anesthetized by an intraperitoneal injection of ketamine/xylazine (80/10 mg/kg). An incision was made in the neck, and a jugular vein was isolated and ligated anteriorly. A fluid-filled pressure catheter was introduced into the right jugular vein to measure pulmonary artery pressure (PAP). FIG. 7A depicts the measured systolic pulmonary artery pressure (sPAP), while FIG. 7B depicts the calculated mean pulmonary artery pressure (mPAP). Another incision was made in the inguinal region, and femoral artery was isolated and ligated anteriorly. A Millar pressure catheter was introduced into a femoral artery to measure systolic arterial pressure, diastolic pressure, and heart rate. Mean arterial pressure (mPAP) (FIG. 7B) and right PAP were monitored using the Notocord HEM (Croissy sur Seine, France) v3.5 data capture system for approximately 5-10 minutes until stable measurements were obtained. During the measurements, rats were maintained at approximately 37° C. on a heating pad and body temperature was monitored throughout the procedure with a rectal temperature probe. At the conclusion of the procedure, rats were euthanized, and the hearts and lungs were removed. The entire heart was weighed. Next, the atria were removed and the left ventricle with septum (LV + S) was separated from the right ventricle (RV). The ventricles were weighed separately. Hypertrophy was assessed, in part, by calculating RV/LV + S (FIG. 7C). The lungs were also weighed.

Compared to control animals, semaxanib/hypoxia treated rats (“PBS”) were observed to have decreased body weight, elevated PAP, right heart hypertrophy, and increased lung weight, indicating establishment of PAH. Sildenafil treatment reduced mean pulmonary arterial pressure by 34.8% and decreased right heart hypertrophy by 6.3% compared to “PBS” animals. a single-arm ActRIIB heterodimer Fc fusion treatment was found have greater effects in treating PAH in this model compared to sildenafil. 3mpk, 5mpk, and 10mpk of a single-arm ActRIIB heterodimer Fc fusion treatments resulted in a reduction of mean pulmonary arterial pressure by 43.2%, 47.7%, and 52.8% and decreased right heart hypertrophy by 37.8%, 40.2%, and 56.1% respectively, compared to “PBS” animals.

Together, these data demonstrate that a single-arm ActRIIB heterodimer Fc fusion is effective to ameliorate various complications of PAH in the Sugen Hypoxia model. In particular, a single-arm ActRIIB heterodimer Fc fusion had a greater effect in reducing pulmonary artery pressure and right heart hypertrophy than was observed for sildenafil, which is an approved drug for the treatment of PAH.

### INCORPORATION BY REFERENCE

All publications and patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

We claim:

1. A method of treating pulmonary hypertension (PH) comprising administering a single-arm ActRIIB heteromultimer to a subject in need thereof, the heteromultimer comprising a first polypeptide covalently or non-covalently associated with a second polypeptide, wherein:
a. the first polypeptide comprises the amino acid sequence of a first member of an interaction pair and the amino acid sequence of ActRIIB; and
b. the second polypeptide comprises the amino acid sequence of a second member of the interaction pair, and wherein the second polypeptide does not comprise ActRIIB.

2. A method of treating pulmonary hypertension (PH) comprising administering a single-arm ActRIIA heteromultimer to a subject in need thereof, the heteromultimer comprising a first polypeptide covalently or non-covalently associated with a second polypeptide, wherein:
a. the first polypeptide comprises the amino acid sequence of a first member of an interaction pair and the amino acid sequence of ActRIIA; and
b. the second polypeptide comprises the amino acid sequence of a second member of the interaction pair, and wherein the second polypeptide does not comprise ActRIIA.

3. The method of claim 1, wherein the ActRIIB polypeptide comprises an amino acid sequence that is:
a. at least 70%, 80%, 85%, 90%, 91%, 92%, 93%, 94%, 94%, 95%, 96%, 97%, 98%, 99% or 100% identical to the sequence of any of SEQ ID Nos: 1, 2, 3, 4, 5, and 6; or
b. at least 70%, 80%, 85%, 90%, 91%, 92%, 93%, 94%, 94%, 95%, 96%, 97%, 98%, 99% or 100% identical to a polypeptide that begins at any one of amino acids 20, 21, 22, 23, 24, 25, 26, 27, 28, or 29 of SEQ ID NO: 1, and ends at any one of amino acids 109, 110, 111, 112, 113, 114, 115, 116, 117, 118, 119, 120, 121, 122, 123, 124, 125, 126, 127, 128, 129, 130, 131, 132, 133, or 134 of SEQ ID NO: 1.

4. The method of claim 3, wherein the ActRIIB polypeptide does not comprise an acidic amino acid at the position corresponding to L79 of SEQ ID NO: 1.

5. The method of claim 3, wherein the ActRIIB polypeptide does not comprise an aspartic acid (D) at the position corresponding to L79 of SEQ ID NO: 1.

6. The method of claim 2, wherein the ActRIIA polypeptide comprises an amino acid sequence that is:
a. at least 70%, 80%, 85%, 90%, 91%, 92%, 93%, 94%, 94%, 95%, 96%, 97%, 98%, 99% or 100% identical to the sequence of any of SEQ ID Nos: 9, 10, and 11; or
b. at least 70%, 80%, 85%, 90%, 91%, 92%, 93%, 94%, 94%, 95%, 96%, 97%, 98%, 99% or 100% identical to a polypeptide that begins at any one of amino acids 21, 22, 23, 24, 25, 26, 27, 28, 29, or 30 of SEQ ID NO: 9, and ends at any one of amino acids 110, 111, 112, 113, 114, 115, 116, 117, 118, 119, 120, 121, 122, 123, 124, 125, 126, 127, 128, 129, 130, 131, 132, 133, 134 or 135 of SEQ ID NO: 9.

7. The method of any one of claims 1-6, wherein the heteromultimer is a heterodimer.

8. The method of any of claims 1-7, wherein the first member of an interaction pair comprises a first constant region from an IgG heavy chain.

9. The method of any of claims 1-7, wherein the second member of an interaction pair comprises a second constant region from an IgG heavy chain.

10. The method of claim 8, wherein the first constant region from an IgG heavy chain is a first immunoglobulin Fc domain.

11. The method of claim 9, wherein the second constant region from an IgG heavy chain is a first immunoglobulin Fc domain.

12. The method of claim 8, wherein the first constant region from an IgG heavy chain comprises an amino acid sequence that is at least 70%, 80%, 85%, 90%, 95%, 97%, 98%, 99% or 100% identical to a sequence selected from any one of SEQ ID NOs: 14-28.

13. The method of claim 9, wherein the second constant region from an IgG heavy chain comprises an amino acid sequence that is at least 70%, 80%, 85%, 90%, 95%, 97%, 98%, 99% or 100% identical to a sequence selected from any one of SEQ ID NOs: 14-28.

14. The method of any of claims 1-13, wherein the first polypeptide comprises an amino acid sequence that is at least 70%, 80%, 85%, 90%, 95%, 97%, 98%, 99% or 100% identical to a sequence selected from any one of SEQ ID NOs: 46, 48, 55, 57, 58, 59, 60, and 61.

15. The method of any of claims 1-14, wherein the second polypeptide comprises an amino acid sequence that is at least 70%, 80%, 85%, 90%, 95%, 97%, 98%, 99% or 100% identical to a sequence selected from any one of SEQ ID NOs: 49, 51, 62, and 63.

16. The method of any one of claims 1, 3-5, or 7-15, wherein the single-arm ActRIIB heteromultimer comprises a linker domain positioned between the ActRIIB polypeptide and the first member of an interaction pair.

17. The method of any one of claims 2, 6, or 7-15, wherein the single-arm ActRIIA heteromultimer comprises a linker domain positioned between the ActRIIA polypeptide and the first member of an interaction pair.

18. The method of any one of claims 16 or 17, wherein the linker domain comprises an amino acid sequence selected from any one of SEQ ID NOs: 29-44.

19. The method of any one of claims 1-18, wherein the first polypeptide and/or second polypeptide comprises one or more modified amino acid residues selected from: a glycosylated amino acid, a PEGylated amino acid, a famesylated amino acid, an acetylated amino acid, a biotinylated amino acid, and an amino acid conjugated to a lipid moiety.

20. The method of any one of claims 1-19, wherein the first polypeptide and/or second polypeptide is glycosylated and has a glycosylation pattern obtainable from expression of the first polypeptide and/or second polypeptide in a CHO cell.

21. The method of any one of claims 1-20, wherein the heteromultimer binds to one or more ligands selected from the group consisting of activin A, activin B, GDF11, GDF8, GDF3, BMP5, BMP6, and BMP10.

22. The method of any one of claims 1, 3-5, or 7-16, 18-21, wherein the single-arm ActRIIB heteromultimer binds to activin B and GDF11.

23. The method of any one of claims 1, 3-5, or 7-16, 18-21, wherein the single-arm ActRIIB heteromultimer binds to GDF8 and activin A.

24. The method of any one of claims 2, 6, 7-15 or 17-21, wherein the single-arm ActRIIA heteromultimer binds to activin A.

25. The method of any one of claims 2, 6, 7-15, or 17-21, wherein the single-arm ActRIIA heteromultimer binds to GDF8.

26. The method of any one of claims 1-26, wherein the heteromultimer inhibits the activity of one or more ligands in a cell-based assay.

27. The method of any one of claims 1-26, wherein the pulmonary hypertension is pulmonary arterial hypertension.

28. The method of any one of claims 1-27, comprising further administering to the subject an additional active agent and/or supportive therapy for treating pulmonary hypertension.

29. The method of claim 28, wherein the additional active agent and/or supportive therapy for treating pulmonary hypertension is selected from the group consisting of: prostacyclin and derivatives thereof (e.g., epoprostenol, treprostinil, and iloprost); prostacyclin receptor agonists (e.g., selexipag); endothelin receptor antagonists (e.g., thelin, ambrisentan, macitentan, and bosentan); calcium channel blockers (e.g., amlodipine, diltiazem, and nifedipine; anticoagulants (e.g., warfarin); diuretics; oxygen therapy; atrial septostomy; pulmonary thromboendarterectomy; phosphodiesterase type 5 inhibitors (e.g., sildenafil and tadalafil); activators of soluble guanylate cyclase (e.g., cinaciguat and riociguat); ASK-1 inhibitors (e.g., CIIA; SCH79797; GS-4997; MSC2032964A; 3H-naphtho[1,2,3-de]quiniline-2,7-diones, NQDI-1; 2-thioxo-thiazolidines, 5-bromo-3-(4-oxo-2-thioxo-thiazolidine-5-ylidene)-1,3-dihydro-indol-2-one); NF-κB antagonists (e.g., dh404, CDDO-epoxide; 2.2-difluoropropionamide; C28 imidazole (CDDO-Im); 2-cyano-3,12-dioxoolean-1,9-dien-28-oic acid (CDDO); 3-Acetyloleanolic Acid; 3-Triflouroacetyloleanolic Acid; 28-Methyl-3-acetyloleanane; 28-Methyl-3-trifluoroacetyloleanane; 28-Methyloxyoleanolic Acid; SZC014; SCZ015; SZC017; PEGylated derivatives of oleanolic acid; 3-O-(beta-D-glucopyranosyl) oleanolic acid; 3-O-[beta-D-glucopyranosyl-(1-->3)-beta-D-glucopyranosyl] oleanolic acid; 3-O-[beta-D-glucopyranosyl-(1-->2)-beta-D-glucopyranosyl] oleanolic acid; 3-O-[beta-D-glucopyranosyl-(1-->3)-beta-D-glucopyranosyl] oleanolic acid 28-O-beta-D-glucopyranosyl ester; 3-O-[beta-D-glucopyranosyl-(1-->2)-beta-D-glucopyranosyl] oleanolic acid 28-O-beta-D-glucopyranosyl ester; 3-O-[a-L-rhamnopyranosyl-(1-->3)-beta-D-glucuronopyranosyl] oleanolic acid; 3-O-[alpha-L-rhamnopyranosyl-(1-->3)-beta-D-glucuronopyranosyl] oleanolic acid 28-O-beta-D-glucopyranosyl ester; 28-O-β-D-glucopyranosyl-oleanolic acid; 3-O-β-D-glucopyranosyl (1→3)-β-D-glucopyranosiduronic acid (CS1); oleanolic acid 3-O-β-D-glucopyranosyl (1→3)-β-D-glucopyranosiduronic acid (CS2); methyl 3,11-dioxoolean-12-en-28-olate (DIOXOL); ZCVI₄-2; Benzyl 3-dehydr-oxy-1,2,5-oxadiazolo[3′,4′:2,3]oleanolate); lung and/or heart transplantation.

30. The method of any one of claims 1-29, wherein the subject has resting pulmonary arterial pressure (PAP) of at least 25 mm Hg (e.g., 25, 30, 35, 40, 45, or 50 mm Hg).

31. The method of any one of claims 1-30, wherein the method reduces PAP in the subj ect.

32. The method of claim 31, wherein the method reduces PAP by at least 3 mmHg (e.g., at least 3, 5, 7, 10, 12, 15, 20, or 25 mm Hg) in the subject.

33. The method of any one of claims 1-32, wherein the method reduces pulmonary vascular resistance in the subject.

34. The method of any one of claims 1-33, wherein the method increases pulmonary capillary wedge pressure.

35. The method of any one of claims 1-34, wherein the method increases left ventricular end-diastolic pressure.

36. The method of any one of claims 1-35, wherein the method increases exercise capacity of the subject.

37. The method of claim 36, wherein the method increases the subject’s 6-minute walk distance.

38. The method of claim 37, wherein the method increases the subject’s 6-minute walk distance by at least 10 meters (e.g., at least 10, 20, 30, 40, 50, 60, 70, 80, 90, 100 or more meters).

39. The method any one of claims 1-38, wherein the method reduces the subject’s Borg dyspnea index (BDI).

40. The method of claim 39, wherein the method reduces the subject’s BDI by at least 0.5 index points (e.g., at least 0.5, 1, 1.5, 2, 2.5, 3, 3.5, 4, 4.5, 5, 5.5, 6, 6.5, 7, 7.5, 8, 8.5, 9, 9.5, or 10 index points).

41. The method of any one of claims 1-40, wherein the subject has Functional Class I, Class II, Class III, or Class IV pulmonary hypertension as recognized by the World Health Organization.

42. The method of claim 41, wherein the method prevents or delays pulmonary hypertension Functional Class progression (e.g., prevents or delays progression from Functional Class I to Class II, Class II to Class III, or Class III to Class IV pulmonary hypertension as recognized by the World Health Organization).

43. The method of claim 41, wherein the method promotes or increases pulmonary hypertension Functional Class regression (e.g., promotes or increases regression from Class IV to Class III, Class III to Class II, or Class II to Class I pulmonary hypertension as recognized by the World Health Organization).

44. The method of any one of claims 1-43, wherein the method decreases pulmonary arterial pressure in the subject.

45. The method of claim 44, wherein the method decreases pulmonary arterial pressure in the subject by at least 10% (e.g., 10%, 15%, 20%, 25%, 30%, 35%, 40%, 45%, 50%, 55%, 60%, 65%, 70%, 75%, or at least 80%).

46. The method of any one of claims 1-45, wherein the method decreases ventricle hypertrophy in the subject.

47. The method of claim 46, wherein the method decreases ventricle hypertrophy in the subject by at least 10% (e.g., 10%, 15%, 20%, 25%, 30%, 35%, 40%, 45%, 50%, 55%, 60%, 65%, 70%, 75%, or at least 80%).

48. The method of any one of claims 1-47, wherein the method decreases smooth muscle hypertrophy in the subject.

49. The method of claim 48, wherein the method decreases smooth muscle hypertrophy in the subject by at least 10% (e.g., 10%, 15%, 20%, 25%, 30%, 35%, 40%, 45%, 50%, 55%, 60%, 65%, 70%, 75%, or at least 80%).

50. The method of any one of claims 1-49, wherein the method decreases pulmonary arteriole muscularity in the subject.

51. The method of claim 50, wherein the method decreases pulmonary arteriole muscularity in the subject by at least 10% (e.g., 10%, 15%, 20%, 25%, 30%, 35%, 40%, 45%, 50%, 55%, 60%, 65%, 70%, 75%, or at least 80%).

52. The method of any one of claims 1-51, wherein the method decreases pulmonary vascular resistance in the subject.

53. The method of claim 52, wherein the method decreases pulmonary vascular resistance in the subject by at least 10% (e.g., 10%, 15%, 20%, 25%, 30%, 35%, 40%, 45%, 50%, 55%, 60%, 65%, 70%, 75%, or at least 80%).

54. The method of any one of claims 1-53, wherein the subject has pulmonary arterial hypertension and has Functional Class II or Class III pulmonary hypertension in accordance with the World Health Organization’s functional classification system for pulmonary hypertension.

55. The method of any one of claims 1-54, wherein the subject has pulmonary arterial hypertension that is classified as one or more subtypes selected from the group consisting of: idiopathic or heritable pulmonary arterial hypertension, drug- and/or toxin-induced pulmonary hypertension, pulmonary hypertension associated with connective tissue disease, and pulmonary hypertension associated with congenital systemic-to-pulmonary shunts at least 1 year following shunt repair.

56. The method of any one of claims 1-55, wherein the subject has been treated with one or more vasodilators.

57. The method of any one of claims 1-56, wherein the subject has been treated with one or more agents selected from the group consisting of: phosphodiesterase type 5 inhibitors, soluble guanylate cyclase stimulators, prostacyclin receptor agonist, and endothelin receptor antagonists.

58. The method of claim 57, wherein the one or more agents is selected from the group consisting of: bosentan, sildenafil, beraprost, macitentan, selexipag, epoprostenol, treprostinil, iloprost, ambrisentan, and tadalafil.

59. The method of any one of claims 1-58, wherein the method further comprises administration of one or more vasodilators.

60. The method of any one of claims 1-59, wherein the method further comprises administration of one or more agents selected from the group consisting of: phosphodiesterase type 5 inhibitors, soluble guanylate cyclase stimulators, prostacyclin receptor agonist, and endothelin receptor antagonists.

61. The method of claim 59, wherein the one or more agents is selected from the group consisting of: bosentan, sildenafil, beraprost, macitentan, selexipag, epoprostenol, treprostinil, iloprost, ambrisentan, and tadalafil.

62. The method of any one of claims 1-61, wherein the subject has a 6-minute walk distance from 150 to 400 meters.

63. The method of any one of claims 1-62, wherein the subject has a hemoglobin level from >8 and <15 g/dl.

64. The method of any one of claims 1-63, wherein the method delays clinical worsening of pulmonary hypertension.

65. The method of claim 64, wherein the method delays clinical worsening of pulmonary hypertension in accordance with the World Health Organization’s functional classification system for pulmonary hypertension.

66. The method of any one of claims 1-65, wherein the method reduces the risk of hospitalization for one or more complications associated with pulmonary hypertension.
